(19) United States
(12) Patent Application Publication
(10) Pub. No.: US 2023/0377685 A1
(43) Pub. Date: Nov. 23, 2023

(54) METHODS OF CLASSIFYING THE DIFFERENTIATION STATE OF CELLS AND RELATED COMPOSITIONS OF DIFFERENTIATED CELLS

(71) Applicant: Aspen Neuroscience, Inc., San Diego, CA (US)

(72) Inventors: Jim MOSSMAN, San Diego, CA (US); Derren BARKEN, San Diego, CA (US); Roy WILLIAMS, San Diego, CA (US)

(73) Assignee: Aspen Neuroscience, Inc., San Diego, CA (US)

(21) Appl. No.: 18/135,069

(22) Filed: Apr. 14, 2023

Related U.S. Application Data
(60) Provisional application No. 63/353,525, filed on Jun. 17, 2022.
(60) Provisional application No. 63/331,783, filed on Apr. 15, 2022.

(51) Int. Cl. G16B 25/10; A61K 35/12; C12N 5/0793; G01N 33/68; G16B 40/20

(52) U.S. Cl. CPC G16B 25/10; A61K 35/12; C12N 5/0619; G01N 33/6869; G16B 40/20

(57) ABSTRACT

Provided herein are methods for classifying the differentiation state of an in vitro population of cells, for instance an in vitro population of neuronal cells, as well as methods for selecting and/or implanting an in vitro population of cells having a desired differentiation state. Also provided herein are computing devices for performing the provided methods as well as related compositions, articles of manufacture, and kits, including for use in methods of treating a subject having a disease or condition, such as a neurodegenerative disease, for instance Parkinson's disease.

### CROSS-REFERENCE TO RELATED APPLICATIONS

This application claims priority to U.S. Provisional Application No. 63/331,783, filed Apr. 15, 2022, entitled “METHODS OF CLASSIFYING THE DIFFERENTIATION STATE OF CELLS AND RELATED COMPOSITIONS OF DIFFERENTIATED CELLS,” and U.S. Provisional Application No. 63/353,525, filed Jun. 17, 2022, entitled “METHODS OF CLASSIFYING THE DIFFERENTIATION STATE OF CELLS AND RELATED COMPOSITIONS OF DIFFERENTIATED CELLS,” the contents of each of which are incorporated by reference herein in their entirety for all purposes.

### FIELD

The present disclosure relates to methods for classifying the differentiation state of an in vitro population of cells, for instance an in vitro population of neuronal cells, as well as methods for selecting and/or implanting an in vitro population of cells having a desired differentiation state. Also provided herein are computing devices for performing the provided methods as well as related compositions, articles of manufacture, and kits, including for use in methods of treating a subject having a disease or condition, such as a neurodegenerative disease, for instance Parkinson's disease.

### BACKGROUND

Various methods for differentiating pluripotent stem cells into lineage specific cell populations, as well as the resulting cellular compositions, are contemplated for use in cell replacement therapies for patients with diseases resulting in a loss of function of a defined cell population. In some aspects, it is desirable to administer cells that have particular differentiation states. Improved methods of classifying and identifying said cells are needed.

### SUMMARY

Provided herein in some embodiments is a computing device for classifying the differentiation state of an in vitro population of cells, the device comprising a memory that comprises: a first reference dataset that comprises a representation of gene expression levels for one or more genes that are differentially expressed between cells at a first differentiation state and cells at a second differentiation state; and a second reference dataset that comprises a representation of gene expression levels for one or more genes that are differentially expressed between cells at the second differentiation state and cells at a third differentiation state.

In some of any of the provided embodiments, the computing device further comprises a processor that implements instructions stored in the memory to perform a method comprising: (a) receiving as input a test dataset that comprises expression levels for genes that are expressed in one or more test cells comprised in an in vitro population of cells, wherein the expression levels in the test dataset comprise expression levels for (i) one or more of the genes for which a representation of expression levels are included in the first reference dataset, and (ii) one or more of the genes for which a representation of expression levels are included in the second reference dataset; (b) calculating, using the test dataset and the first reference dataset, a first similarity score indicating whether the differentiation state of the test cells is more similar to the first differentiation state or to the second differentiation state; (c) calculating, using the test dataset and the second reference dataset, a second similarity score indicating whether the differentiation state of the test cells is more similar to the second differentiation state or to the third differentiation state; and (d) classifying the differentiation state of the one or more test cells based on one or both of the first similarity score and the second similarity score.

In some embodiments, the classifying is based on one of the first similarity score and the second similarity score. In some embodiments, the classifying is based on the lower of the first similarity score and the second similarity score. In some embodiments, the classifying is based on the higher of the first similarity score and the second similarity score. In some embodiments, the classifying is based on the first similarity score. In some embodiments, the classifying is based on the second similarity score.

In some embodiments, the classifying is based on both the first similarity score and the second similarity score.

In some of any of the provided embodiments, the computing device further comprises a processor that implements instructions stored in the memory to perform a method comprising: (a) receiving as input a test dataset that comprises expression levels for genes that are expressed in one or more test cells comprised in an in vitro population of cells, wherein the expression levels in the test dataset comprise expression levels for (i) one or more of the genes for which a representation of expression levels are included in the first reference dataset, and (ii) one or more of the genes for which a representation of expression levels are included in the second reference dataset; (b) calculating, using the test dataset and the first reference dataset, a first similarity score indicating whether the differentiation state of the test cells is more similar to the first differentiation state or to the second differentiation state; (c) calculating, using the test dataset and the second reference dataset, a second similarity score indicating whether the differentiation state of the test cells is more similar to the second differentiation state or to the third differentiation state; and (d) classifying the differentiation state of the one or more test cells based on the first similarity score and the second similarity score.

In some of any of the provided embodiments, the memory further comprises a control dataset that comprises a representation of gene expression levels for one or more genes that are expressed in cells at one or more control differentiation states, which control differentiation state may be the same as or different than one of the first, second, or third differentiation states. In some of any of the provided embodiments, the test dataset comprises gene expression levels for one or more of the genes for which a representation of expression levels are included in the control dataset; the instructions comprise calculating a degree of correlation between the representation of gene expression levels for one or more genes in the control dataset and gene expression levels for the one or more genes in the test dataset to calculate a correlation score; and the classifying the differentiation state of the one or more test cells is based on the correlation score and one or both of the first similarity score and the second similarity score.

In some embodiments, the classifying is based on the correlation score and one of the first similarity score and the second similarity score. In some embodiments, the classifying is based on the correlation score and the lower of the first similarity score and the second similarity score. In some embodiments, the classifying is based on the correlation score and the higher of the first similarity score and the second similarity score. In some embodiments, the classifying is based on the correlation score and the first similarity score. In some embodiments, the classifying is based on the correlation score and the second similarity score.

In some embodiments, the classifying is based on the correlation score and both the first similarity score and the second similarity score.

In some of any of the provided embodiments, the memory further comprises a control dataset that comprises a representation of gene expression levels for one or more genes that are expressed in cells at one or more control differentiation states, which control differentiation state may be the same as or different than one of the first, second, or third differentiation states. In some of any of the provided embodiments, the test dataset comprises gene expression levels for one or more of the genes for which a representation of expression levels are included in the control dataset; the instructions comprise calculating a degree of correlation between the representation of gene expression levels for one or more genes in the control dataset and gene expression levels for the one or more genes in the test dataset to calculate a correlation score; and the classifying the differentiation state of the one or more test cells is based on the first similarity score, the second similarity score, and the correlation score.

In some of any of the provided embodiments, the correlation score is calculated prior to calculating the first similarity score and the second similarity score, and the method is terminated if the correlation score for the test cells does not meet a predefined cutoff value.

In some of any of the provided embodiments, the control dataset comprises gene expression levels that are normalized by counts per million mapped reads (CPM) and filtered to include only gene expression levels that exceed a threshold CPM value. In some of any of the provided embodiments, the control dataset comprises a centroid of gene expression levels of the one or more genes in the control dataset. In some of any of the provided embodiments, the correlation score is calculated by normalizing the gene expression levels of the one or more genes in the test dataset and calculating a correlation of the gene expression levels of the one or more genes in the test dataset to the centroid. In some of any of the provided embodiments, the control dataset comprises coefficient of variation (CV) values of gene expression levels of the one or more genes in the control dataset, and the correlation to the centroid is weighted by the inverse of the CV values.

In some of any of the provided embodiments, the in vitro population of cells is from a culture of cells differentiated from pluripotent cells that are subjected to suitable differentiation conditions. In some of any of the provided embodiments, the first differentiation state is earlier in a stem cell differentiation pathway than the second differentiation state. In some of any of the provided embodiments, the second differentiation state is earlier in a stem cell differentiation pathway than the third differentiation state. In some of any of the provided embodiments, the first differentiation state is in a cell differentiation pathway that is parallel to a cell differentiation pathway of the second differentiation state.

In some of any of the provided embodiments, the population of cells are selected from the group consisting of stem-cell derived cardiac muscle cells, stem-cell derived skeletal muscle cells, stem-cell derived kidney tubule cells, stem-cell derived red blood cell cells, stem-cell derived smooth muscle cells, stem-cell derived lung cells, stem-cell derived thyroid cells, stem-cell derived pancreatic cells, stem-cell derived epidermal cells, stem-cell derived pigment cells, and stem-cell derived neuronal cells. In some of any of the provided embodiments, the population of cells are stem-cell derived neuronal cells. In some of any of the provided embodiments, the second differentiation state is the differentiation state of a determined dopaminergic neuronal cell. In some of any of the provided embodiments, the second differentiation state is the differentiation state of cells with fitness for engraftment.

In some of any of the provided embodiments, the second differentiation state is the differentiation state of a hematopoietic progenitor cell.

In some of any of the provided embodiments, the first reference dataset comprises a representation of gene expression levels for one or more genes selected from Table E1. In some of any of the provided embodiments, the second reference dataset comprises a representation of gene expression levels for one or more genes selected from Table E2. In some of any of the provided embodiments, the first reference dataset comprises a representation of gene expression levels for at least 20 genes selected from Table E1. In some of any of the provided embodiments, the second reference dataset comprises a representation of gene expression levels for at least 20 genes selected from Table E2. In some of any of the provided embodiments, the first reference dataset comprises a representation of gene expression levels for at least 50 genes selected from Table E1. In some of any of the provided embodiments, the second reference dataset comprises a representation of gene expression levels for at least 50 genes selected from Table E2.

In some of any of the provided embodiments, at least one of the first, second and third differentiation states is characterized using an in vitro assay. In some of any of the provided embodiments, at least one of the first, second and third differentiation states is characterized using an in vivo assay. In some of any of the provided embodiments, the in vivo assay comprises determining whether reference cells are capable of surviving, engrafting, and/or innervating tissue when administered to an animal or human subject. In some of any of the provided embodiments, the in vivo assay comprises determining whether reference cells ameliorate or reverse symptoms of a neurodegenerative disease when implanted into an animal or human subject.

In some of any of the provided embodiments, the animal subject comprises an animal model of Parkinson's disease. In some of any of the provided embodiments, the memory further comprises one or more additional reference datasets, wherein each of the additional reference datasets comprises a representation of gene expression levels for one or more genes that are differentially expressed between cells at the second differentiation state and cells at an additional differentiation state, wherein: the processor implements instructions to calculate, using the additional reference datasets, one or more additional similarity scores indicating whether the differentiation state of the test cells is more similar to the second differentiation state or to one of the one or more additional differentiation states, and the classifying the differentiation state of the one or more test cells is based on the first similarity score, the second similarity score, and the one or more additional similarity scores.

In some of any of the provided embodiments, the representations of gene expression levels in the first reference dataset and/or the second reference dataset are obtained using machine learning. In some of any of the provided embodiments, the machine learning comprises principal component analysis. In some of any of the provided embodiments, the representations of gene expression levels in the first reference dataset and/or the second reference dataset comprise normalized gene expression levels. In some of any of the provided embodiments, the differentiation state of the one or more test cells is classified as being the second differentiation state if the first and second similarity scores indicate that the differentiation state of the one or more test cells is more similar to the second differentiation state. In some of any of the provided embodiments, the differentiation state of the one or more test cells is classified as being the second differentiation state if the first similarity score indicates that the differentiation state of the one or more test cells is more similar to the second differentiation state. In some of any of the provided embodiments, the differentiation state of the one or more test cells is classified as being the second differentiation state if the second similarity score indicates that the differentiation state of the one or more test cells is more similar to the second differentiation state.

Also provided herein in some embodiments is a method for selecting a population of cells having a desired differentiation state, the method comprising: (a) calculating a first similarity score using a test dataset and a first reference dataset, wherein: the first reference dataset comprises a representation of gene expression levels for one or more genes that are differentially expressed between cells at a first differentiation state and cells at a second differentiation state, the test dataset comprises expression levels for genes that are expressed in one or more test cells comprised in an in vitro population of cells, wherein the expression levels in the test dataset comprise expression levels for one or more of the genes for which a representation of expression levels are included in the first reference dataset, and the first similarity score indicates whether the differentiation state of the test cells is more similar to the first differentiation state or to the second differentiation state; (b) calculating a second similarity score using the test dataset and a second reference dataset, wherein: the second reference dataset comprises a representation of gene expression levels for one or more genes that are differentially expressed between cells at the second differentiation state and cells at a third differentiation state, the expression levels in the test dataset comprise expression levels for one or more of the genes for which a representation of expression levels are included in the second reference dataset, and the second similarity score indicates whether the differentiation state of the test cells is more similar to the second differentiation state or to the third differentiation state; and (c) classifying the differentiation state of the one or more test cells based on one or both of the first similarity score and the second similarity score.

In some embodiments, the classifying is based on one of the first similarity score and the second similarity score. In some embodiments, the classifying is based on the lower of the first similarity score and the second similarity score. In some embodiments, the classifying is based on the higher of the first similarity score and the second similarity score. In some embodiments, the classifying is based on the first similarity score. In some embodiments, the classifying is based on the second similarity score.

In some embodiments, the classifying is based on both the first similarity score and the second similarity score.

Also provided herein in some embodiments is a method for selecting a population of cells having a desired differentiation state, the method comprising: (a) calculating a first similarity score using a test dataset and a first reference dataset, wherein: the first reference dataset comprises a representation of gene expression levels for one or more genes that are differentially expressed between cells at a first differentiation state and cells at a second differentiation state, the test dataset comprises expression levels for genes that are expressed in one or more test cells comprised in an in vitro population of cells, wherein the expression levels in the test dataset comprise expression levels for one or more of the genes for which a representation of expression levels are included in the first reference dataset, and the first similarity score indicates whether the differentiation state of the test cells is more similar to the first differentiation state or to the second differentiation state; (b) calculating a second similarity score using the test dataset and a second reference dataset, wherein: the second reference dataset comprises a representation of gene expression levels for one or more genes that are differentially expressed between cells at the second differentiation state and cells at a third differentiation state, the expression levels in the test dataset comprise expression levels for one or more of the genes for which a representation of expression levels are included in the second reference dataset, and the second similarity score indicates whether the differentiation state of the test cells is more similar to the second differentiation state or to the third differentiation state; and (c) classifying the differentiation state of the one or more test cells based on the first similarity score and the second similarity score.

In some of any of the provided embodiments, the test dataset comprises gene expression levels for one or more genes for which a representation of expression levels are included in a control dataset that comprises a representation of gene expression levels for one or more genes that are expressed in cells at a control differentiation state, which control differentiation state may be the same as or different than one of the first, second, or third differentiation states; the method further comprises calculating a degree of correlation between the representation of gene expression levels for one or more genes in the control dataset and gene expression levels for the one or more genes in the test dataset to calculate a correlation score; and the classifying the differentiation state of the one or more test cells is based on the correlation score and one or both of the first similarity score and the second similarity score.

In some embodiments, the classifying is based on the correlation score and one of the first similarity score and the second similarity score. In some embodiments, the classifying is based on the correlation score and the lower of the first similarity score and the second similarity score. In some embodiments, the classifying is based on the correlation score and the higher of the first similarity score and the second similarity score. In some embodiments, the classifying is based on the correlation score and the first similarity score. In some embodiments, the classifying is based on the correlation score and the second similarity score.

In some embodiments, the classifying is based on the correlation score and both the first similarity score and the second similarity score.

In some of any of the provided embodiments, the test dataset comprises gene expression levels for one or more genes for which a representation of expression levels are included in a control dataset that comprises a representation of gene expression levels for one or more genes that are expressed in cells at a control differentiation state, which control differentiation state may be the same as or different than one of the first, second, or third differentiation states; the method further comprises calculating a degree of correlation between the representation of gene expression levels for one or more genes in the control dataset and gene expression levels for the one or more genes in the test dataset to calculate a correlation score; and the classifying the differentiation state of the one or more test cells is based on the first similarity score, the second similarity score, and the correlation score.

In some of any of the provided embodiments, the correlation score is calculated prior to calculating the first similarity score and the second similarity score and the method is terminated if the correlation score for the test cells does not meet a predefined cutoff value.

In some of any of the provided embodiments, the control dataset comprises gene expression levels that are normalized by counts per million mapped reads (CPM) and filtered to include only gene expression levels that exceed a threshold CPM value. In some of any of the provided embodiments, the control dataset comprises a centroid of gene expression levels of the one or more genes in the control dataset. In some of any of the provided embodiments, the correlation score is calculated by normalizing the gene expression levels of the one or more genes in the test dataset and calculating a correlation of the gene expression levels of the one or more genes in the test dataset to the centroid. In some of any of the provided embodiments, the control dataset comprises coefficient of variation (CV) values of gene expression levels of the one or more genes in the control dataset, and the correlation to the centroid is weighted by the inverse of the CV values.

In some of any of the provided embodiments, the first differentiation state is earlier in a stem cell differentiation pathway than the second differentiation state. In some of any of the provided embodiments, the second differentiation state is earlier in a stem cell differentiation pathway than the third differentiation state. In some of any of the provided embodiments, the first differentiation state is in a cell differentiation pathway that is parallel to a cell differentiation pathway of the second differentiation state.

In some of any of the provided embodiments, the population of cells are selected from the group consisting of stem-cell derived cardiac muscle cells, stem-cell derived skeletal muscle cells, stem-cell derived kidney tubule cells, stem-cell derived red blood cell cells, stem-cell derived smooth muscle cells, stem-cell derived lung cells, stem-cell derived thyroid cells, stem-cell derived pancreatic cells, stem-cell derived epidermal cells, stem-cell derived pigment cells, and stem-cell derived neuronal cells. In some of any of the provided embodiments, the population of cells are stem-cell derived neuronal cells.

In some of any of the provided embodiments, the second differentiation state is the differentiation state of a determined dopaminergic neuronal cell. In some of any of the provided embodiments, the second differentiation state is the differentiation state of cells with fitness for engraftment.

In some of any of the provided embodiments, the second differentiation state is the differentiation state of a hematopoietic progenitor cell.

In some of any of the provided embodiments, the first reference dataset comprises a representation of gene expression levels for one or more genes selected from Table E1. In some of any of the provided embodiments, the second reference dataset comprises a representation of gene expression levels for one or more genes selected from Table E2. In some of any of the provided embodiments, the first reference dataset comprises a representation of gene expression levels for at least 20 genes selected from Table E1. In some of any of the provided embodiments, the second reference dataset comprises a representation of gene expression levels for at least 20 genes selected from Table E2. In some of any of the provided embodiments, the first reference dataset comprises a representation of gene expression levels for at least 50 genes selected from Table E1. In some of any of the provided embodiments, the second reference dataset comprises a representation of gene expression levels for at least 50 genes selected from Table E2.

In some of any of the provided embodiments, at least one of the first, second and third differentiation states is characterized using an in vivo assay. In some of any of the provided embodiments, the in vivo assay comprises determining whether reference cells are capable of surviving, engrafting, and/or innervating tissue when administered to an animal or human subject.

In some of any of the provided embodiments, the in vivo assay comprises determining whether reference cells ameliorate or reverse symptoms of a neurodegenerative disease when implanted into an animal or human subject. In some of any of the provided embodiments, the animal subject comprises an animal model of Parkinson's disease. In some of any of the provided embodiments, the method further comprises calculating one or more additional similarity scores using one or more additional reference datasets, wherein: each of the additional reference datasets comprises a representation of gene expression levels for one or more genes that are differentially expressed between cells at the second differentiation state and cells at an additional differentiation state; the one or more additional similarity scores indicate whether the differentiation state of the test cells is more similar to the second differentiation state or to one of the one or more additional differentiation states, and the classifying the differentiation state of the one or more test cells is based on the first similarity score, the second similarity score, and the one or more additional similarity scores.

In some of any of the provided embodiments, the representations of gene expression levels in the first reference dataset and/or the second reference dataset are obtained using machine learning. In some of any of the provided embodiments, the machine learning comprises principal component analysis. In some of any of the provided embodiments, the representations of gene expression levels in the first reference dataset and/or the second reference dataset comprise normalized gene expression levels.

In some of any of the provided embodiments, the method further comprises classifying the differentiation state of the one or more test cells as being the second differentiation state if the first similarity score indicates that the differentiation state of the one or more test cells is more similar to the second differentiation state. In some of any of the provided embodiments, the method further comprises classifying the differentiation state of the one or more test cells as being the second differentiation state if the second similarity score indicates that the differentiation state of the one or more test cells is more similar to the second differentiation state. In some of any of the provided embodiments, the method further comprises selecting the in vitro population of cells comprising one or more test cells classified as having the second differentiation state as having the desired differentiation state.

In some of any of the provided embodiments, the method further comprises classifying the differentiation state of the one or more test cells as being the second differentiation state if the first and second similarity scores indicate that the differentiation state of the one or more test cells is more similar to the second differentiation state. In some of any of the provided embodiments, the method further comprises selecting the in vitro population of cells comprising one or more test cells classified as having the second differentiation state as having the desired differentiation state.

Also provided herein in some embodiments is a method for selecting a population of cells having a desired differentiation state, comprising (a) obtaining a test dataset comprising gene expression levels of one or more genes selected from AC010247.2, ANKRD33B, APC2, AQP4, ASCL1, AURKB, BARHL2, CACNA1G, CAPN6, CBLN1, CCNB2, CDH1, CDH20, CHGA, COL1A1, COL1A2, COL22A1, COL4A1, CRABP1, DBX1, DCN, DCX, DDC, DOCK10, E2F4, EDNRB, ESRP1, EZH2, FABP7, FBLN1, FLRT3, FOXA2, FOXM1, GAP43, GFAP, GFRA1, GJA1, GLRA2, HES1, HES2, HES5, ITGA5, JPH4, LDHA, LIN28A, LIX1, LMX1A, LUM, NCAM1, NES, NEUROG2, NGFR, NKX2-2, NMNAT2, NPTX1, NR4A2, NR4A2 (NURR1), NSG2, NFYA, OLFM3, OLIG1, OLIG2, OTX2, P4HA1, PBX1, PDGFRA, PIEZO2, PITX3, PLP1, PMEL, PMP2, POSTN, POU2F2, PPP2R2B, PRTG, PTTG1, REST, RET, RFX4, RFX4, SALL4, SIN3A, SLC16A3, SLC18A2, SLC1A, SLC1A2, SLC1A3, SLC4A4, SMAD4, SNAP25, SOX10, SOX2, SOX9, STMN2, SUZ12, SV2B, SYN1, SYT1, SYT13, TH, TOP2A, TPH1, TPM2, and TXNIP for one or more test cells comprised in an in vitro population of cells; and (b) applying the gene expression levels as input to a process configured to predict if the population of cells has a desired differentiation state.

In some of any of the provided embodiments, the in vitro population of cells comprises stem-cell derived neuronal cells. In some of any of the provided embodiments, the desired differentiation state is the differentiation state of a determined dopaminergic neuronal cell. In some of any of the provided embodiments, the desired differentiation state is the differentiation state of cells with fitness for engraftment.

In some of any of the provided embodiments, the desired differentiation state is the differentiation state of a hematopoietic progenitor cell.

Also provided herein in some embodiments is a method for selecting a population of cells predicted to exhibit neurite outgrowth following implantation in a brain region, comprising (a) obtaining a test dataset comprising gene expression levels of one or more genes selected from AC010247.2, ANKRD33B, APC2, AQP4, ASCL1, AURKB, BARHL2, CACNA1G, CAPN6, CBLN1, CCNB2, CDH1, CDH20, CHGA, COL1A1, COL1A2, COL22A1, COL4A1, CRABP1, DBX1, DCN, DCX, DDC, DOCK10, E2F4, EDNRB, ESRP1, EZH2, FABP7, FBLN1, FLRT3, FOXA2, FOXM1, GAP43, GFAP, GFRA1, GJA1, GLRA2, HES1, HES2, HES5, ITGA5, JPH4, LDHA, LIN28A, LIX1, LMX1A, LUM, NCAM1, NES, NEUROG2, NGFR, NKX2-2, NMNAT2, NPTX1, NR4A2, NR4A2 (NURR1), NSG2, NFYA, OLFM3, OLIG1, OLIG2, OTX2, P4HA1, PBX1, PDGFRA, PIEZO2, PITX3, PLP1, PMEL, PMP2, POSTN, POU2F2, PPP2R2B, PRTG, PTTG1, REST, RET, RFX4, RFX4, SALL4, SIN3A, SLC16A3, SLC18A2, SLC1A, SLC1A2, SLC1A3, SLC4A4, SMAD4, SNAP25, SOX10, SOX2, SOX9, STMN2, SUZ12, SV2B, SYN1, SYT1, SYT13, TH, TOP2A, TPH1, TPM2, and TXNIP for one or more test cells comprised in an in vitro population of cells; and (b) applying the gene expression levels as input to a process configured to predict if the population of cells will exhibit neurite outgrowth following implantation in a brain region.

In some of any of the provided embodiments, the one or more genes comprise at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95 or more of AC010247.2, ANKRD33B, APC2, AQP4, ASCL1, AURKB, BARHL2, CACNA1G, CAPN6, CBLN1, CCNB2, CDH1, CDH20, CHGA, COL1A1, COL1A2, COL22A1, COL4A1, CRABP1, DBX1, DCN, DCX, DDC, DOCK10, E2F4, EDNRB, ESRP1, EZH2, FABP7, FBLN1, FLRT3, FOXA2, FOXM1, GAP43, GFAP, GFRA1, GJA1, GLRA2, HES1, HES2, HES5, ITGA5, JPH4, LDHA, LIN28A, LIX1, LMX1A, LUM, NCAM1, NES, NEUROG2, NGFR, NKX2-2, NMNAT2, NPTX1, NR4A2, NR4A2 (NURR1), NSG2, NFYA, OLFM3, OLIG1, OLIG2, OTX2, P4HA1, PBX1, PDGFRA, PIEZO2, PITX3, PLP1, PMEL, PMP2, POSTN, POU2F2, PPP2R2B, PRTG, PTTG1, REST, RET, RFX4, RFX4, SALL4, SIN3A, SLC16A3, SLC18A2, SLC1A, SLC1A2, SLC1A3, SLC4A4, SMAD4, SNAP25, SOX10, SOX2, SOX9, STMN2, SUZ12, SV2B, SYN1, SYT1, SYT13, TH, TOP2A, TPH1, TPM2, and TXNIP.

In some of any of the provided embodiments, the process comprises a machine learning model. In some of any of the provided embodiments, the machine learning model has been trained using gene expression levels of the one or more genes. In some of any of the provided embodiments, one or more outputs of the machine learning model are used to predict if the population of cells have the desired differentiation state. In some of any of the provided embodiments, one or more outputs of the machine learning model are used to predict if the population of cells will exhibit neurite outgrowth following implantation in a brain region. In some of any of the provided embodiments, the method further comprises classifying the differentiation state of the one or more test cells based on one or more outputs of the machine learning model. In some of any of the provided embodiments, the method further comprises predicting if the test cells will exhibit neurite outgrowth following implantation in a brain region based on one or more outputs of the machine learning model. In some of any of the provided embodiments, the method further comprises selecting the in vitro population of cells comprising one or more test cells classified as having the desired differentiation state. In some of any of the provided embodiments, the method further comprises selecting the in vitro population of cells comprising one or more test cells predicted to exhibit neurite outgrowth following implantation in a brain region.

Also provided herein in some embodiments is a method for implanting a population of cells having a desired differentiation state into a subject, the method comprising: (a) selecting a population of cells having a desired differentiation state using the any of the provided methods; and (b) implanting the population of cells into a subject. In some of any of the provided embodiments, the cells having the desired differentiation state are determined dopaminergic cells, and the population of cells is implanted into a brain region of the subject. In some of any of the provided embodiments, the cells having the desired differentiation state are from a culture of cells differentiated from pluripotent cells under conditions to neurally differentiate the cells.

In some of any of the provided embodiments, the cells having the desired differentiation state are hematopoietic progenitor cells, and the population of cells is implanted into a brain region of the subject. In some of any of the provided embodiments, the cells having the desired differentiation state are from a culture of cells differentiated from pluripotent cells under conditions to neurally differentiate the cells.

Also provided herein in some embodiments is a pharmaceutical composition comprising a pharmaceutical carrier and a population of cells having a desired differentiation state, wherein the cells are selected using any of the provided methods.

In some of any of the provided embodiments, the cells having the desired differentiation state are neuronal cells that are suitable for treatment of a neurodegenerative disease when implanted into a brain of a subject in need of such treatment. In some of any of the provided embodiments, the neuronal cells comprise determined dopaminergic cells. In some of any of the provided embodiments, the neuronal cells comprise engraftment-capable neuronal cells.

In some of any of the provided embodiments, the neuronal cells comprise hematopoietic progenitor cells.

Also provided herein in some embodiments is a method for training a machine learning model classifying the differentiation state of an in vitro population of cells, the method comprising: (a) obtaining, for a plurality of reference populations of cells, gene expression levels for one or more genes that are differentially expressed between cells at a first differentiation state and cells at a second differentiation state and applying the gene expression levels as input to train a first machine learning model to predict if an in vitro population of cells comprises one or more test cells having a differentiation state that is more similar to the first differentiation state or to the second differentiation state; and (b) obtaining, for a plurality of reference populations of cells, gene expression levels for one or more genes that are differentially expressed between cells at the second differentiation state and cells at a third differentiation state and applying the gene expression levels as input to train a second machine learning model to predict if an in vitro population of cells comprises one or more test cells having a differentiation state that is more similar to the second differentiation state or to the third differentiation state.

Also provided herein in some embodiments is a method for training a machine learning model classifying the differentiation state of an in vitro population of cells, the method comprising: (a) selecting one or more genes that are differentially expressed between cells at a first differentiation state and cells at a second differentiation state and applying expression levels of the selected genes for a plurality of reference populations of cells as input to train a first machine learning model to predict if an in vitro population of cells comprises one or more test cells having a differentiation state that is more similar to the first differentiation state or to the second differentiation state; and (c) selecting one or more genes that are differentially expressed between cells at the second differentiation state and cells at a third differentiation state and applying expression levels of the selected genes for a plurality of reference populations of cells as input to train a second machine learning model to predict if an in vitro population of cells comprises one or more test cells having a differentiation state that is more similar to the second differentiation state or to the third differentiation state.

In some of any of the provided embodiments, the method further comprises obtaining gene expression levels for one or more genes that are expressed in cells at a control differentiation state, which control differentiation state may be the same as or different than one of the first, second, or third differentiation states, and applying the gene expression levels as input to train a control machine learning model to predict if an in vitro population of cells comprises one or more test cells that are similar to the cells at the control differentiation state.

Also provided herein is a pharmaceutical composition comprising a pharmaceutical carrier and a population of neuronal cells, wherein the cells are selected using any of the provided methods.

Also provided herein is an in vitro stem cell-derived neuronal cell population comprising cells that express one or more genes selected from the group consisting of CCNB2, AURKB, PTTG1, TOP2A, NEUROG2, HES1, REST, E2F4, FOXM1, SIN3A, NFYA, LIN28A, FLRT3, ITGA5, NES, SOX2, SOX9, and RFX4. In some embodiments, the in vitro stem-cell derived neuronal cell population is one in which: (1) at least one gene from the one or more genes is selected from the group consisting of CCNB2, AURKB, PTTG1, TOP2A, NEUROG2, HES1, REST, E2F4, FOXM1, SIN3A, NFYA, LIN28A, FLRT3, and ITGA5; and (2) at least one gene from the one or more genes is selected from the group consisting of NES, SOX2, SOX9, and RFX4. In some embodiments, at least one of the one or more genes is REST.

In some of any embodiments of the in vitro stem-cell derived neuronal cell population, at least 50% of cells within the population express the one or more genes.

In some of any embodiments of the in vitro stem-cell derived neuronal cell population, at least 60% of cells within the population express the one or more genes.

In some of any embodiments of the in vitro stem-cell derived neuronal cell population, at least 70% of cells within the population express the one or more genes.

In some of any embodiments of the in vitro stem-cell derived neuronal cell population, at least 80% of cells within the population express the one or more genes.

In some of any embodiments of the in vitro stem-cell derived neuronal cell population, at least 90% of cells within the population express the one or more genes.

In some of any embodiments of the in vitro stem-cell derived neuronal cell population, cells in the population express EN1 and CORIN. In some embodiments, less than 20% of the total cells in the composition express TH. In some embodiments, less than 10% of the total cells in the composition express TH.

In some of any embodiments of the in vitro stem-cell derived neuronal cell population, the expression is RNA expression. In some embodiments, the RNA expression is measured by RNA sequencing.

In some of any embodiments of the in vitro stem-cell derived neuronal cell population, the population has been differentiated in vitro from a pluripotent stem cell (PSC).

In some of any embodiments of the in vitro stem-cell derived neuronal cell population, the one or more genes is a gene that is overexpressed in cells of the population compared to the iPSCs. In some embodiments, one or more gene is a gene that is overexpressed in cells of the population compared to cells of a precursor population differentiated from the iPSCs. In some embodiments, one or more gene is a gene that is overexpressed in cells of the population compared to cells of a mature committed dopaminergic neuronal cell population differentiated from the iPSCs. In some embodiments, the mature committed dopaminergic neuronal cells express LMX1A and/or NR4A2 (NURR1). In some embodiments, among cells in the committed dopaminergic neuronal cell population, at least 40%, at least 50%, at least 60%, at least 70%, or at least 80% of the cells express LMX1A and/or NR4A2. In some embodiments, the overexpression is a positive log 2 fold change of greater than or greater than about 1.5-fold, 2.0-fold, 3.0-fold, 4.0-fold or 5-fold.

In some of any embodiments of the in vitro stem-cell derived neuronal cell population, the one or more genes is a gene that is reduced in expression in cells of the population compared to the iPSCs. In some embodiments, one or more gene is a gene that is reduced in expression in cells of the population compared to cells of a precursor population differentiated from the iPSCs. In some embodiments, the one or more genes is a gene that is reduced in expression in cells of the population compared to cells of a mature committed dopaminergic neuronal cell population differentiated from the iPSCs. In some embodiments, the mature committed dopaminergic neuronal cells express LMX1A and/or NR4A2 (NURR1). In some embodiments, among cells in the committed dopaminergic neuronal cell population, at least 40%, at least 50%, at least 60%, at least 70%, or at least 80% of the cells express LMX1A and/or NR4A2. In some embodiments, the reduced expression is a negative log 2 fold change of greater than or greater than about 1.5-fold, 2.0-fold, 3.0-fold, 4.0-fold or 5-fold.

In some of any embodiments of the in vitro stem-cell derived neuronal cell population, less than 30%, less than 20%, or less than 10% of the cells in the population express LMX1A and/or NR4A2.

In some of any embodiments of the in vitro stem-cell derived neuronal cell population, cells in the population are capable of engrafting in and innervating other cells in vivo. In some embodiments, cells in the population are capable of exhibiting neurite outgrowth when administered to the brain of a subject. In some embodiments, cells in the population are capable of producing dopamine and optionally do not produce or do not substantially produce norepinephrine.

In some of any embodiments of the in vitro stem-cell derived neuronal cell population, the population comprises at least 5 million total cells, at least 10 million total cells, at least 15 million total cells, at least 20 million total cells, at least 30 million total cells, at least 40 million total cells, at least 50 million total cells, at least 100 million total cells, at least 150 million total cells, or at least 200 million total cells. In some embodiments, the population comprises between at or about 5 million total cells and at or about 200 million total cells, between at or about 5 million total cells and at or about 150 million total cells, between at or about 5 million total cells and at or about 100 million total cells, between at or about 5 million total cells and at or about 50 million total cells, between at or about 5 million total cells and at or about 25 million total cells, between at or about 5 million total cells and at or about 10 million total cells, between at or about 10 million total cells and at or about 200 million total cells, between at or about 10 million total cells and at or about 150 million total cells, between at or about 10 million total cells and at or about 100 million total cells, between at or about 10 million total cells and at or about 50 million total cells, between at or about 10 million total cells and at or about 25 million total cells, between at or about 25 million total cells and at or about 200 million total cells, between at or about 25 million total cells and at or about 150 million total cells, between at or about 25 million total cells and at or about 100 million total cells, between at or about 25 million total cells and at or about 50 million total cells, between at or about 50 million total cells and at or about 200 million total cells, between at or about 50 million total cells and at or about 150 million total cells, between at or about 50 million total cells and at or about 100 million total cells, between at or about 100 million total cells and at or about 200 million total cells, between at or about 100 million total cells and at or about 150 million total cells, or between at or about 150 million total cells and at or about 200 million total cells.

In some of any embodiments of the in vitro stem-cell derived neuronal cell population, at least about 70%, 75%, 80%, 85%, 90%, or 95% of the total cells in the composition are viable.

Also provided herein is a pharmaceutical composition comprising a pharmaceutical carrier and an in vitro stem-cell derived neuronal cell population as provided herein.

In embodiments of any of the provided pharmaceutical compositions, the composition comprises a cryoprotectant. In some embodiments, the cryoprotectant is selected from among the group consisting of glycerol, propylene glycol, and dimethyl sulfoxide (DMSO).

In embodiments of any of the provided pharmaceutical compositions, the composition is for use in treatment of a neurodegenerative disease or condition in a subject, optionally wherein the neurodegenerative disease or condition comprises a loss of dopaminergic neurons. In some embodiments, the neurodegenerative disease or condition comprises a loss of dopaminergic neurons in the substantia nigra, optionally in the SNc. In some embodiments, the neurodegenerative disease or condition is Parkinson's disease. In some embodiments, the neurodegenerative disease or condition is a Parkinsonism.

In embodiments of any of the provided pharmaceutical compositions, the composition is for use in treatment of a neurodegenerative disease or condition in a subject, wherein the neurodegenerative disease or condition comprises a loss of microglial cells. In some embodiments, the neurodegenerative disease or condition is Parkinson's disease. In some embodiments, the neurodegenerative disease or condition is a Parkinsonism. In some embodiments, the neurodegenerative disease or condition is an age-related neurodegenerative disease. In some embodiments, the neurodegenerative disease or condition is Alzheimer's disease. In some embodiments, the neurodegenerative disease or condition is frontotemporal dementia.

Also provided herein is a method of treatment, comprising implanting in a brain region of a subject in need thereof a therapeutically effective amount of any of the provided pharmaceutical compositions. In some embodiments, the number of cells implanted in the subject is between about 0.25×10⁶ cells and about 20×10⁶ cells, between about 0.25×10⁶ cells and about 15×10⁶ cells, between about 0.25×10⁶ cells and about 10×10⁶ cells, between about 0.25×10⁶ cells and about 5×10⁶ cells, between about 0.25×10⁶ cells and about 1×10⁶ cells, between about 0.25×10⁶ cells and about 0.75×10⁶ cells, between about 0.25×10⁶ cells and about 0.5×10⁶ cells, between about 0.5×10⁶ cells and about 20×10⁶ cells, between about 0.5×10⁶ cells and about 15×10⁶ cells, between about 0.5×10⁶ cells and about 10×10⁶ cells, between about 0.5×10⁶ cells and about 5×10⁶ cells, between about 0.5×10⁶ cells and about 1×10⁶ cells, between about 0.5×10⁶ cells and about 0.75×10⁶ cells, between about 0.75×10⁶ cells and about 20×10⁶ cells, between about 0.75×10⁶ cells and about 15×10⁶ cells, between about 0.75×10⁶ cells and about 10×10⁶ cells, between about 0.75×10⁶ cells and about 5×10⁶ cells, between about 0.75×10⁶ cells and about 1×10⁶ cells, between about 1×10⁶ cells and about 20×10⁶ cells, between about 1×10⁶ cells and about 15×10⁶ cells, between about 1×10⁶ cells and about 10×10⁶ cells, between about 1×10⁶ cells and about 5×10⁶ cells, between about 5×10⁶ cells and about 20×10⁶ cells, between about 5×10⁶ cells and about 15×10⁶ cells, between about 5×10⁶ cells and about 10×10⁶ cells, between about 10×10⁶ cells and about 20×10⁶ cells, between about 10×10⁶ cells and about 15×10⁶ cells, or between about 15×10⁶ cells and about 20×10⁶ cells.

In embodiments of any of the provided treatment methods, the subject has a neurodegenerative disease or condition. In some embodiments, the neurodegenerative disease or condition comprises the loss of dopaminergic neurons. In some embodiments, the subject has lost at least 50%, at least 60%, at least 70%, or at least 80% of dopaminergic neurons. In some embodiments, the subject has lost at least 50%, at least 60%, at least 70%, or at least 80% of dopaminergic neurons in the substantia nigra (SN), optionally in the SN pars compacta (SNc). In some embodiments, the neurodegenerative disease or condition is a Parkinsonism. In some embodiments, the neurodegenerative disease or condition is Parkinson's disease.

In embodiments of any of the provided treatment methods, the subject has a neurodegenerative disease or condition. In some embodiments, the neurodegenerative disease or condition comprises the loss of microglial cells. In some embodiments, the neurodegenerative disease or condition is a Parkinsonism. In some embodiments, the neurodegenerative disease or condition is Parkinson's disease. In some embodiments, the neurodegenerative disease or condition is an age-related neurodegenerative disease. In some embodiments, the neurodegenerative disease or condition is Alzheimer's disease. In some embodiments, the neurodegenerative disease or condition is frontotemporal dementia.

In embodiments of any of the provided methods, the implantation into a brain region is a brain region that is the substantia nigra. In some embodiments, the implanting is by stereotactic injection. In some embodiments, the cells of the pharmaceutical composition are autologous to the subject.

### BRIEF DESCRIPTION OF THE DRAWINGS

FIG. 1A shows a decision tree for an exemplary method of identifying a cell population at a desired differentiation state (e.g., an intermediate differentiation state, such as a determined state) using gene expression levels. In this exemplary method, gene expression levels of a test cell population are first assessed to determine if the expression levels resemble those of the reference cell populations used during method development. If the expression levels are not too dissimilar or novel, the expression levels are next assessed to determine if they are more consistent with those of a population of earlier-state cells (e.g., precursor cells) or with those of a population of intermediate-state cells (e.g., determined cells). If the expression levels are more consistent with those of a population of intermediate-state cells, the expression levels are finally assessed to determine if they are more consistent with those of a population of later-state cells (e.g., committed cells) or with those of a population of intermediate-state cells. If the gene expression levels are more consistent with those of a population of intermediate-state cells, the test population is identified as such.

FIG. 1B shows how the provided methods can be used to identify cells at an intermediate differentiation state, such as a determined state. As shown in FIG. 1B, the provided methods can be used for multiple target cell types and multiple in vitro differentiation protocols. Different differentiation protocols within the same target cell type can confer different optimal intermediate timings. This intermediate stage of differentiation could be when the cell population is most appropriate, for example, for transplantation, such as for the treatment of a disease or condition. As shown herein, methods that are trained with gene expression levels of cells from a first differentiation protocol can also be used for identifying cells at an intermediate differentiation state in a second differentiation protocol. Times in days (d) shown in FIG. 1B are for example only.

FIG. 2A and FIG. 2B show flowcharts for the training and use of an exemplary machine learning method for identifying a population of intermediate-state cells (e.g., determined cells) using gene expression levels. FIG. 2A shows flowcharts for determining a cutoff value for a novelty score indicating if gene expression levels of a test cell population resemble those of reference cell populations used for training the method and how this cutoff value can be applied for test cell populations. FIG. 2B shows flowcharts for training a first model that discriminates between early-state cells (e.g., precursor cells) and intermediate-state cells (e.g., determined cells; Model A); training a separate, second model that discriminates between later-state cells (e.g., committed cells) and intermediate-state cells (e.g., determined cells; Model B); and how both models can be applied to test cell populations. These procedures as applied to reference cell populations harvested at different time points during a neural differentiation protocol are described in Example 1.

FIG. 3A-3H show results for a machine learning method trained using reference cell populations harvested at different time points during a neural differentiation protocol. FIG. 3A-3F show results for neural cell populations. FIG. 3G shows results for glial test cell populations. FIG. 3H shows results for test cell populations of various cell types.

FIG. 4A-4D and FIG. 5A-5D show results for a machine learning method trained using reference cell populations harvested at different time points during a microglial differentiation protocol. FIG. 4A-4D show results for the reference cell populations. FIG. 5A-5D show validation results with test cell populations not used for model training.

### DETAILED DESCRIPTION

Provided herein in some embodiments are methods for classifying the differentiation state of a population of cells. Also provided herein in some embodiments are methods for selecting a population of cells having a desired differentiation state, for instance a population of cells classified by any of the provided methods as having the desired differentiation state. Also provided herein in some embodiments are methods for implanting a population of cells having a desired differentiation state, for instance a population of cells classified or selected according to any of the provided methods.

In some embodiments, the provided methods involve classifying the differentiation state of a population of cells. In some embodiments, the classifying is based on characteristics of one or more test cells of the population of cells. In some embodiments, the classifying is based on gene expression levels of the one or more test cells of the population of cells.

Also provided herein in some embodiments are methods for identifying a population of cells predicted to exhibit neurite outgrowth following implantation in a brain region. Also provided herein in some embodiments are methods for selecting a population of cells predicted to exhibit neurite outgrowth following implantation in a brain region, for instance a population of cells identified as such by any of the provided methods. Also provided herein in some embodiments are methods for implanting a population of cells predicted to exhibit neurite outgrowth following implantation in a brain region, for instance a population of cells identified or selected as such according to any of the provided methods.

In some embodiments, the population is an in vitro population of cells.

In some embodiments, the methods include steps for calculating a first similarity score and a second similarity score using the gene expression levels. In some embodiments, the classifying is based on one or both of the first and second similarity scores. In some embodiments, the classifying is based on one of the first similarity score and the second similarity score. In some embodiments, the classifying is based on the lower of the first similarity score and the second similarity score. In some embodiments, the classifying is based on the higher of the first similarity score and the second similarity score. In some embodiments, the classifying is based on the first similarity score. In some embodiments, the classifying is based on the second similarity score. In some embodiments, the first similarity score indicates whether the differentiation state of the population of cells is more similar to a first differentiation state or a second differentiation state. In some embodiments, the second similarity score indicates whether the differentiation state of the population of cells is more similar to the second differentiation state or a third differentiation state.

In some embodiments, the methods include steps for calculating a first similarity score and a second similarity score using the gene expression levels. In some embodiments, the classifying is based on the first and second similarity scores. In some embodiments, the first similarity score indicates whether the differentiation state of the population of cells is more similar to a first differentiation state or a second differentiation state. In some embodiments, the second similarity score indicates whether the differentiation state of the population of cells is more similar to the second differentiation state or a third differentiation state.

The first, second, and third differentiation states can be in the same or different stem cell differentiation pathways. In some embodiments, the first, second, and third differentiation states are all in the same stem cell differentiation pathway. In some embodiments, the second differentiation state is an intermediate differentiation state relative to the first and third differentiation pathways. For instance, in some embodiments, the first differentiation state is earlier in the stem cell differentiation pathway than the second differentiation state, and the second differentiation state is earlier in the stem cell differentiation pathway than the third differentiation state. In other embodiments, the second and third differentiation states are in different stem cell differentiation pathways, and the first differentiation state is that of cells that can differentiate into either the second or third differentiation state.

The provided methods allow for the determination of cell identity, e.g., cell differentiation state, when a single or small number of features or characteristics, such as gene expression markers or functional properties, are unavailable (e.g., unknown) or cannot be practically used to determine cell identity, e.g., cell differentiation state. In some aspects, certain cell populations that are differentiated from pluripotent stem cells, including determined dopaminergic cells, may be cells in a stage of differentiation where the cells are not identifiable by one or a small number of features or characteristics. In some aspects, differentiating cells can enter differentiation states where no definitive biomarker can be used to determine the identity, e.g., differentiation state, of the cells. While pluripotent stem cells can be positively identified with definitive biomarkers, for instance the expression levels of specific genes, and differentiated cells can be positively identified based on functional markers, individual markers for the identification of cells at various transient stages throughout differentiation are unknown. Without such markers, there has been previous difficulty in characterizing, defining, and/or identifying pre-differentiated cells with particular cell phenotypes. In some aspects, the methods provided herein overcome the lack of a single or small number of features or characteristics (e.g., biomarkers) by examining groups of genes and expression levels thereof. Such an approach does not rely on knowledge of individual marker genes and instead uses a whole transcriptome approach in characterizing and identifying the differentiation state of cells.

Induced pluripotent stem cells (iPSCs) are considered useful as a cell therapy for at least their ability to be differentiated into specialized cell types. For example, iPSCs, like pluripotent stem cells, can be differentiated into specific cell types that can be used to replace diseased or damaged tissue. In some cases, the therapeutic treatment can include administering (e.g., injecting) to the subject differentiating cells that have not entered a final differentiation state. The inability to determine the identity of the differentiated cells throughout the differentiation process can lead to uncertainty about the success of the process. For example, the differentiation process may need to be run to completion in order to determine if the differentiation process was successful. Thus, without the ability to determine whether differentiating cells are progressing through the transient stages as needed, the differentiation process becomes time consuming and inefficient, and can hinder treatment of a subject, for example when a differentiation process fails. In some embodiments, the provided methods improve the differentiation process, for example, by allowing a determination of cell identity throughout the states of differentiation, which can be used to determine whether cells undergoing a differentiation process are differentiating appropriately and/or according to defined standards. As an example, if it is determined that the cells are not differentiating appropriately, the process can be terminated and optionally reinitiated with different iPSC clones from the subject.

For certain cell therapies using cells that are differentiated from pluripotent stem cells, it is advantageous to use cells that are at an intermediate stage of the differentiation process. The present methods and devices are, in some embodiments, useful for identifying cells that are at the intermediate stage that is most efficacious when used for cell therapy. As an example, neural cells obtained by differentiation from pluripotent stem cells may be more amenable to engraftment into the brain of a subject undergoing treatment when the neural cells are at an intermediate stage between earlier stages (e.g., that of precursor cells) and later stages (e.g., that of committed cells)

Also provided herein in some embodiments are computing devices, including for performing any of the provided methods. Also provided herein in some embodiments are compositions, articles of manufacture, and kits including populations of cells, including populations of cells classified by any of the provided methods as having a desired differentiation state. Also provided herein in some embodiments are methods for implanting into a subject a population of cells having a desired differentiation state, for instance as classified according to any of the provided methods.

All publications, including patent documents, scientific articles, and databases, referred to in this application are incorporated herein by reference in their entirety for all purposes to the same extent as if each individual publication were individually incorporated by reference. If a definition set forth herein is contrary to or otherwise inconsistent with a definition set forth in the patents, applications, published applications, and other publications that are herein incorporated by reference, the definition set forth herein prevails over the definition that is incorporated herein by reference.

The section headings used herein are for organizational purposes only and are not to be construed as limiting the subject matter described.

### I. DEFINITIONS

Unless defined otherwise, all terms of art, notations, and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known. Reference to “about” a value or parameter herein includes (and describes) embodiments that are directed to that value or parameter per se. For example, description referring to “about X” includes description of “X”.

As used herein, a statement that a cell or population of cells “express” or is “positive” for a particular marker refers to the detectable presence on or in the cell of a particular marker. When referring to a surface marker, the term refers to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for a cell known to be positive for the marker, and/or at a level substantially higher than that for a cell known to be negative for the marker. When referring to a marker in the cell, such as a transcriptional or translational product, the term refers to the presence of detectable transcriptional or translational product, for example, wherein the product is detected at a level substantially above the level detected carrying out the same procedure with a control under otherwise identical conditions and/or at a level substantially similar to that for a cell known to be positive for the marker, and/or at a level substantially higher than that for a cell known to be negative for the marker.

As used herein, a statement that a cell or population of cells “does not express” or is “negative” for a particular marker refers to the absence of substantial detectable presence on or in the cell of a particular marker. When referring to a surface marker, the term refers to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control under otherwise identical conditions, and/or at a level substantially lower than that for a cell known to be positive for the marker, and/or at a level substantially similar as compared to that for a cell known to be negative for the marker. When referring to a marker in the cell, such as a transcriptional or translational product, the term refers to the absence of detectable transcriptional or translational product, for example, wherein the product is not detected at a level substantially above the level detected carrying out the same procedure with a control under otherwise identical conditions, and/or at a level substantially lower than that for cell known to be positive for the marker, and/or at a level substantially similar as compared to that for a cell known to be negative for the marker.

The term “expression” or “expressed” as used herein in reference to a gene refers to the transcriptional and/or translational product of that gene. The level of expression of a DNA molecule in a cell may be determined on the basis of either the amount of corresponding mRNA that is present within the cell or the amount of protein encoded by that DNA produced by the cell (Sambrook et al., 1989, Molecular Cloning: A Laboratory Manual, 18.1-18.88).

As used herein, the term “stem cell” refers to a cell characterized by the ability of self-renewal through mitotic cell division and the potential to differentiate into any of multiple cell types. Among mammalian stem cells, embryonic and somatic stem cells can be distinguished. Embryonic stem cells reside in the blastocyst and give rise to embryonic tissues, whereas somatic stem cells reside in adult tissues for the purpose of tissue regeneration and repair.

“Self renewal” refers to the ability of a cell to divide and generate at least one daughter cell with the self-renewing characteristics of the parent cell. A second daughter cell may commit to a particular differentiation pathway. For example, a self-renewing hematopoietic stem cell can divide and form one daughter stem cell and another daughter cell committed to differentiation in the myeloid or lymphoid pathway.

As used herein, the term “progenitor cell” refers to a cell having the potential to differentiate into any of multiple cell types, but that has lost self-renewal capacity relative to stem cells. For instance, a progenitor cell upon cell division may produce two daughter cells that display a more differentiated (e.g., restricted) phenotype.

As used herein, the term “non-self-renewing cell” refers to a cell that undergoes cell division to produce daughter cells, neither of which have the differentiation potential of the parent cell type, for instance generating differentiated daughter cells.

As used herein, the term “adult stem cell” refers to an undifferentiated cell found in an individual after embryonic development. Adult stem cells multiply by cell division to replenish dying cells and regenerate damaged tissue. An adult stem cell has the ability to divide and create another cell like itself or to create a more differentiated cell. Even though adult stem cells are associated with the expression of pluripotency markers such as Rex1, Nanog, Oct4, or Sox2, they do not have the ability of pluripotent stem cells to differentiate into the cell types of all three germ layers.

As used herein, the term “pluripotent” or “pluripotency” refers to cells with the ability to give rise to progeny that can undergo differentiation, under appropriate conditions, into cell types that collectively exhibit characteristics associated with cell lineages from the three germ layers (endoderm, mesoderm, and ectoderm). Pluripotent stem cells can contribute to tissues of a prenatal, postnatal, or adult organism.

As used herein, the term “pluripotent stem cell characteristics” refer to characteristics of a cell that distinguish pluripotent stem cells from other cells. Expression or non-expression of certain combinations of molecular markers are examples of characteristics of pluripotent stem cells. More specifically, human pluripotent stem cells may express at least some, and optionally all, of the markers from the following non-limiting list: SSEA-3, SSEA-4, TRA-1-60, TRA-1-81, TRA-2-49/6E, ALP, Sox2, E-cadherin, UTF-1, Oct4, Lin28, Rex1, and Nanog. Cell morphologies associated with pluripotent stem cells are also pluripotent stem cell characteristics.

As used herein, the terms “induced pluripotent stem cell,” “iPS,” and “iPSC” refer to a pluripotent stem cell artificially derived (e.g., through man-made manipulation) from a non-pluripotent cell. A “non-pluripotent cell” can be a cell of lesser potency to self-renew and differentiate than a pluripotent stem cell. Cells of lesser potency can be adult stem cells, tissue specific precursor cells, or primary or secondary cells.

The term “specification” or “specified” as provided herein refers to the fate of a cell or tissue narrowed to a limited number of specific cell types. A specified cell can still change its specific fate until it reaches the determined state. A specified cell can be capable of differentiating autonomously (e.g., by itself) when placed in an environment that is neutral with respect to the developmental pathway, such as in a petri dish or test tube. At the stage of specification, cell commitment may still be capable of being altered. If a specified cell is transplanted to a population of differently specified cells, the fate of the transplant can be altered by its interactions with its new neighbors.

A “determined state” as used herein refers to a cell having only one cell type it can differentiate into. For example, determined dopaminergic cells cannot become other types of neurons, though they may not yet be dopaminergic neurons themselves and may or may not express definitive markers of dopaminergic neurons. A determined cell may also be capable of differentiating autonomously when placed into a region of an embryo that is unrelated to said cell. For example, an unrelated region for a determined dopaminergic cell is any organ or tissue other than the brain. A determined cell can also be capable of differentiating autonomously when placed into a cluster of differently specified cells in a petri dish.

The term “differentiated” or “committed” as used herein refers to a cell or cells that have acquired a cell type-specific function.

A “neuronal precursor cell” is a cell that has a tendency to differentiate into a neuronal or glial cell and does not have the pluripotent potential of a stem cell. A neuronal precursor is a cell that is committed to the neuronal or glial lineage and is characterized by expressing one or more marker genes that are specific for the neuronal or glial lineage. The terms “neural” and “neuronal” are used according to their common meaning in the art and can be used interchangeably herein throughout.

A “dopaminergic cell” or a “differentiated dopaminergic cell” as used herein refers to a cell capable of synthesizing the neurotransmitter dopamine. In some embodiments, the dopaminergic cell is an A9 dopaminergic cell. The term “A9 dopaminergic cell” refers to the most densely packed group of dopaminergic cells in the human brain, which are located in the pars compacta of the substantia nigra in the midbrain of healthy, adult humans.

The term “determined dopaminergic cell” as used herein refers to a cell that will differentiate into a dopaminergic neuron and cannot differentiate into a non-dopaminergic cell. A “determined dopaminergic cell” is a cell able to differentiate into a dopaminergic neuron independently of its environment. A determined dopaminergic cell may express Foxa2 or Nurrl. A determined dopaminergic cell may not express serotonin.

As used herein, the term “reprogramming” refers to the process of dedifferentiating a non-pluripotent cell into a cell exhibiting pluripotent stem cell characteristics.

As used herein, the term “cell culture” may refer to an in vitro population of cells residing outside of an organism. The cell culture can be established from primary cells isolated from a cell bank or animal, or secondary cells that are derived from one of these sources and immortalized for long-term in vitro cultures.

As used herein, the terms “culture,” “culturing,” “grow,” “growing,” “maintain,” “maintaining,” “expand,” “expanding,” etc., when referring to cell culture itself or the process of culturing can be used interchangeably to mean that a cell is maintained outside the body (e.g., ex vivo) under conditions suitable for survival. Cultured cells are allowed to survive, and culturing can result in cell growth, differentiation, or division.

As used herein, a composition refers to any mixture of two or more products, substances, or compounds, including cells. It may be a solution, a suspension, liquid, powder, a paste, aqueous, non-aqueous or any combination thereof.

The term “pharmaceutical composition” refers to a composition suitable for pharmaceutical use, such as in a mammalian subject (e.g., a human). A pharmaceutical composition typically comprises an effective amount of an active agent (e.g., cells) and a carrier, excipient, or diluent. The carrier, excipient, or diluent is typically a pharmaceutically acceptable carrier, excipient, or diluent, respectively.

A “pharmaceutically acceptable carrier” refers to an ingredient in a pharmaceutical formulation other than an active ingredient that is nontoxic to a subject. A pharmaceutically acceptable carrier includes, but is not limited to, a buffer, excipient, stabilizer, or preservative.

The term “package insert” is used to refer to instructions customarily included in commercial packages of therapeutic products that contain information about the indications, usage, dosage, administration, combination therapy, contraindications, and/or warnings concerning the use of such therapeutic products.

As used herein, a “subject” is a mammal, such as a human or other animal, and typically is human.

### II. METHODS FOR CLASSIFYING OR IDENTIFYING CELLS

Provided herein in some embodiments are methods for classifying the differentiation state of an in vitro population of cells. In some embodiments, the provided methods are for identifying an in vitro population of cells having a desired differentiation state. In some embodiments, the provided methods are for selecting an in vitro population of cells having a desired differentiation state.

Also provided herein in some embodiments are methods for predicting if an in vitro population of cells will exhibit neurite outgrowth following implantation in a brain region. In some embodiments, the provided methods are for identifying an in vitro population of cells that will exhibit neurite outgrowth following implantation in a brain region. In some embodiments, the provided methods are for selecting an in vitro population of cells that will exhibit neurite outgrowth following implantation in a brain region.

In some embodiments, the provided methods are computer-implemented methods. In some embodiments, the provided methods are performed by a computing device. In some embodiments, the provided methods are performed by any of the provided computing devices, e.g., any as described in Section III.

In some embodiments, the provided methods provide, inter alia, information regarding whether an in vitro population of cells (e.g., a population of neuronal cells) includes cells that are determined to differentiate into a specific functional cell type (e.g., includes determined dopaminergic cells) or whether the in vitro population of cells includes cells from earlier stages (e.g., pluripotent stem cells, neuronal precursor cells), later stages (e.g., committed dopaminergic cells), or other differentiated cell types. In some embodiments, the provided methods predict whether an in vitro population of cells will differentiate into a specific cell type (e.g., into dopaminergic cells). In some embodiments, the cells identified with the provided methods are determined to differentiate into a specific functional cell type (e.g., into dopaminergic cells). Whether a cell is determined to differentiate into a specific functional cell type (e.g., whether the cell is a determined dopaminergic cell) may further be demonstrated in vitro or in vivo by allowing the cell to fully differentiate. The provided methods also encompass identifying cells that are pluripotent stem cells, specified cells, differentiating neuron types other than determined dopaminergic cells, or other differentiated cell types.

In some embodiments, the provided methods include receiving as input a test dataset that includes characteristics of one or more test cells. Exemplary test cells are described in Section II-C. In some embodiments, the provided methods include receiving as input a test dataset that includes expression levels for genes expressed in one or more test cells. The gene expression levels can be assessed using any of the methods described in Section II-D.

In some embodiments, the provided methods including calculating a first similarity score and a second similarity score. In some embodiments, the first similarity score indicates whether the differentiation state of the test cells is more similar to a first differentiation state or to a second differentiation state. In some embodiments, the second similarity score indicates whether the differentiation state of the test cells is more similar to the second differentiation state or to a third differentiation state. Exemplary methods for calculating the first and second similarity scores are described in Section II-A. Exemplary first, second, and third differentiation states are described in Section II-C.

In some embodiments, the differentiation state of the one or more test cells is classified based on one or both of the first and second similarity scores. In some embodiments, the provided methods include classifying the differentiation state of the one or more test cells based on one or both of the first and second similarity scores.

In some embodiments, the differentiation state of the one or more test cells is classified based on the first and second similarity scores. In some embodiments, the provided methods include classifying the differentiation state of the one or more test cells based on the first and second similarity scores.

In some embodiments, the differentiation state of the one or more test cells is classified as being the second differentiation state if one or both of the first and second similarity scores indicate that the differentiation state of the one or more test cells is more similar to the second differentiation state. In some embodiments, the provided methods include classifying the differentiation state of the one or more test cells as being the second differentiation state if one or both of the first and second similarity scores indicate that the differentiation state of the one or more test cells is more similar to the second differentiation state.

In some embodiments, the classifying is based on one of the first similarity score and the second similarity score. In some embodiments, the classifying is based on the lower of the first similarity score and the second similarity score. In some embodiments, the classifying is based on the higher of the first similarity score and the second similarity score. In some embodiments, the classifying is based on the first similarity score. In some embodiments, the classifying is based on the second similarity score.

In some embodiments, the differentiation state of the one or more test cells is classified as being the second differentiation state if the first and second similarity scores indicate that the differentiation state of the one or more test cells is more similar to the second differentiation state. In some embodiments, the provided methods include classifying the differentiation state of the one or more test cells as being the second differentiation state if the first and second similarity scores indicate that the differentiation state of the one or more test cells is more similar to the second differentiation state.

In some embodiments, the differentiation state of the one or more test cells is classified as being the second differentiation state, and the in vitro population of cells is identified as having the desired differentiation state. In some embodiments, the differentiation state of the one or more test cells is classified as being the second differentiation state, and the provided methods include identifying the in vitro population of cells as having the desired differentiation state.

In some embodiments, the provided methods include selecting the in vitro population of cells having the desired differentiation state for use in treating a disease or condition in a subject. In some embodiments, the in vitro population of cells having the desired differentiation state is selected for implantation in a subject. In some embodiments, the provided methods include implanting the in vitro population of cells having the desired differentiation state in a subject, e.g., according to any of the methods described in Section VI.

In some embodiments, the provided methods also include calculating a correlation score using characteristics of the one or more test cells and a control dataset. In some embodiments, the classifying the differentiation state of the one or more test cells is based on the correlation score and one or both of the first similarity score and the second similarity score. In some embodiments, the classifying is based on the correlation score and one of the first similarity score and the second similarity score. In some embodiments, the classifying is based on the correlation score and the lower of the first similarity score and the second similarity score. In some embodiments, the classifying is based on the correlation score and the higher of the first similarity score and the second similarity score. In some embodiments, the classifying is based on the correlation score and the first similarity score. In some embodiments, the classifying is based on the correlation score and the second similarity score. Exemplary methods for calculating the correlation score are described in Section II-B.

In some embodiments, the provided methods also include calculating a correlation score using characteristics of the one or more test cells and a control dataset. In some embodiments, the classifying the differentiation state of the one or more test cells is based on the first similarity score, the second similarity score, and the correlation score. Exemplary methods for calculating the correlation score are described in Section II-B.

In some embodiments, the provided methods involve the use of trained machine learning models. In some embodiments, the first and second similarity scores are determined using a first and second machine learning model, respectively. In some embodiments, the first and second similarity scores are determined based on one or more outputs of the first and second machine learning model, respectively. Exemplary model types for the first and second machine learning model are described in Section II-A-4. In some embodiments, the first and second machine learning models are each trained using characteristics, e.g., gene expression levels, of a plurality of reference cell populations. Exemplary reference cell populations are described in Section II-C.

Also provided herein in some embodiments is a method for training a machine learning model that can be used for classifying the differentiation state of an in vitro population of cells.

In some embodiments, the method includes training a first and second machine learning model. In some embodiments, the first and second machine learning models are trained using gene expression levels. Exemplary genes included and/or selected for model training are described in Section II-A-3. Exemplary model types for the first and second machine learning model are described in Section II-A-4. The gene expression levels can be assessed according to any of the methods described in Section II-D.

In some embodiments, the provided methods include obtaining, for a plurality of reference cell populations, gene expression levels for one or more genes that are differentially expressed between cells at a first differentiation state and cells at a second differentiation state. In some embodiments, the method includes selecting genes that are differentially expressed between cells at a first differentiation state and cells at a second differentiation state. In some embodiments, the method includes obtaining expression levels of one or more of the selected genes for a plurality of reference cell populations. In some embodiments, the gene expression levels for the plurality of reference cell populations are applied as input to train the first machine learning model. In some embodiments, one or more outputs of the trained first machine learning model can be used to classify the differentiation state of one or more test cells. In some embodiments, one or more outputs of the trained first machine learning model can be used to calculate a first similarity score indicating whether the differentiation state of test cells is more similar to the first differentiation state or to the second differentiation state.

In some embodiments, the provided methods include obtaining, for a plurality of reference cell populations, gene expression levels for one or more genes that are differentially expressed between cells at the second differentiation state and cells at a third differentiation state. In some embodiments, the method includes selecting genes that are differentially expressed between cells at the second differentiation state and cells at a third differentiation state. In some embodiments, the method includes obtaining expression levels of one or more of the selected genes for a plurality of reference cell populations. In some embodiments, the gene expression levels for the plurality of reference cell populations are applied as input to train the second machine learning model. In some embodiments, one or more outputs of the trained second machine learning model can be used to classify the differentiation state of one or more test cells. In some embodiments, one or more outputs of the trained second machine learning model can be used to calculate a second similarity score indicating whether the differentiation state of test cells is more similar to the second differentiation state or to the third differentiation state. Exemplary reference cell populations and first, second, and third differentiation states are described in Section II-C.

In some embodiments, the method further includes obtaining, for a plurality of reference cell populations, gene expression levels for one or more genes that are expressed in cells at a control differentiation state. The control differentiation state may be the same as or different than one of the first, second, or third differentiation states. Exemplary control differentiation states are described in Section II-C. In some embodiments, the method further includes applying the gene expression levels for the one or more genes as input to train a control machine learning model. In some embodiments, one or more outputs of the trained control machine learning model can be used to classify the differentiation state of one or more test cells. In some embodiments, one or more outputs of the trained control machine learning model can be used to determine if the differentiation state of test cells is similar to the control differentiation state.

A. Similarity Scores

In some embodiments, the provided methods including calculating a first similarity score and a second similarity score. In some embodiments, the differentiation state of the one or more test cells are classified based on one or both of the first and second similarity scores. In some embodiments, the differentiation state of the one or more test cells is classified as being the second differentiation state if one or both of the first and second similarity scores indicate that the differentiation state of the one or more test cells is similar to the second differentiation state. In some embodiments, the provided methods include classifying the differentiation state of the one or more test cells as being the second differentiation state if one or both of the first and second similarity scores indicate that the differentiation state of the one or more test cells is similar to the second differentiation state.

In some embodiments, the classifying is based on one of the first similarity score and the second similarity score. In some embodiments, the classifying is based on the lower of the first similarity score and the second similarity score. In some embodiments, the classifying is based on the higher of the first similarity score and the second similarity score. In some embodiments, the classifying is based on the first similarity score. In some embodiments, the classifying is based on the second similarity score.

In some embodiments, the provided methods including calculating a first similarity score and a second similarity score. In some embodiments, the differentiation state of the one or more test cells are classified based on the first and second similarity scores. In some embodiments, the differentiation state of the one or more test cells is classified as being the second differentiation state if the first and second similarity scores indicate that the differentiation state of the one or more test cells is similar to the second differentiation state. In some embodiments, the provided methods include classifying the differentiation state of the one or more test cells as being the second differentiation state if the first and second similarity scores indicate that the differentiation state of the one or more test cells is similar to the second differentiation state.

In some embodiments, the first and second similarity scores are calculated using gene expression levels of the test dataset. In some embodiments, the gene expression levels of the test dataset are compared to gene expression levels included in a first and second reference dataset. In some embodiments, the first and second similarity scores are calculated using representations of gene expression levels included in a first and second reference dataset, respectively. In some embodiments, the representations are obtained by machine learning. In some embodiments, the first and second reference datasets include a first and second machine learning model, respectively, and the first and second similarity scores are calculated by applying the gene expression levels of the test dataset as input to the first and second machine learning models, respectively, and are based on one or more outputs of the first and second machine learning models, respectively.

In some embodiments, one or both of the first and second similarity scores are binary outputs (e.g., 0 or 1, or −1 or 1) indicating if the differentiation state of the one or more test cells is the second differentiation state. In some embodiments, one or both of the first and second similarity scores are non-binary outputs.

Depending on the type of non-binary output, first and second similarity scores above or below a predetermined threshold level may indicate that the differentiation state of the one or more test cells is the second differentiation state. The predetermined threshold level can be the same or different for the first and second similarity scores. Any suitable method for setting the predetermined threshold level can be used. For instance, in some embodiments, the predetermined threshold level for the first similarity score is set based on a plurality of first similarity scores calculated using gene expression levels of a plurality of reference cell populations used to obtain the representations of gene expression levels of the first reference dataset, e.g., used to train the first machine learning model of the first reference dataset. In some embodiments, the predetermined threshold level is set at a value that separates the first similarity scores of reference cell populations that include cells of the first differentiation state and the first similarity scores of reference cell populations that include cells of the second differentiation state with an accuracy metric, e.g., accuracy, recall, precision, or F1 score, of at least 0.5, 0.6, 0.7, 0.8, 0.85, 0.9, 0.91, 0.92, 0.93, 0.94, 0.95, 0.96, 0.97, 0.98, or 0.99. Similarly, in some embodiments, the predetermined threshold level for the second similarity score is set based on a plurality of second similarity scores calculated using gene expression levels of the plurality of reference cell populations used to obtain the representations of gene expression levels of the second reference dataset, e.g., used to train the second machine learning model of the second reference dataset. In some embodiments, the predetermined threshold level is set at a value that separates the second similarity scores of reference cell populations that include cells of the second differentiation state and the second similarity scores of reference cell populations that include cells of the third differentiation state with an accuracy metric, e.g., accuracy, recall, precision, or F1 score, of at least 0.5, 0.6, 0.7, 0.8, 0.85, 0.9, 0.91, 0.92, 0.93, 0.94, 0.95, 0.96, 0.97, 0.98, or 0.99

In some embodiments, one or both of the first and second similarity scores are probabilities of the differentiation state of the one or more test cells being the second differentiation state. In some embodiments, a probability exceeding a predetermined probability threshold level indicates that the differentiation state of the one or more test cells is the second differentiation state. The predetermined probability threshold level can be the same or different for the first and second similarity scores. In some embodiments, the predetermined probability threshold level is, is about, is greater than, or is greater than about 0.5. In some embodiments, the predetermined probability threshold level is, is about, is greater than, or is greater than about 0.55. In some embodiments, the predetermined probability threshold level is, is about, is greater than, or is greater than about 0.6. In some embodiments, the predetermined probability threshold level is, is about, is greater than, or is greater than about 0.65. In some embodiments, the predetermined probability threshold level is, is about, is greater than, or is greater than about 0.7. In some embodiments, the predetermined probability threshold level is, is about, is greater than, or is greater than about 0.75. In some embodiments, the predetermined probability threshold level is, is about, is greater than, or is greater than about 0.8. In some embodiments, the predetermined probability threshold level is, is about, is greater than, or is greater than about 0.85. In some embodiments, the predetermined probability threshold level is, is about, is greater than, or is greater than about 0.9. In some embodiments, the predetermined probability threshold level is, is about, is greater than, or is greater than about 0.91. In some embodiments, the predetermined probability threshold level is, is about, is greater than, or is greater than about 0.92. In some embodiments, the predetermined probability threshold level is, is about, is greater than, or is greater than about 0.93. In some embodiments, the predetermined probability threshold level is, is about, is greater than, or is greater than about 0.94. In some embodiments, the predetermined probability threshold level is, is about, is greater than, or is greater than about 0.95. In some embodiments, the predetermined probability threshold level is, is about, is greater than, or is greater than about 0.96. In some embodiments, the predetermined probability threshold level is, is about, is greater than, or is greater than about 0.97. In some embodiments, the predetermined probability threshold level is, is about, is greater than, or is greater than about 0.98. In some embodiments, the predetermined probability threshold level is, is about, is greater than, or is greater than about 0.99.

In some embodiments, one or both of the first and second similarity scores are each compared to a predetermined threshold level. In some embodiments, one of the first and second similarity scores is compared to a predetermined threshold level. In some embodiments, the similarity score that is compared to its predetermined threshold level is based on which similarity score is closest to its predetermined threshold level. In some aspects, the similarity score that is compared to its predetermined threshold level is selected such that if the selected similarity score indicates that the differentiation state of test cells is more similar to the second differentiation state, it is expected that the other similarity score would also indicate that the differentiation state of test cells is more similar to the second differentiation state.

1. First Similarity Score

In some aspects, the provided methods involve calculating a first similarity score indicating whether the differentiation state of test cells is more similar to a first differentiation state or to a second differentiation state. In some embodiments, the first similarity score is calculated using a first reference dataset that includes gene expression levels for one or more genes differentially expressed between cells at the first differentiation state and cells at the second differentiation state. In some embodiments, the first similarity score is calculated using a first reference dataset that includes a representation of gene expression levels for one or more genes differentially expressed between cells at the first differentiation state and cells at the second differentiation state.

In some embodiments, the first reference dataset includes gene expression levels for one or more genes that are differentially expressed between cells at a first differentiation state and cells at a second differentiation state. In some embodiments, the gene expression levels are normalized gene expression levels. In some embodiments, the first similarity score is obtained by comparing the gene expression levels of the first reference dataset to the gene expression levels of the test dataset.

In some embodiments, the first reference dataset includes a representation of gene expression levels for one or more genes that are differentially expressed between cells at a first differentiation state and cells at a second differentiation state. In some embodiments, the representation of gene expression levels is obtained by machine learning. In some embodiments, the representation of gene expression levels is obtained by training a first machine learning model using gene expression levels of the one or more genes.

In some embodiments, the first similarity score is calculated using a first reference dataset that includes a first machine learning model that is trained using gene expression levels of one or more genes that are differentially expressed between cells at a first differentiation state and cells at a second differentiation state. In some embodiments, the first similarity score is calculated by providing gene expression levels of the test dataset as input to the first machine learning model or to a process that includes the first machine learning model. For instance, in some embodiments, the gene expression levels of the test dataset are normalized or transformed prior to being provided as input to the first machine learning model. In some embodiments, the first similarity score is an output of the first machine learning model. In some embodiments, the first similarity score is calculated using one or more outputs of the first machine learning model.

In some embodiments, the representation of gene expression levels of the first reference dataset is obtained using gene expression levels of a plurality of reference cell populations. In some embodiments, the first machine learning model is trained using gene expression levels of a plurality of reference cell populations. In some embodiments, the plurality of reference cell populations includes at least one reference cell population that includes cells of the first differentiation state and at least one reference cell population that includes cells of the second differentiation state. In some embodiments, the plurality of reference cell populations includes a plurality of reference cell populations that include cells of the first differentiation state and a plurality of reference cell populations that include cells of the second differentiation state. In some embodiments, the reference cell populations, e.g., those that include cells of the first or second differentiation state, are any as described in Section II-C.

2. Second Similarity Score

In some aspects, the provided methods involve calculating a second similarity score indicating whether the differentiation state of test cells is more similar to the second differentiation state or to a third differentiation state. In some embodiments, the second similarity score is calculated using a second reference dataset that includes gene expression levels for one or more genes differentially expressed between cells at the second differentiation state and cells at the third differentiation state. In some embodiments, the second similarity score is calculated using a second reference dataset that includes a representation of gene expression levels for one or more genes differentially expressed between cells at the second differentiation state and cells at the third differentiation state.

In some embodiments, the second reference dataset includes gene expression levels for one or more genes that are differentially expressed between cells at the second differentiation state and cells at a third differentiation state. In some embodiments, the gene expression levels are normalized gene expression levels. In some embodiments, the second similarity score is obtained by comparing the gene expression levels of the second reference dataset to the gene expression levels of the test dataset.

In some embodiments, the second reference dataset includes a representation of gene expression levels for one or more genes that are differentially expressed between cells at the second differentiation state and cells at a third differentiation state. In some embodiments, the representation of gene expression levels is obtained by machine learning. In some embodiments, the representation of gene expression levels is obtained by training a second machine learning model using gene expression levels of the one or more genes.

In some embodiments, the second similarity score is calculated using a second reference dataset that includes a second machine learning model that is trained using gene expression levels of one or more genes that are differentially expressed between cells at the second differentiation state and cells at a third differentiation state. In some embodiments, the second similarity score is calculated by providing gene expression levels of the test dataset as input to the second machine learning model or to a process that includes the second machine learning model. For instance, in some embodiments, the gene expression levels of the test dataset are normalized or transformed prior to being provided as input to the second machine learning model. In some embodiments, the second similarity score is an output of the second machine learning model. In some embodiments, the second similarity score is calculated using one or more outputs of the second machine learning model.

In some embodiments, the representation of gene expression levels of the second reference dataset is obtained using gene expression levels of a plurality of reference cell populations. In some embodiments, the second machine learning model is trained using gene expression levels of a plurality of reference cell populations. In some embodiments, the plurality of reference cell populations includes at least one reference cell population that includes cells of the second differentiation state and at least one reference cell population that includes cells of the third differentiation state. In some embodiments, the plurality of reference cell populations includes a plurality of reference cell populations that include cells of the second differentiation state and a plurality of reference cell populations that include cells of the third differentiation state. In some embodiments, the reference cell populations, e.g., those that include cells of the second or third differentiation state, are any as described in Section II-C.

3. Exemplary Genes

The one or more genes of the first and/or second reference datasets, e.g., the one or more genes used to train the first and/or second machine learning models, including in any of the provided methods involving training a first and/or second machine learning model, can be selected based on any suitable criteria. This criteria can include that the one or more genes are expressed above a minimum threshold level in the relevant cell populations, e.g., in reference cell populations comprising cells of the first, second, and/or third differentiation state, or in any combination of these reference cell populations. This criteria can also include that the one or more genes be differentially expressed between relevant cell populations (e.g., between cells of the first and second differentiation state, or between cells of the second and third differentiation state), for instance differentially expressed by a threshold fold-change level, with a certain statistical significance, or such that each of the one or more genes is individually predictive of differentiation state.

In some embodiments, the one or more genes of the first reference dataset or selected to train the first machine learning model include genes that increase in expression level from the first differentiation state to the second differentiation state. In some embodiments, the one or more genes of the first reference dataset or selected to train the first machine learning model include genes that decrease in expression level from the first differentiation state to the second differentiation state. In some embodiments, the one or more genes of the first reference dataset or selected to train the first machine learning model include genes that increase in expression level from the first differentiation state to the second differentiation state and genes that decrease in expression level from the first differentiation state to the second differentiation state.

In some embodiments, the one or more genes of the second reference dataset or selected to train the second machine learning model include genes that increase in expression level from the second differentiation state to the third differentiation state. In some embodiments, the one or more genes of the second reference dataset or selected to train the second machine learning model include genes that decrease in expression level from the second differentiation state to the third differentiation state. In some embodiments, the one or more genes of the second reference dataset or selected to train the second machine learning model include genes that increase in expression level from the second differentiation state to the third differentiation state and genes that decrease in expression level from the second differentiation state to the third differentiation state.

In some embodiments, the one or more genes of the first reference dataset or selected to train the first machine learning model are the same as the one or more genes of the second reference dataset or selected to train the second machine learning model. In some embodiments, the one or more genes of the first reference dataset or selected to train the first machine learning model are different from the one or more genes of the second reference dataset or selected to train the second machine learning model. In some embodiments, some of the one or more genes of the first reference dataset or selected to train the first machine learning model are included in the one or more genes of the second reference dataset or selected to train the second machine learning model. In some embodiments, none of the one or more genes of the first reference dataset or selected to train the first machine learning model are included in the one or more genes of the second reference dataset or selected to train the second machine learning model.

In some embodiments, the one or more genes of the first and/or second reference dataset or selected to train the first and/or second machine learning model include a plurality of genes. In some embodiments, the plurality of genes includes, includes about, includes greater than, or includes greater than about 2 genes. In some embodiments, the plurality of genes includes, includes about, includes greater than, or includes greater than about 3 genes. In some embodiments, the plurality of genes includes, includes about, includes greater than, or includes greater than about 4 genes. In some embodiments, the plurality of genes includes, includes about, includes greater than, or includes greater than about 5 genes. In some embodiments, the plurality of genes includes, includes about, includes greater than, or includes greater than about 6 genes. In some embodiments, the plurality of genes includes, includes about, includes greater than, or includes greater than about 7 genes. In some embodiments, the plurality of genes includes, includes about, includes greater than, or includes greater than about 8 genes. In some embodiments, the plurality of genes includes, includes about, includes greater than, or includes greater than about 9 genes. In some embodiments, the plurality of genes includes, includes about, includes greater than, or includes greater than about 10 genes. In some embodiments, the plurality of genes includes, includes about, includes greater than, or includes greater than about 12 genes. In some embodiments, the plurality of genes includes, includes about, includes greater than, or includes greater than about 14 genes. In some embodiments, the plurality of genes includes, includes about, includes greater than, or includes greater than about 16 genes. In some embodiments, the plurality of genes includes, includes about, includes greater than, or includes greater than about 18 genes. In some embodiments, the plurality of genes includes, includes about, includes greater than, or includes greater than about 20 genes. In some embodiments, the plurality of genes includes, includes about, includes greater than, or includes greater than about 25 genes. In some embodiments, the plurality of genes includes, includes about, includes greater than, or includes greater than about 30 genes. In some embodiments, the plurality of genes includes, includes about, includes greater than, or includes greater than about 35 genes. In some embodiments, the plurality of genes includes, includes about, includes greater than, or includes greater than about 40 genes. In some embodiments, the plurality of genes includes, includes about, includes greater than, or includes greater than about 45 genes. In some embodiments, the plurality of genes includes, includes about, includes greater than, or includes greater than about 55 genes. In some embodiments, the plurality of genes includes, includes about, includes greater than, or includes greater than about 60 genes. In some embodiments, the plurality of genes includes, includes about, includes greater than, or includes greater than about 62 genes. In some embodiments, the plurality of genes includes, includes about, includes greater than, or includes greater than about 64 genes. In some embodiments, the plurality of genes includes, includes about, includes greater than, or includes greater than about 66 genes. In some embodiments, the plurality of genes includes, includes about, includes greater than, or includes greater than about 68 genes. In some embodiments, the plurality of genes includes, includes about, includes greater than, or includes greater than about 70 genes. In some embodiments, the plurality of genes includes, includes about, includes greater than, or includes greater than about 80 genes. In some embodiments, the plurality of genes includes, includes about, includes greater than, or includes greater than about 90 genes. In some embodiments, the plurality of genes includes, includes about, includes greater than, or includes greater than about 100 genes. In some embodiments, the plurality of genes includes, includes about, includes greater than, or includes greater than about 110 genes. In some embodiments, the plurality of genes includes, includes about, includes greater than, or includes greater than about 120 genes. In some embodiments, the plurality of genes includes, includes about, includes greater than, or includes greater than about 130 genes. In some embodiments, the plurality of genes includes, includes about, includes greater than, or includes greater than about 140 genes. In some embodiments, the plurality of genes includes, includes about, includes greater than, or includes greater than about 150 genes. In some embodiments, the plurality of genes includes, includes about, includes greater than, or includes greater than about 160 genes. In some embodiments, the plurality of genes includes, includes about, includes greater than, or includes greater than about 170 genes. In some embodiments, the plurality of genes includes, includes about, includes greater than, or includes greater than about 180 genes. In some embodiments, the plurality of genes includes, includes about, includes greater than, or includes greater than about 190 genes. In some embodiments, the plurality of genes includes, includes about, includes greater than, or includes greater than about 200 genes. In some embodiments, the plurality of genes includes, includes about, includes greater than, or includes greater than about 250 genes. In some embodiments, the plurality of genes includes, includes about, includes greater than, or includes greater than about 300 genes. In some embodiments, the plurality of genes includes, includes about, includes greater than, or includes greater than about 350 genes. In some embodiments, the plurality of genes includes, includes about, includes greater than, or includes greater than about 400 genes. In some embodiments, the plurality of genes includes, includes about, includes greater than, or includes greater than about 450 genes. In some embodiments, the plurality of genes includes, includes about, includes greater than, or includes greater than about 500 genes.

In some embodiments, the one or more genes of the first and/or second reference dataset include genes having a minimum expression level in cells of the first, second, and/or third differentiation state. In some embodiments, the one or more genes selected for training the first and/or second machine learning models are selected for having a minimum expression level in cells of the first, second, and/or third differentiation state. In some embodiments, the one or more genes include genes with read counts, e.g., counts per million mapped reads (CPM) or log₂ CPM, that are at least 1, 2, 3, 4, 5, 6, 7, 8, 9, 10, 12, 14, 16, 18, or 20.

In some embodiments, the one or more genes of the first and/or second reference dataset are genes that are differentially expressed with a certain statistical significance. In some embodiments, the one or more genes selected for training the first and/or second machine learning model are selected for being differentially expressed with a certain statistical significance. In some embodiments, the one or more genes are genes that are differentially expressed with an associated p-value of less than 0.05. In some embodiments, the one or more genes are genes that are differentially expressed with an associated p-value of less than 0.01. In some embodiments, the one or more genes are genes that are differentially expressed with an associated p-value of less than 0.001. In some embodiments, the one or more genes are genes that are differentially expressed with an associated p-value of less than 0.0001. In some embodiments, the p-value is an adjusted p-value. In some embodiments, the p-value is adjusted for multiple comparisons. Any suitable multiple comparison procedures can be used. In some embodiments, the p-value is a Bonferroni corrected p-value. In some embodiments, the p-value is a false discovery rate (FDR)-adjusted p-value. In some embodiments, the p-value is a Holm-Bonferroni corrected p-value.

In some embodiments, the one or more genes of the first reference dataset or selected to train the first machine learning model are selected from the genes listed in Table E1. In some embodiments, the one or more genes include 10 or more genes selected from the genes listed in Table E1. In some embodiments, the one or more genes include 20 or more genes selected from the genes listed in Table E1. In some embodiments, the one or more genes include 30 or more genes selected from the genes listed in Table E1. In some embodiments, the one or more genes include 40 or more genes selected from the genes listed in Table E1. In some embodiments, the one or more genes include 50 or more genes selected from the genes listed in Table E1. In some embodiments, the one or more genes include 60 or more genes selected from the genes listed in Table E1. In some embodiments, the one or more genes include 70 or more genes selected from the genes listed in Table E1. In some embodiments, the one or more genes include 80 or more genes selected from the genes listed in Table E1. In some embodiments, the one or more genes include 90 or more genes selected from the genes listed in Table E1. In some embodiments, the one or more genes include 100 or more genes selected from the genes listed in Table E1. In some embodiments, the one or more genes include 200 or more genes selected from the genes listed in Table E1. In some embodiments, the one or more genes include 300 or more genes selected from the genes listed in Table E1. In some embodiments, the one or more genes include 400 or more genes selected from the genes listed in Table E1. In some embodiments, the one or more genes include 500 or more genes selected from the genes listed in Table E1.

In some embodiments, the one or more genes of the second reference dataset or selected to train the second machine learning model are selected from the genes listed in Table E2. In some embodiments, the one or more genes include 10 or more genes selected from the genes listed in Table E2. In some embodiments, the one or more genes include 20 or more genes selected from the genes listed in Table E2. In some embodiments, the one or more genes include 30 or more genes selected from the genes listed in Table E2. In some embodiments, the one or more genes include 40 or more genes selected from the genes listed in Table E2. In some embodiments, the one or more genes include 50 or more genes selected from the genes listed in Table E2. In some embodiments, the one or more genes include 60 or more genes selected from the genes listed in Table E2. In some embodiments, the one or more genes include 70 or more genes selected from the genes listed in Table E2. In some embodiments, the one or more genes include 80 or more genes selected from the genes listed in Table E2. In some embodiments, the one or more genes include 90 or more genes selected from the genes listed in Table E2. In some embodiments, the one or more genes include 100 or more genes selected from the genes listed in Table E2. In some embodiments, the one or more genes include 200 or more genes selected from the genes listed in Table E2. In some embodiments, the one or more genes include 300 or more genes selected from the genes listed in Table E2. In some embodiments, the one or more genes include 400 or more genes selected from the genes listed in Table E2. In some embodiments, the one or more genes include 500 or more genes selected from the genes listed in Table E2.

In some embodiments, the one or more genes of the first and/or second reference dataset are genes that are differentially expressed by at least a certain amount. In some embodiments, the one or more genes selected to train the first and/or second machine learning model are selected for being differentially expressed by at least a certain amount. In some embodiments, the one or more genes are genes that exhibit at least a threshold fold increase or decrease in gene expression levels. In some embodiments, the one or more genes are genes that exhibit at least a threshold fold increase or decrease in gene expression levels and with a certain statistical significance, e.g., with any of the associated p-values described herein. In some embodiments, the one or more genes are genes that exhibit at least a 1-fold increase or decrease in gene expression levels. In some embodiments, the one or more genes are genes that exhibit at least a 2-fold increase or decrease in gene expression levels. In some embodiments, the one or more genes are genes that exhibit at least a 3-fold increase or decrease in gene expression levels. In some embodiments, the one or more genes are genes that exhibit at least a 4-fold increase or decrease in gene expression levels. In some embodiments, the one or more genes are genes that exhibit at least a 5-fold increase or decrease in gene expression levels. In some embodiments, the one or more genes are genes that exhibit at least a 6-fold increase or decrease in gene expression levels. In some embodiments, the one or more genes are genes that exhibit at least a 7-fold increase or decrease in gene expression levels. In some embodiments, the one or more genes are genes that exhibit at least a 8-fold increase or decrease in gene expression levels. In some embodiments, the one or more genes are genes that exhibit at least a 9-fold increase or decrease in gene expression levels. In some embodiments, the one or more genes are genes that exhibit at least a 10-fold increase or decrease in gene expression levels.

In some embodiments, the one or more genes of the first and/or second reference dataset are genes that are individually predictive of cells having one differentiation state or another, e.g., the first or second differentiation state for the first reference data set, and the second or third differentiation state for the second reference data set. In some embodiments, the one or more genes selected to train the first and/or second machine learning model are genes selected for being individually predictive of cells having one differentiation state or another, e.g., the first or second differentiation state for the first machine learning model, and the second or third differentiation state for the second machine learning model. The predictiveness of a gene can be assessed using any suitable accuracy metric, e.g., accuracy, recall, precision, or F1 score. In some embodiments, the predictiveness of a gene is its accuracy in classifying the differentiation state of cells based on a threshold expression level of the gene, wherein a cell or cells having expression level of the gene that is higher than the threshold are classified as having one differentiation state, and a cell or cells having expression level of the gene that is lower than the threshold are classified as having another differentiation state. In some embodiments, the accuracy is at least 80%. In some embodiments, the accuracy is at least 82%. In some embodiments, the accuracy is at least 84%. In some embodiments, the accuracy is at least 86%. In some embodiments, the accuracy is at least 88%. In some embodiments, the accuracy is at least 90%. In some embodiments, the accuracy is at least 92%. In some embodiments, the accuracy is at least 94%. In some embodiments, the accuracy is at least 96%. In some embodiments, the accuracy is at least 98%. In some embodiments, the accuracy is 100%.

4. Exemplary Machine Learning Models

Various machine learning models are suitable for use in classifying the differentiation state of cells based on gene expression levels and are within the scope of the disclosure. In some embodiments, the machine learning models of the first and second reference datasets are the same type of machine learning model, e.g., are both logistic regression models. In some embodiments, the machine learning models of the first and second reference datasets are different types of machine learning models, e.g., one logistic regression model and one support vector machine classifier. Similarly, the first and second machine learning models trained according to any of the provided methods can be the same or different types of machine learning models.

Any suitable method for training the machine learning models can be used, including any as described in Hastie et al., The Elements of Statistical Learning (2016); and Abu-Mostafa et al., Learning from Data (2012). Exemplary machine learning models are also described in Hastie et al., The Elements of Statistical Learning (2016); and Abu-Mostafa et al., Learning from Data (2012).

Further exemplary machine learning models are provided in this section. The machine learning models of the first and second reference datasets or the first and second machine learning models trained according to any of the provided methods can be any of the exemplary machine learning models described herein.

In some embodiments, the machine learning model includes a supervised machine learning model. In some embodiments, the machine learning model includes an unsupervised machine learning model. In some embodiments, the machine learning model includes a semi-supervised machine learning model. In some embodiments, the machine learning model includes a clustering method.

In some embodiments, the machine learning model includes a regression model. In some embodiments, the machine learning model includes a classification model. In some embodiments, the machine learning model includes a binary classification model. In some embodiments, the machine learning model includes a multiclass classification model.

In some embodiments, the machine learning model includes a linear model. In some embodiments, the machine learning model includes a non-linear model.

In some embodiments, the machine learning model includes a logistic regression model. In some embodiments, the machine learning model includes a linear regression model. In some embodiments, the machine learning model includes a multiple linear regression model. In some embodiments, the machine learning model includes a polynomial regression model. In some embodiments, the machine learning model includes a quantile regression model. In some embodiments, the machine learning model includes a principle components regression model. In some embodiments, the machine learning model includes a partial least regression model. In some embodiments, the machine learning model includes a support vector regression model. In some embodiments, the machine learning model includes an ordinal regression model. In some embodiments, the machine learning model includes a Poisson regression model. In some embodiments, the machine learning model includes a negative binomial regression model. In some embodiments, the machine learning model includes a quasi Poisson regression model. In some embodiments, the machine learning model includes a linear discriminant analysis (LDA) model. In some embodiments, the machine learning model includes a Naïve Bayes classifier. In some embodiments, the machine learning model includes a perceptron. In some embodiments, the machine learning model includes a support vector machine (SVM). In some embodiments, the machine learning model includes a quadratic classifier. In some embodiments, the machine learning model includes a decision tree. In some embodiments, the machine learning model includes a random forest. In some embodiments, the machine learning model includes a neural network.

In some embodiments, the machine learning model includes a connectivity-based clustering method. In some embodiments, the machine learning model includes hierarchical clustering. In some embodiments, the machine learning model includes a centroid-based clustering method. In some embodiments, the machine learning model includes k-means clustering. In some embodiments, the machine learning model includes a distribution-based clustering method. In some embodiments, the machine learning model includes Gaussian mixture modeling. In some embodiments, the machine learning model includes a density-based clustering method. In some embodiments, the machine learning model includes DBSCAN. In some embodiments, the machine learning model includes OPTICS. In some embodiments, the machine learning model includes a grid-based clustering method. In some embodiments, the machine learning model includes STING. In some embodiments, the machine learning model includes CLIQUE.

In some embodiments, the machine learning model includes factor analysis. In some embodiments, the machine learning model includes network component analysis. In some embodiments, the machine learning model includes linear discriminant analysis. In some embodiments, the machine learning model includes independent component analysis (ICA). In some embodiments, the machine learning model includes principal component analysis (PCA). In some embodiments, the machine learning model includes sparse PCA. In some embodiments, the machine learning model includes robust PCA.

In some embodiments, the machine learning model includes non-negative matrix factorization (NMF). In some embodiments, the machine learning model includes conventional NMF. In some embodiments, the machine learning model includes discriminant NMF. In some embodiments, the machine learning model includes regularized NMF. In some embodiments, the machine learning model includes graph regularized NMF. In some embodiments, the machine learning model includes bootstrapping sparse NMF.

In some embodiments, the machine learning model includes kernel PCA. In some embodiments, the machine learning model includes generalized discriminant analysis (GDA). In some embodiments, the machine learning model includes an autoencoder. In some embodiments, the machine learning model includes T-distributed Stochastic Neighbor Embedding (t-SNE). In some embodiments, the machine learning model includes a manifold learning technique. In some embodiments, the machine learning model includes Isomap. In some embodiments, the machine learning model includes locally linear embedding (LLE). In some embodiments, the machine learning model includes Hessian LLE. In some embodiments, the machine learning model includes Laplacian eigenmaps. In some embodiments, the machine learning model includes graph-based kernel PCA. In some embodiments, the machine learning model includes uniform manifold approximation and projection (UMAP).

In some embodiments, the machine learning model includes a penalized machine learning model. In some embodiments, the machine learning includes a penalized version of any of the foregoing models. A penalized machine learning model is one in which coefficient estimates are regularized or constrained towards zero. In some embodiments, the machine learning model includes a ridge regression model. In some embodiments, the machine learning model includes a lasso regression model. In some embodiments, the machine learning model includes an elastic net regression model.

In some embodiments, the machine learning model includes an ensemble model. In some embodiments, the ensemble model involves a boosting algorithm. In some embodiments, the ensemble model involves a bagging algorithm.

In some embodiments, the machine learning model includes an ensemble model that includes a plurality of any combination of any of the foregoing models.

B. Correlation Score

In some embodiments, the test dataset includes gene expression levels for one or more genes whose expression levels are included in a control dataset. In some embodiments, the test dataset includes gene expression levels for one or more genes having a representation of expression levels included in a control dataset.

In some embodiments, the provided methods further include calculating a correlation score. In some embodiments, the correlation score indicates the similarity of the gene expression levels in the test dataset to the gene expression levels in the control dataset. In some embodiments, the correlation score indicates the similarity of the gene expression levels in the test dataset to the representation of gene expression levels in the control dataset.

In some embodiments, the calculating of the correlation score includes calculating a degree of correlation between the gene expression levels or the representations thereof in the control dataset and the gene expression levels in the test dataset. Any suitable measure indicating degree of correlation can be used, including Pearson correlation coefficient, Spearman's rank correlation, and mutual information.

In some embodiments, the differentiation state of the one or more test cells is classified based on the correlation score and one or both of the first similarity score and the second similarity score. In some embodiments, the provided methods further include classifying the differentiation state of the one or more test cells based on the correlation score and one or both of the first similarity score and the second similarity score.

In some embodiments, the classifying is based on the correlation score and one of the first similarity score and the second similarity score. In some embodiments, the classifying is based on the correlation score and the lower of the first similarity score and the second similarity score. In some embodiments, the classifying is based on the correlation score and the higher of the first similarity score and the second similarity score. In some embodiments, the classifying is based on the correlation score and the first similarity score. In some embodiments, the classifying is based on the correlation score and the second similarity score.

In some embodiments, the differentiation state of the one or more test cells is not classified as being the desired differentiation state if the correlation score indicates dissimilarity between the gene expression levels in the test dataset and the gene expression levels or representations thereof in the control dataset. In some embodiments, the differentiation state of the one or more test cells is not classified as being the desired differentiation state if the degree of correlation does not exceed a predetermined cutoff value. In some embodiments, the differentiation state of the one or more test cells is not classified as being the desired differentiation state if the correlation score indicates that the correlation or explained variance between the gene expression levels or representations thereof of the control dataset and the gene expression levels of the test dataset is less than or less than about 0.5, 0.55, 0.6, 0.65, 0.7, 0.75, 0.8, 0.85, 0.9, or 0.95.

In some embodiments, the differentiation state of the one or more test cells is classified based on the first similarity score, the second similarity score, and the correlation score. In some embodiments, the provided methods further include classifying the differentiation state of the one or more test cells based on the first similarity score, the second similarity score, and the correlation score. In some embodiments, the differentiation state of the one or more test cells is not classified as being the desired differentiation state if the correlation score indicates dissimilarity between the gene expression levels in the test dataset and the gene expression levels or representations thereof in the control dataset. In some embodiments, the differentiation state of the one or more test cells is not classified as being the desired differentiation state if the degree of correlation does not exceed a predetermined cutoff value. In some embodiments, the differentiation state of the one or more test cells is not classified as being the desired differentiation state if the correlation score indicates that the correlation or explained variance between the gene expression levels or representations thereof of the control dataset and the gene expression levels of the test dataset is less than or less than about 0.5, 0.55, 0.6, 0.65, 0.7, 0.75, 0.8, 0.85, 0.9, or 0.95.

In some embodiments, the correlation score is calculated prior to, concurrent with, or subsequent to the calculating of the first and second similarity scores. In some embodiments, the correlation score is calculated prior to the calculating of the first and second similarity scores. In some embodiments, the provided method is terminated if the correlation score indicates dissimilarity between the gene expression levels in the test dataset and the gene expression levels or representations thereof in the control dataset. In some embodiments, the provided method is terminated if the degree of correlation does not exceed a predetermined cutoff value.

In some embodiments, the one or more genes of the control dataset include genes that are expressed in cells at a control differentiation state. In some embodiments, the control differentiation state is any of the differentiation states described in Section II-C. In some embodiments, the control differentiation state is the same as one of the first, second, and third differentiation states. In some embodiments, the control differentiation state is different from the first, second, and third differentiation states.

In some embodiments, the one or more genes of the control dataset include genes that are expressed in cells at any of a plurality of control differentiation states. In some embodiments, the one or more genes of the control dataset include genes that are expressed in cells at each of a plurality of control differentiation states. In some embodiments, each of the plurality of control differentiation states is independently selected from any of the differentiation states described in Section II-C. In some embodiments, the plurality of control differentiation states include the first, second, and third differentiation states.

In some embodiments, the gene expression levels or representations thereof in the control dataset are based on gene expression levels of a plurality of reference cell populations. In some embodiments, the plurality of reference cell populations include the reference cell populations whose gene expression levels were used to train the first and second machine learning models, or include reference cell populations similar to those used to train the first and second machine learning models, for instance those of the same cell type or from the same stem cell differentiation pathway. Thus, in some aspects, the calculation of the correlation score allows for the comparison of the test dataset to gene expression levels of cells across the first, second, and/or third differentiation state.

In some embodiments, the plurality of reference cell populations are different from, e.g., do not include, the reference cell populations whose gene expression levels were used to train the first and second machine learning models. For instance, in some embodiments, the plurality of reference cell populations include different cell types and/or differentiation states than the reference cell populations whose gene expression levels were used to train the first and second machine learning models. Thus, in some aspects, the calculation of the correlation score allows for the comparison of the test dataset to gene expression levels of cells other than cells of the first, second, and/or third differentiation state.

In some embodiments, the one or more genes of the control dataset include genes having at least a minimum expression level in cells of the control differentiation state. In some embodiments, the one or more genes of the control dataset include genes having at least a minimum expression level in cells of any of the plurality of control differentiation states. In some embodiments, the one or more genes of the control dataset include genes having at least a minimum expression level in cells of each of the plurality of control differentiation states. In some embodiments, the one or more genes of the control dataset are expressed at the at least minimum expression level on average across a plurality of cell populations of the control differentiation state or plurality of control differentiation states.

In some embodiments, the one or more genes of the control dataset include genes with expression levels exceeding a threshold value. In some embodiments, the one or more genes of the control dataset have been filtered to only include genes whose expression levels exceed a threshold value. In some embodiments, the one or more genes of the control dataset include genes whose expression levels exceed the threshold value on average across a plurality of cell populations of the control differentiation state or plurality of control differentiation states. In some embodiments, the threshold value is a threshold CPM value. In some embodiments, the threshold value is a threshold log₂ CPM value. In some embodiments, the threshold value is or is about 1, 2, 3, 4, 5, 6, 7, 8, 9, 10, 12, 14, 16, 18, or 20. In some embodiments, the threshold value is or is about 10 CPM. In some embodiments, the threshold value is or is about 10 log₂ CPM.

In some embodiments, the gene expression levels of the control dataset include a representation of gene expression levels for the one or more genes. In some embodiments, the gene expression levels of the control dataset include normalized gene expression levels. In some embodiments, the gene expression levels of the control dataset are normalized by CPM, e.g., are CPM expression levels. In some embodiments, the gene expression levels of the control dataset are log-transformed. In some embodiments, the gene expression levels of the control dataset are log₂-transformed.

In some embodiments, the gene expression levels or representations thereof of the control dataset include average gene expression levels of a plurality of reference cell populations. In some embodiments, the degree of correlation is calculated between the gene expression levels in the test dataset and the average gene expression levels included in the control dataset. In some embodiments, the average gene expression levels include a centroid of gene expression levels. In some embodiments, the degree of correlation is calculated between the gene expression levels in the test dataset and the centroid of gene expression levels in the control dataset.

In some embodiments, the gene expression levels of the control dataset further include a measure of dispersion of gene expression levels of the plurality of reference cell populations. Any suitable measure of dispersion can be used, including standard deviation, range, interquartile range, mean absolute difference, median absolute deviation, average absolute deviation, distance standard deviation, coefficient of variation (CV), quartile coefficient of dispersion, relative mean difference, entropy, variance, and variance-to-mean ratio. In some embodiments, the measure of dispersion is standard deviation. In some embodiments, the measure of dispersion is coefficient of variation (CV).

In some embodiments, the degree of correlation is a weighted correlation value. In some embodiments, the correlation value is weighted by the measure of dispersion. In some embodiments, the correlation value is weighted by the inverse of the measure of dispersion. In some embodiments, the degree of correlation is a 1/CV-weighted correlation value. In some embodiments, the degree of correlation is a 1/CV-weighted correlation value calculated between the gene expression levels in the test dataset and the centroid value of gene expression levels in the control dataset.

C. Cell Populations

In some embodiments, the provided methods involve classifying the differentiation state of test cells. In some embodiments, the differentiation state of the test cells is classified based on representations of gene expression levels, e.g., machine learning models, that are based on gene expression levels from a plurality of reference cell populations. In some embodiments, a machine learning model used in the provided methods is trained using gene expression levels from a plurality of reference cell populations. In some embodiments, the machine learning models are trained to classify the differentiation state of test cells using gene expression levels from the plurality of reference cell populations.

1. Reference Cell Populations

In some aspects, the plurality of reference cell populations include cells of known identity, for instance of known cell type and/or differentiation state. For example, in some embodiments, the plurality of reference cell populations used in training the first machine learning model includes cells known to have the first or second differentiation state. Similarly, in some embodiments, the plurality of reference cell populations used in training the second machine learning model includes cells known to have the second or third differentiation state. In some embodiments, information regarding the known identity of the plurality of reference cell populations is used in training the machine learning models or used in establishing criteria to determine if the first and second similarity scores indicate if the differentiation state of the test cells is more similar to the second differentiation state.

In some embodiments, the plurality of reference cell populations are from cultures of cells that are differentiated from pluripotent cells subjected to suitable differentiation conditions. The provided methods can be performed with reference cell populations produced according to any differentiation method. Exemplary differentiation methods are described in Section II-C.

In some embodiments, the plurality of reference cell populations include cells differentiated under conditions to become dopaminergic neurons. In some embodiments, the plurality of reference cell populations include cells differentiated according to any of the methods described in Section II-C.

In some embodiments, the pluripotent stem cells are induced pluripotent stem cells (iPSCs). In some embodiments, the iPSCs are generated from fibroblasts collected from healthy human subjects. In some embodiments, the iPSCs are generated from fibroblasts collected from human subjects with Parkinson's disease. Exemplary methods for iPSC generation are described in Section II-C.

In some embodiments, the cells of the reference cell populations include pluripotent stem cells. In some embodiments, the pluripotent stem cells are induced pluripotent stem cells (iPSCs). In some embodiments, the iPSCs are generated from fibroblasts collected from a healthy human subject. In some embodiments, the iPSCs are generated from fibroblasts collected from a human subject having Parkinson's disease. In some embodiments, the iPSCs are generated from fibroblasts collected from a human subject predisposed to developing Parkinson's disease. Exemplary methods for iPSC generation are described in Section II-C.

In some embodiments, the cells of the reference cell populations include cells differentiated under conditions to become a neuronal cell, such as a floor plate midbrain precursor cells, determined dopaminergic cells, or a dopaminergic neuron. In some embodiments, the cells of the reference cell populations include cells differentiated according to any of the methods described in Section II-C. In some embodiments, the cells of the reference cell populations include determined dopaminergic cells. In some embodiments, the cells of the reference cell populations include dopaminergic neurons, e.g., committed dopaminergic neurons. In some embodiments, the cells of the reference cell populations include cells derived from iPSCs, for example iPSCs as described above, that have been cultured under conditions to promote differentiation into dopaminergic neurons.

In some embodiments, cells of the reference cell populations include dopaminergic neurons expressing a marker of a midbrain dopaminergic neuron, such as expression of FOXA2 or tyrosine hydroxylase (TH). In some embodiments, cells of the reference cell populations include cells expressing TH (TH+). In some embodiments, cells of the reference cell populations include cells expressing FOXA2 (FOXA2+). In some embodiments, cells of the reference cell populations include cells expressing TH and FOXA2 (TH+FOXA2+).

In some embodiments, cells of the reference cell populations include cells determined to or capable of becoming dopaminergic neurons, i.e., are determined dopaminergic cells, as ascertained based on one or more characteristics that indicate the cells are capable of having functional activity of a dopaminergic neuron but may not yet express a marker of a dopaminergic neuron or may not express it at a high level. For example, the cells may exhibit lower levels of TH than a dopaminergic neuron, yet still exhibit one or more characteristics of a determined dopaminergic cell indicating the cells are capable of having functional activity of a dopaminergic neuron. In some embodiments, the one or more characteristics include activity to survive, engraft, and/or innervate other cells when administered in vivo, e.g., to an animal model. In some embodiments, cells of the reference cell populations include cells that are capable of innervating host tissue following transplantation into an animal or human subject. In some embodiments, cells of the reference cell populations include cells that exhibit neurite outgrowth following transplantation into an animal or human subject. In some embodiments, cells of the reference cell populations include cells that survive following transplantation into an animal or human subject. In some embodiments, cells of the reference cell populations include cells that engraft following transplantation into an animal or human subject.

In some embodiments, cells of the reference cell populations include cells with therapeutic effect to treat a neurodegenerative disease. In some embodiments, the cells when implanted ameliorate or reverse symptoms of a neurodegenerative disease. In some embodiments, the neurodegenerative disease is Parkinson's disease. In some embodiments, the cells when implanted in the substantia nigra of a subject, e.g., patient, in need thereof improve Parkinsonian symptoms.

In some embodiments, cells of the reference cell populations include cells screened for their therapeutic effect to treat a neurodegenerative disease, such as determined in an animal model of a neurodegenerative disease. In some embodiments, the neurodegenerative disease is Parkinson's disease. In some embodiments, the reference cells are screened using an animal model of Parkinson's disease. Any suitable animal model of Parkinson's disease can be used for screening. In some embodiments, the animal model is a lesion model wherein animals received unilateral stereotaxic injection of 6-hydroxydopamine (6-OHDA) into the substantia nigra. In some embodiments, the animal model is a lesion model wherein animals received unilateral stereotaxic injection of 6-OHDA into the medial forebrain bundle. In some embodiments, the cells are implanted into the substantia nigra of the animal model. In some embodiments, a behavioral assay is performed to screen for therapeutic effects of the implantation on the animal model. In some embodiments, the behavioral assay comprises monitoring amphetamine-induced circling behavior. In some embodiments, the cells are determined to reduce, decrease or reverse a Parkinsonian model brain lesion in this model. In some embodiments, the cells may include cells that do not reduce, decrease, or reverse a Parkinsonian model brain lesion in this model. The reference cell populations may include various cells that exhibit varied or different therapeutic effects to treat a neurodegenerative disease, such as in an animal model.

2. Test Cells

In some aspects, the test cells are cells of unknown identity, for instance unknown cell type and/or differentiation state. In some embodiments, the test cells are known to be or are suspected to be of a certain stem cell differentiation pathway, but are of unknown differentiation state within the pathway. In some aspects, the provided methods allow for determining the cell type and/or differentiation state of the test cells based on gene expression levels of the test cells. Based on this determination, the in vitro population of cells containing the test cells can be classified as having a certain cell type and/or differentiation state.

In some embodiments, the in vitro population of cells containing the test cells is from a culture of cells that are differentiated from pluripotent cells subjected to suitable differentiation conditions. The provided methods can be performed with test cells produced according to any differentiation method. Exemplary differentiation methods and in vitro populations of cells are described in Section II-C.

In some embodiments, the cells are stem-cell derived neuronal cells. In some embodiments, the test cells include cells differentiated under conditions to become dopaminergic neurons. In some embodiments, the test cells include cells differentiated according to any of the methods described in Section II-C.

In some embodiments, the pluripotent stem cells are induced pluripotent stem cells (iPSCs). In some embodiments, the iPSCs are generated from fibroblasts collected from healthy human subjects. In some embodiments, the iPSCs are generated from fibroblasts collected from human subjects with Parkinson's disease. Exemplary methods for iPSC generation are described in Section II-C.

In some embodiments, the test cells are from an in vitro population of cells that is or is suspected to be in a different differentiation pathway from the reference cell populations. In some embodiments, the test cells are from an in vitro population of cells that has or is suspected to have been produced using different differentiation methods than those used to produce the reference cell populations.

In some embodiments, the test cells are from an in vitro population of cells that is or is suspected to be in the same differentiation pathway as the reference cell populations. In some embodiments, the test cells are from an in vitro population of cells that has or is suspected to have been produced using the same differentiation methods as those used to produce the reference cell populations. In some embodiments, the test cells are from an in vitro population of cells that is or is suspected to be in the same differentiation pathway as the reference cell populations, but that has or is suspected to have been produced using different differentiation methods than those used to produce the reference cell populations.

3. Exemplary Differentiation Pathways, Methods, and States

In some embodiments, the first differentiation state is earlier in a stem cell differentiation pathway than the second differentiation state. In some embodiments, the first differentiation state is later in a stem cell differentiation pathway than the second differentiation state. In some embodiments, the first and second differentiation states are from different stem cell differentiation pathways. In some embodiments, the first differentiation state is in a cell differentiation pathway that is parallel to the cell differentiation pathway of the second differentiation state. In some embodiments, the cell differentiation pathways are those that diverge, for instance such that the first and second differentiation states are of different cell types.

In some embodiments, the second differentiation state is earlier in a stem cell differentiation pathway than the third differentiation state. In some embodiments, the second differentiation state is later in a stem cell differentiation pathway than the third differentiation state. In some embodiments, the second and third differentiation states are from different stem cell differentiation pathways. In some embodiments, the second differentiation state is in a cell differentiation pathway that is parallel to the cell differentiation pathway of the third differentiation state. In some embodiments, the cell differentiation pathways are those that diverge, for instance such that the second and third differentiation states are of different cell types.

In some embodiments, the first, second, and third differentiation states are all in the same stem cell differentiation pathway. In some embodiments, the first, second, and third differentiation states are in different stem cell differentiation pathways. In some embodiments, the first and second differentiation states are in one stem cell differentiation pathway, and the first and third differentiation states are in another stem cell differentiation pathway, for instance pathways in which a cell in the first differentiation state is a precursor cell that can differentiate into a cell in the second or third differentiation state. In some embodiments, the second and third differentiation states are of different cell types.

In some embodiments, the first, second, and third differentiation states are all in the same stem cell differentiation pathway. In some embodiments, the second differentiation state is an intermediate differentiation state between the first and third differentiation states.

Exemplary in vitro populations of cells and differentiation states are provided in this section. This section is organized based on particular stem cell differentiation pathways, but combinations of first, second, third, and control differentiation states spanning multiple cell types or pathways are also contemplated and disclosed herein. For instance, in some embodiments, the first, second, and third differentiation states are all of the same cell type (e.g., neuronal). In other embodiments, at least one of the first, second, and third differentiation states may be of a cell type that differs from the remaining differentiation states (e.g., the first differentiation state being that of a neuronal cell, and the second and third differentiation states being that of cardiac cells).

In some embodiments, the test cells are from an in vitro population of stem-cell derived cardiac muscle cells (see, e.g., Le and Chong, Cell Death Discovery 2: 16052 (2016)). In some embodiments, the stem-cell derived cardiac muscle cells express Nkx2.5 and/or Isl-1. Exemplary methods for differentiating stem-cell derived cardiac muscle cells in vitro are described in U.S. Pat. No. 9,234,176, US20170058263, Vandat et al., Scientific Reports 9: 16006 (2019), Laflamme et al. (2007) Nature Biotechnology 25:1015-24, and Wu et al. (2021) Biosci Rep 41(6):BSR20200833. In some embodiments, the first, second, third, and/or control differentiation states are that of cardiac muscle precursor cells or determined or committed cardiomyocytes, endothelial cells, vascular smooth muscle cells, or cardiac fibroblasts. In some embodiments, the first differentiation state is that of cardiac muscle precursor cells; the second differentiation state is that of determined cardiomyocytes, endothelial cells, vascular smooth muscle cells, or cardiac fibroblasts; and the third differentiation state is the committed differentiation state corresponding to the second differentiation state. In some embodiments, the second differentiation state is that of determined cardiomyocytes. In some embodiments, the third differentiation state is that of committed cardiomyocytes. In some embodiments, the cells identified as having the desired differentiation state, e.g., having the second differentiation state, can be used in the treatment of degenerative diseases, such as ischemic cardiomyopathy and conduction system diseases (such as sinus node dysfunction and atrial-ventricular block), or congenital heart diseases, such as atrial or ventricular septal defects.

In some embodiments, the test cells are from an in vitro population from a culture of cells differentiated from pluripotent cells that are subjected to a differentiation protocol for inducing the differentiation of PSCs, e.g., iPSCs, into cardiomyocytes, such as according to any of the methods described herein, e.g., as described in Laflamme et al. (2007) Nature Biotechnology 25:1015-24 or Wu et al. (2021) Biosci Rep 41(6):BSR20200833. In some embodiments, cells of the second differentiation state are in any of days 14-21 of the differentiation protocol. In some embodiments, cells of the first differentiation state are at day 13 or earlier, day 12 or earlier, day 11 or earlier, or day 10 or earlier of the differentiation protocol. In some embodiments, cells of the third differentiation state are at day 22 or later, day 30 or later, day 40 or later, day 50 or later, day 60 or later, or day 70 or later of the differentiation protocol. In some embodiments, cells of the second differentiation state are in any of days 14-21 of the differentiation protocol; and cells of the third differentiation state are day 22 or later, day 30 or later, day 40 or later, day 50 or later, day 60 or later, or day 70 or later of the differentiation protocol. In some embodiments, cells of the first differentiation state are at day 13 or earlier, day 12 or earlier, day 11 or earlier, or day 10 or earlier of the differentiation protocol; cells of the second differentiation state are in any of days 14-21 of the differentiation protocol; and cells of the third differentiation state are at day 22 or later, day 30 or later, day 40 or later, day 50 or later, day 60 or later, or day 70 or later of the differentiation protocol. In some embodiments, cells of the third differentiation state are in any of days 70-126 of the differentiation protocol.

In some embodiments, the test cells are from an in vitro population of stem-cell derived skeletal muscle cells (see, e.g., Relaix et al., Nature Communications 12: 692 (2021)). In some embodiments, the stem-cell derived skeletal muscle cells express PAX7 and/or PAX3. Exemplary methods for differentiating stem-cell derived skeletal muscle cells in vitro are described in WO2001011011 and U.S. Pat. No. 9,789,136. In some embodiments, the first, second, third, and/or control differentiation states are that of skeletal muscle precursor cells, committed skeletal muscle cells, or determined skeletal muscle cells. In some embodments, the first differentiation state is that of skeletal muscle precursor cells; the second differentiation state is that of determined skeletal muscle cells; and the third differentiation state is that of committed skeletal muscle cells. In some embodiments, the cells identified as having the desired differentiation state, e.g., having the second differentiation state, can be used in the treatment of muscular disorders, such as myopathies, e.g., polymyositis, dermatomyositis, Duchenne muscular dystrophy; fibrositis; myasthenia gravis; rhabdomyolysis; amyotrophic lateral sclerosis; or sarcopenia.

In some embodiments, the test cells are from an in vitro population of stem-cell derived smooth muscle cells. Exemplary methods for differentiating stem-cell derived smooth muscle cells in vitro are described in U.S. Pat. No. 7,531,355. In some embodiments, the first, second, third, and/or control differentiation states are that of smooth muscle precursor cells, committed smooth muscle cells, or determined smooth muscle cells. In some embodments, the first differentiation state is that of smooth muscle precursor cells; the second differentiation state is that of determined smooth muscle cells; and the third differentiation state is that of committed smooth muscle cells. In some embodiments, the cells identified as having the desired differentiation state, e.g., having the second differentiation state, can be used to reconstitute tissue containing leiomyogenic cells (such as the urinary tract, epithelial pathway or bladder) or to treat disorders that affect smooth muscle function, e.g., urinary incontinence, bladder disease, vascular disorders, intestinal disorders, vesicoureteral reflux, or other disorders of smooth muscle function.

In some embodiments, the test cells are from an in vitro population of stem-cell derived vascular endothelial cells. Exemplary methods for differentiating stem-cell derived vascular endothelial cells in vitro are described in U.S. Pat. Nos. 10,041,036, 10,563,175, 10,828,337, 10,767,161, 9,938,499, and 10,947,506. In some embodiments, the first, second, third, and/or control differentiation states are that of vascular endothelial precursor cells, committed vascular endothelial cells, or determined vascular endothelial cells. In some embodments, the first differentiation state is that of vascular endothelial precursor cells; the second differentiation state is that of determined vascular endothelial cells; and the third differentiation state is that of committed vascular endothelial cells.

In some embodiments, the test cells are from an in vitro population of stem-cell derived kidney tubule cells (see, e.g., Chambers and Wingert, World J Stem Cells 2016; 8(11): 367-375). Exemplary methods for differentiating stem-cell derived kidney tubule cells in vitro are described in Ribeiro et al., Stem Cells Int. 2020: 8894590. In some embodiments, the first, second, third, and/or control differentiation states are that of kidney tubule precursor cells or commited or determined podocytes, proximal 51 cells, proximal S2 cells, proximal S3 cells, proximal tubule cells, DTL type 1 cells, DTL type 2 cells, DTL type 3 cells, ascending thin limb cells, MTAL limb cells, CTAL cells, macula densa cells, distal convoluted tubule cells, CNT cells, PC (CCD) cells, PC (OMCD) cells, Type A IC cells, Type B IC cells, or IMCD cells. In some embodiments, the first differentiation state is that of kidney tubule precursor cells; the second differentiation state is that of determined podocytes, proximal S1 cells, proximal S2 cells, proximal S3 cells, proximal tubule cells, DTL type 1 cells, DTL type 2 cells, DTL type 3 cells, ascending thin limb cells, MTAL limb cells, CTAL cells, macula densa cells, distal convoluted tubule cells, CNT cells, PC (CCD) cells, PC (OMCD) cells, Type A IC cells, Type B IC cells, or IMCD cells; and the third differentiation state is the committed differentiation state corresponding to the second differentiation state. In some embodiments, the cells identified as having the desired differentiation state, e.g., having the second differentiation state, can be used in the treatment of acute kidney injury, chronic kidne disease, refractory systemic lupus erythematosus, or lupus nephritis or for kidney transplants (see, e.g., Wong, World J Stem Cells, 2021; 13(7):914-933).

In some embodiments, the test cells are from an in vitro population of stem-cell derived red blood cell cells. Exemplary methods for differentiating stem-cell derived red blood cell cells in vitro are described in U.S. Pat. No. 1,027,211. In some embodiments, the first, second, third, and/or control differentiation states are that of red blood cell precursor cells, committed red blood cells, or determined red blood cells. In some embodments, the first differentiation state is that of red blood cell precursor cells; the second differentiation state is that of determined red blood cells; and the third differentiation state is that of committed red blood cells. In some embodiments, the cells identified as having the desired differentiation state, e.g., having the second differentiation state, can be used to treat disorders characterized by a deficiency of red blood cells, for instance to treat subjects having an auto-immune disorder, immune deficiency, or any other disease or disorder that would benefit from a transfusion of blood.

In some embodiments, the test cells are from an in vitro population of stem-cell derived lung cells (see, e.g., Leeman et al., Curr Top Dev Biol 2014; 107:207-233). In some embodiments, the stem-cell derived lung cells express Nkx2.1. Exemplary methods for differentiating stem-cell derived lung cells in vitro are described in U.S. Ser. No. 11/214,769 and WO2015108893. In some embodiments, the first, second, third, and/or control differentiation states are that of lung precursor cells or committed or determined airway epithelial cells, for instance goblet, ciliated, Clara, neuroendocrine (neuroendocrine bodies), basal, intermediate (or parabasal), serous, brush, oncocyte, nonciliated columnar, metaplastic (e.g., squamous or Clara-mucous cells, bronchiolar metaplasia) cells; or alveolar cells, for instance type 1 or type 2 pneumocytes or cuboidal nonciliated cells. In some embodiments, the first differentiation state is that of lung precursor cells; the second differentiation state is that of determined airway epithelial cells, for instance determined goblet, ciliated, Clara, neuroendocrine (neuroendocrine bodies), basal, intermediate (or parabasal), serous, brush, oncocyte, nonciliated columnar, metaplastic (e.g., squamous or Clara-mucous cells, bronchiolar metaplasia) cells; or determined alveolar cells, for instance determined type 1 or type 2 pneumocytes or cuboidal nonciliated cells; and the third differentiation state is the committed differentiation state corresponding to the second differentiation state. In some embodiments, the cells identified as having the desired differentiation state, e.g., having the second differentiation state, can be used to treat respiratory disorders, for instance cystic fibrosis, respiratory distress syndrome, acute respiratory distress syndrome, pulmonary tuberculosis, cough, bronchial asthma, cough based on increased airway hyperreactivity (bronchitis, flu syndrome, asthma, obstructive pulmonary disease, and the like), flu syndrome, anti-cough, airway hyperreactivity, tuberculosis disease, asthma (airway inflammatory cell infiltration, increased airway hyperresponsiveness, bronchoconstriction, mucus hypersecretion), chronic obstructive pulmonary disease, emphysema, pulmonary fibrosis, idiopathic pulmonary fibrosis, cough, reversible airway obstruction, adult respiratory disease syndrome, pigeon fancier's disease, farmer's lung, bronchopulmonary dysplasia, airway disorder, emphysema, allergic bronchopulmonary aspergillosis, allergic bronchitis bronchiectasis, occupational asthma, reactive airway disease syndrome, intersitial lung disease, or parasitic lung disease.

In some embodiments, the test cells are from an in vitro population of stem-cell derived thyroid cells. In some embodiments, the stem-cell derived thyroid cells express Pax-8 and/or NKX2-1. Exemplary methods for differentiating stem-cell derived thyroid cells in vitro are described in Fierabracci, Journal of Endocrinology 213(1):1-13 (2012). In some embodiments, the first, second, third, and/or control differentiation states are that of thyroid precursor cells or committed or determined follicular cells or C cells. In some embodiments, the first differentiation state is that of thyroid precursor cells; the second differentiation state is that of determined follicular or C cells; and the third differentiation state is the committed differentiation state corresponding to the second differentiation state. In some embodiments, the cells identified as having the desired differentiation state, e.g., having the second differentiation state, can be used to treat thyroid disorders, for instance goitre, adenomas, hypothyroidism, or autoimmune diseases.

In some embodiments, the test cells are from an in vitro population of stem-cell derived pancreatic cells. In some embodiments, the stem-cell derived pancreatic cells express Pdx1. In some embodiments, the stem-cell derived pancreatic cells are endocrine cells. In some embodiments, the stem-cell derived pancreatic cells are exocrine cells. Exemplary methods for differentiating stem-cell derived pancreatic cells in vitro are described in U.S. Pat. No. 8,859,286, WO2011011300, WO2014105543, WO2013095953, U.S. Pat. No. 9,157,062, and Balboa et al. (2022) Nature Biotechnology 40:1042-55. In some embodiments, the first, second, third, and/or control differentiation states are that of pancreatic precursor cells or committed or determined exocrine cells (e.g., acinar or ductal cells) or endocrine cells (e.g., beta, alpha, delta, or PP cells). In some embodiments, the first differentiation state is that of pancreatic precursor cells; the second differentiation state is that of determined exocrine cells (e.g., acinar or ductal cells) or endocrine cells (e.g., beta, alpha, delta, or PP cells); and the third differentiation state is the committed differentiation state corresponding to the second differentiation state. In some embodiments, the second differentiation state is that of determined beta cells. In some embodiments, the third differentiation state is that of committed beta cells. In some embodiments, the cells identified as having the desired differentiation state, e.g., having the second differentiation state, can be used to treat pancreatic disorders, metabolic disorders, or diseases involving the improper production or use of insulin, such as Type 1 diabetes.

In some embodiments, the test cells are from an in vitro population from a culture of cells differentiated from pluripotent cells that are subjected to a differentiation protocol for inducing the differentiation of PSCs, e.g., iPSCs, into beta cells, such as according to any of the methods described herein, e.g., as described in Balboa et al. (2022) Nature Biotechnology 40:1042-55. In some embodiments, cells of the second differentiation state are in any of days 21-35 of the differentiation protocol. In some embodiments, cells of the first differentiation state are at day 20 or earlier, day 19 or earlier, day 18 or earlier, or day 17 or earlier of the differentiation protocol. In some embodiments, cells of the third differentiation state are at day 36 or later, day 38 or later, day 40 or later, day 42 or later, or day 44 or later of the differentiation protocol. In some embodiments, cells of the second differentiation state are in any of days 21-35 of the differentiation protocol; and cells of the third differentiation state are at day 36 or later, day 38 or later, day 40 or later, day 42 or later, or day 44 or later of the differentiation protocol. In some embodiments, cells of the first differentiation state are at day 20 or earlier, day 19 or earlier, day 18 or earlier, or day 17 or earlier of the differentiation protocol; cells of the second differentiation state are in any of days 21-35 of the differentiation protocol; and cells of the third differentiation state are at day 36 or later, day 38 or later, day 40 or later, day 42 or later, or day 44 or later of the differentiation protocol. In some embodiments, cells of the third differentiation state are in any of days 56-98 of the differentiation protocol.

In some embodiments, the test cells are from an in vitro population of stem-cell derived epidermal cells (see, e.g., Jackson et al., Stem Cell Research & Therapy 8: 155 (2017)). Exemplary methods for differentiating stem-cell derived epidermal cells in vitro are described in U.S. Pat. No. 9,404,122. In some embodiments, the first, second, third, and/or control differentiation states are that of epidermal precursor cells or committed or determined karatinocytes, melanocytes, or Langerhans cells. In some embodiments, the first differentiation state is that of epidermal precursor cells; the second differentiation state is that of determined karatinocytes, melanocytes, or Langerhans cells; and the third differentiation state is the committed differentiation state corresponding to the second differentiation state. In some embodiments, the cells identified as having the desired differentiation state, e.g., having the second differentiation state, can be used to treat skin injuries or disorders, such as burns, chronic wounds, or stable vitiligo.

In some embodiments, the test cells are from an in vitro population of stem-cell derived pigment cells. In some embodiments, the stem-cell derived pigment cells are retinal pigment cells. In some embodiments, the stem-cell derived pigment cells are melanocytes. Exemplary methods for differentiating stem-cell derived pigment cells in vitro are described in WO2005070011, WO2011149762, WO2014121077, WO2009051671, and WO2008129554. In some embodiments, the first, second, third, and/or control differentiation states are that of pigment precursor cells or committed or determined retinal pigment cells or melanocytes. In some embodiments, the first differentiation state is that of pigment precursor cells; the second differentiation state is that of determined retinal pigment cells or melanocytes; and the third differentiation state is the committed differentiation state corresponding to the second differentiation state. In some embodiments, the cells identified as having the desired differentiation state, e.g., having the second differentiation state, can be used to treat degenerative diseases such as retinal degenerative disease, e.g., macular degeneration.

a. Neuronal Cells

In some embodiments, the test cells are from an in vitro population of stem-cell derived neuronal cells. Exemplary methods for differentiating stem-cell derived neuronal cells in vitro are described in WO2014176606, U.S. Pat. No. 8,460,931, U.S. Ser. No. 10/273,453, WO2012095730, U.S. Pat. No. 9,309,495, US20190249140, US20180298326, WO2009148170, WO2021146349, WO2021216623, WO2021216622. WO2013104752, WO2010096496, WO2013067362, WO2016196661, WO2015143342, and US20160348070.

In some embodiments, the methods of differentiating stem-cell derived neuronal cells can be methods that differentiate pluripotent stem cells, e.g., iPSCs, into any neural cell type using any available or known method for inducing the differentiation of pluripotent stem cells, e.g., iPSCs. In some embodiments, the method induces differentiation of the pluripotent stem cells into floor plate midbrain precursor cells, determined dopaminergic cells, and/or dopaminergic neurons. Any available and known method for inducing differentiation of pluripotent stem cells into floor plate midbrain precursor cells, determined dopaminergic cells, and/or dopaminergic neurons can be used.

In some embodiments, the method induces differentiation of the pluripotent stem cells into glial cells. In some embodiments, the glial cells are selected from the group consisting of microglial cells, astrocytes, oligodendrocytes, and ependymocytes.

In some embodiments, the test cells are from an in vitro population of stem-cell derived microglial cells. In some embodiments, the method induces differentiation of the pluripotent stem cells into microglial cells or microglial-like cells. Any available and known method for inducing differentiation of the pluripotent stem cells into microglial cells or microglial-like cells can be used. Exemplary methods of inducing differentiation of pluripotent stem cells into microglial cells or microglial-like cells can be found in, e.g., McQuade et al. (2018) Molecular Neurodegeneration 13:67; Abud et al., Neuron (2017), Vol. 94: 278-293; Douvaras et al., Stem Cell Reports (2017), Vol. 8: 1516-1524; Muffat et al., Nature Medicine (2016), Vol. 22(11): 1358-1367; and Pandya et al., Nature Neuroscience (2017), Vol. 20(5): 753-759. In some embodiments, the first, second, third, and/or control differentiation states are that of iPSCs, hematopoietic progenitor cells, or microglial cells. In some embodments, the first differentiation state is that of iPSCs; the second differentiation state is that of hematopoietic progenitor cells; and the third differentiation state is that of microglial cells. In some embodiments, the cells identified as having the desired differentiation state, e.g., having the second differentiation state, can be used for the treatment of Parkinson's disease, a Parkinsonism, an age-related neurodegenerative disease, Alzheimer's disease, or frontotemporal dementia.

In some embodiments, the test cells are from an in vitro population from a culture of cells differentiated from pluripotent cells that are subjected to a differentiation protocol for inducing the differentiation of PSCs, e.g., iPSCs, into microglial cells, such as according to any of the methods described herein. In some embodiments, cells of the second differentiation state are in any of days 28-35 of the differentiation protocol. In some embodiments, cells of the first differentiation state are at day 27 or earlier, day 26 or earlier, day 25 or earlier, or day 24 or earlier of the differentiation protocol. In some embodiments, cells of the third differentiation state are at day 36 or later, day 37 or later, day 38 or later, day 39 or later, day 40 or later, day 41 or later, day 42 or later, or day 43 or later of the differentiation protocol. In some embodiments, cells of the second differentiation state are in any of days 28-35 of the differentiation protocol; and cells of the third differentiation state are at day 36 or later, day 37 or later, day 38 or later, day 39 or later, day 40 or later, day 41 or later, day 42 or later, or day 43 or later of the differentiation protocol. In some embodiments, cells of the first differentiation state are at day 27 or earlier, day 26 or earlier, day 25 or earlier, or day 24 or earlier of the differentiation protocol; cells of the second differentiation state are in any of days 28-35 of the differentiation protocol; and cells of the third differentiation state are at day 36 or later, day 37 or later, day 38 or later, day 39 or later, day 40 or later, day 41 or later, day 42 or later, or day 43 or later of the differentiation protocol. In some embodiments, cells of the third differentiation state are in any of days 49-63 of the differentiation protocol.

In some embodiments, the method induces differentiation of the pluripotent stem cells into astrocytes. Any available and known method for inducing differentiation of the pluripotent stem cells into astrocytes can be used. Exemplary methods of inducing differentiation of pluripotent stem cells into astrocytes can be found in, e.g., TCW et al., Stem Cell Reports (2017), Vol. 9: 600-614, including the methods described in the references cited therein, e.g., in Table 1. Exemplary methods of inducing differentiation of pluripotent stem cells into astrocytes can include, in some embodiments, the use of commercially available kits, and provided methods for use of such kits, including, e.g., Astrocyte Medium, Catalog #1801 (ScienCell Research Laboratories, Carlsbad, CA); Astrocyte Medium, Catalog #A1261301 (ThermoFisher Scientific Inc, Waltham, MA); and AGM Astrocyte Growth Medium BulletKit, Catalog #CC-3186 (Lonza, Basel, Switzerland).

In some embodiments, the method induces differentiation of the pluripotent stem cells into oligodendrocytes. Any available and known method for inducing differentiation of the pluripotent stem cells into oligodendrocytes can be used. Exemplary methods of inducing differentiation of pluripotent stem cells into oligodendrocytes can be found in, e.g., Ehrlich et al., PNAS (2017), Vol. 114(11): E2243-E2252; Douvaras et al., Stem Cell Reports (2014), Vol. 3(2): 250-259; Stacpoole et al., Stem Cell Reports (2013), Vol. 1(5): 437-450; Wang et al., Cell Stem Cell (2013), Vol. 12(2): 252-264; and Piao et al., Cell Stem Cell (2015), Vol. 16(2): 198-210.

In some embodiments, the test cells are from an in vitro population of stem-cell derived GABAergic neuronal cells. Exemplary methods for differentiating stem-cell derived GABAergic neuronal cells in vitro are described in Maroof et al. (2013) Cell Stem Cell 12(5): 573-586. US 2020/0002679A1, US20110183912A1, and US20140248696A1. In some embodiments, the first, second, third, and/or control differentiation states are that of GABAergic neuronal precursor cells, committed GABAergic neuronal cells, or determined GABAergic neuronal cells. In some embodments, the first differentiation state is that of GAB Aergic neuronal precursor cells; the second differentiation state is that of determined GABAergic neuronal cells; and the third differentiation state is that of committed GAB Aergic neuronal cells. In some embodiments, the cells identified as having the desired differentiation state, e.g., having the second differentiation state, can be used for the treatment of epilepsy.

In some embodiments, the test cells are from an in vitro population from a culture of cells differentiated from pluripotent cells that are subjected to a differentiation protocol for inducing the differentiation of PSCs, e.g., iPSCs, into inhibitory neurons, e.g., GABAergic neuronal cells. Exemplary methods for differentiating inhibitory neurons in vitro are described in Kang et al. (2017) Sci Rep 7:12233 and Nicholas et al. (2013) Cell Stem Cell 12(5):573-86. In some embodiments, cells of the second differentiation state are in any of weeks 5-10 of the differentiation protocol. In some embodiments, cells of the first differentiation state are at week 4 or earlier, week 3 or earlier, or week 2 or earlier of the differentiation protocol. In some embodiments, cells of the third differentiation state are at week 12 or later, week 14 or later, week 16 or later, week 18 or later, or week 20 or later of the differentiation protocol. In some embodiments, cells of the second differentiation state are in any of weeks 5-10 of the differentiation protocol; and cells of the third differentiation state are at week 12 or later, week 14 or later, week 16 or later, week 18 or later, or week 20 or later of the differentiation protocol. In some embodiments, cells of the first differentiation state are at week 4 or earlier, week 3 or earlier, or week 2 or earlier of the differentiation protocol; cells of the second differentiation state are in any of weeks 5-10 of the differentiation protocol; and cells of the third differentiation state are at week 12 or later, week 14 or later, week 16 or later, week 18 or later, or week 20 or later of the differentiation protocol. In some embodiments, cells of the third differentiation state are in any of weeks 20-30 of the differentiation protocol.

In some embodiments, the method induces the differentiation of iPSCs into floor plate midbrain precursor cells, determined dopaminergic cells, and/or dopaminergic neurons. In some embodiments, the method involves (a) performing a first incubation including culturing pluripotent stem cells in a non-adherent culture vessel under conditions to produce a cellular spheroid, wherein beginning at the initiation of the first incubation (day 0) the cells are exposed to (i) an inhibitor of TGF-β/activing-Nodal signaling; (ii) at least one activator of Sonic Hedgehog (SHH) signaling; (iii) an inhibitor of bone morphogenetic protein (BMP) signaling; and (iv) an inhibitor of glycogen synthase kinase 3β (GSK3β) signaling; and (b) performing a second incubation including culturing cells of the spheroid in a substrate-coated culture vessel under conditions to neurally differentiate the cells.

In some embodiments, the method involves exposing pluripotent stem cells to (a) an inhibitor of bone morphogenetic protein (BMP) signaling; (b) an inhibitor of TGF-β/activing-Nodal signaling; and (c) at least one activator of Sonic Hedgehog (SHH) signaling. In some embodiments, the method further includes exposing the pluripotent stem cells to at least one inhibitor of GSK3β signaling. In some embodiments, the exposing to an inhibitor of BMP signaling and the inhibitor of TGF-β/activing-Nodal signaling occurs while the pluripotent stem cells are attached to a substrate. In some embodiments, during the exposing to the inhibitor of BMP signaling, the inhibitor of TGF-β/activing-Nodal signaling, and the at least one activator of SHH signaling, the pluripotent stem cells are attached to a substrate. In some embodiments, during the exposing to the at least one inhibitor of GSK3β signaling, the pluripotent stem cells are attached to a substrate. In some embodiments, during the exposing to the inhibitor of BMP signaling, the inhibitor of TGF-β/activing-Nodal signaling, and the at least one activator of SHH signaling, the pluripotent stem cells are in a non-adherent culture vessel under conditions to produce a cellular spheroid. In some embodiments, during the exposing to the at least one inhibitor of GSK3β signaling, the pluripotent stem cells are in a non-adherent culture vessel under conditions to produce a cellular spheroid.

In some embodiments, a non-adherent culture vessel allows for three-dimensional formation of cell aggregates. In some embodiments, iPSCs are cultured in a non-adherent culture vessel, such as a multi-well plate, to produce cell aggregates (e.g., spheroids). In some embodiments, iPSCs are cultured in a non-adherent culture vessel, such as a multi-well plate, to produce cell aggregates (e.g., spheroids) on about day 7 of the method. In some embodiments, the cell aggregate (e.g., spheroid) expresses at least one of PAX6 and OTX2 on or by about day 7 of the method.

In some embodiments, the first incubation is from about day 0 through about day 6. In some embodiments, the first incubation comprises culturing pluripotent stem cells in a culture media (“media”). In some embodiments, the first incubation comprises culturing pluripotent stem cells in the media from about day 0 through about day 6. In some embodiments, the first incubation comprises culturing pluripotent stem cells in the media to induce differentiation of the PSCs into floor plate midbrain precursor cells.

In some embodiments, the media is also supplemented with a serum replacement containing minimal non-human-derived components (e.g., KnockOut™ serum replacement). In some embodiments, the serum replacement is provided in the media at 5% (v/v) for at least a portion of the first incubation. In some embodiments, the serum replacement is provided in the media at 5% (v/v) on day 0 and day 1. In some embodiments, the serum replacement is provided in the media at 2% (v/v) for at least a portion of the first incubation. In some embodiments, the serum replacement is provided in the media at 2% (v/v) from day 2 through day 6. In some embodiments, the serum replacement is provided in the media at 5% (v/v) on day 0 and day 1, and at 2% (v/v) from day 2 through day 6.

In some embodiments, the media is further supplemented with small molecules, such as any described above. In some embodiments, the small molecules are selected from among the group consisting of: a Rho-associated protein kinase (ROCK) inhibitor, an inhibitor of TGF-β/activing-Nodal signaling, at least one activator of Sonic Hedgehog (SHH) signaling, an inhibitor of bone morphogenetic protein (BMP) signaling, an inhibitor of glycogen synthase kinase 3β (GSK3β) signaling, and combinations thereof.

In some embodiments the media is supplemented with a Rho-associated protein kinase (ROCK) inhibitor on one or more days when cells are passaged. In some embodiments the media is supplemented with a ROCK inhibitor each day that cells are passaged. In some embodiments the media is supplemented with a ROCK inhibitor on day 0.

In some embodiments, the ROCK inhibitor is selected from among the group consisting of: Fasudil, Ripasudil, Netarsudil, RKI-1447, Y-27632, GSK429286A, Y-30141, and combinations thereof. In some embodiments, the ROCK inhibitor is a small molecule. In some embodiments, the ROCK inhibitor selectively inhibits p160ROCK. In some embodiments, the ROCK inhibitor is Y-27632, having the formula:

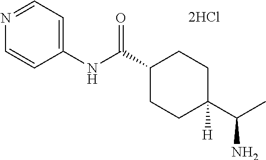 [SMILES: C[C@@H](N)[C@H]1CC[C@H](C(=O)Nc2ccncc2)CC1]

In some embodiments the media is supplemented with an inhibitor of TGF-β/activing-Nodal signaling. In some embodiments the media is supplemented with an inhibitor of TGF-β/activing-Nodal signaling up to about day 7 (e.g. day 6 or day 7). In some embodiments the media is supplemented with an inhibitor of TGF-β/activing-Nodal signaling from about day 0 through day 6, each day inclusive.

In some embodiments, the inhibitor of TGF-β/activing-Nodal signaling is a small molecule. In some embodiments, the inhibitor of TGF-β/activing-Nodal signaling is capable of lowering or blocking transforming growth factor beta (TGFβ)/Activin-Nodal signaling. In some embodiments, the inhibitor of TGF-β/activing-Nodal signaling inhibits ALK4, ALK5, ALK7, or combinations thereof. In some embodiments, the inhibitor of TGF-β/activing-Nodal signaling inhibits ALK4, ALK5, and ALK7. In some embodiments, the inhibitor of TGF-β/activing-Nodal signaling does not inhibit ALK2, ALK3, ALK6, or combinations thereof. In some embodiments, the inhibitor does not inhibit ALK2, ALK3, or ALK6. In some embodiments, the inhibitor of TGF-β/activing-Nodal signaling is SB431542 (e.g., CAS 301836-41-9, molecular formula of C22H18N4O3, and name of 4-[4-(1,3-benzodioxol-5-yl)-5-(2-pyridinyl)-1H-imidazol-2-yl]-benzamide), having the formula:

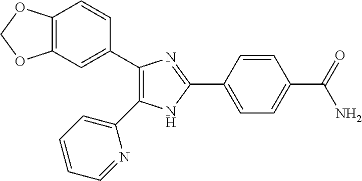 [SMILES: NC(=O)c1ccc(-c2nc(-c3ccc4c(c3)OCO4)c(-c3ccccn3)[nH]2)cc1]

In some embodiments, the inhibitor of TGF-β/activing-Nodal signaling is a small molecule. In some embodiments, the inhibitor of TGF-β/activing-Nodal signaling is capable of lowering or blocking transforming growth factor beta (TGFβ)/Activin-Nodal signaling. In some embodiments, the inhibitor of TGF-β/activing-Nodal signaling inhibits ALK4, ALK5, ALK7, or combinations thereof. In some embodiments, the inhibitor of TGF-β/activing-Nodal signaling inhibits ALK4, ALK5, and ALK7. In some embodiments, the inhibitor of TGF-β/activing-Nodal signaling does not inhibit ALK2, ALK3, ALK6, or combinations thereof. In some embodiments, the inhibitor does not inhibit ALK2, ALK3, or ALK6. In some embodiments, the inhibitor of TGF-β/activing-Nodal signaling is SB431542 (e.g., CAS 301836-41-9, molecular formula of C22H18N4O3, and name of 4-[4-(1,3-benzodioxol-5-yl)-5-(2-pyridinyl)-1H-imidazol-2-yl]-benzamide), having the formula:

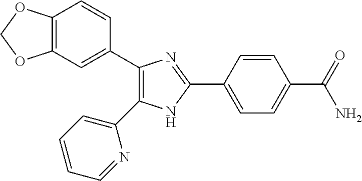 [SMILES: NC(=O)c1ccc(-c2nc(-c3ccc4c(c3)OCO4)c(-c3ccccn3)[nH]2)cc1]

In some embodiments, the at least one activator of SHH signaling is an activator of the Hedgehog receptor Smoothened. It some embodiments, the at least one activator of SHH signaling is a small molecule. In some embodiments, the least one activator of SHH signaling is purmorphamine (e.g. CAS 483367-10-8), having the formula below:

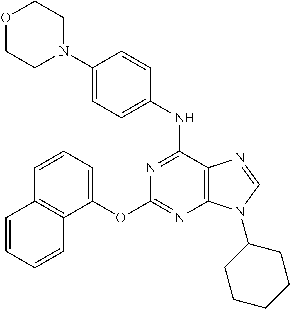 [SMILES: c1ccc2c(Oc3nc(Nc4ccc(N5CCOCC5)cc4)c4ncn(C5CCCCC5)c4n3)cccc2c1]

In some embodiments, cells are exposed to purmorphamine at a concentration of about 10 μM. In some embodiments, cells are exposed to purmorphamine at a concentration of about 10 μM up to day 7 (e.g., day 6 or day 7). In some embodiments, cells are exposed to purmorphamine at a concentration of about 10 μM from about day 0 through about day 6, inclusive of each day.

In some embodiments, the at least one activator of SHH signaling is SHH protein and purmorphamine. In some embodiments, cells are exposed to SHH protein and purmorphamine at a concentration up to about day 7 (e.g., day 6 or day 7). In some embodiments, cells are exposed to SHH protein and purpomorphamine from about day 0 through about day 6, inclusive of each day. In some embodiments, cells are exposed to 100 ng/mL SHH protein and 10 μM purmorphamine at a concentration up to about day 7 (e.g., day 6 or day 7). In some embodiments, cells are exposed to 100 ng/mL SHH protein and 10 μM purpomorphamine from about day 0 through about day 6, inclusive of each day.

In some embodiments the media is supplemented with an inhibitor of BMP signaling. In some embodiments the media is supplemented with an inhibitor of BMP signaling up to about day 7 (e.g., day 6 or day 7). In some embodiments the media is supplemented with an inhibitor of BMP signaling from about day 0 through day 6, each day inclusive.

In some embodiments, the inhibitor of BMP signaling is a small molecule. In some embodiments, the inhibitor of BMP signaling is selected from LDN193189 or K02288. In some embodiments, the inhibitor of BMP signaling is capable of inhibiting “Small Mothers Against Decapentaplegic” SMAD signaling. In some embodiments, the inhibitor of BMP signaling inhibits ALK1, ALK2, ALK3, ALK6, or combinations thereof. In some embodiments, the inhibitor of BMP signaling inhibits ALK1, ALK2, ALK3, and ALK6. In some embodiments, the inhibitor of BMP signaling inhibits BMP2, BMP4, BMP6, BMP7, and Activin cytokine signals and subsequently SMAD phosphorylation of Smad1, Smad5, and Smad8. In some embodiments, the inhibitor of BMP signaling is LDN193189. In some embodiments, the inhibitor of BMP signaling is LDN193189 (e.g., IUPAC name 4-(6-(4-(piperazin-1-yl)phenyl)pyrazolo[1,5-a]pyrimidin-3-yl)quinoline, with a chemical formula of C25H22N6), having the formula:

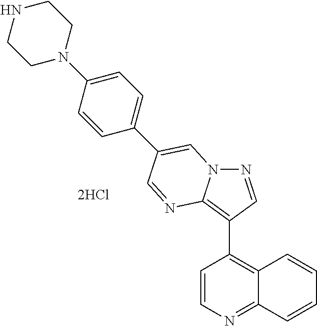 [SMILES: c1ccc2c(-c3cnn4cc(-c5ccc(N6CCNCC6)cc5)cnc34)ccnc2c1]

In some embodiments, cells are exposed to LDN193189 at a concentration of about 0.1 μM. In some embodiments, cells are exposed to LDN193189 at a concentration of about 0.1 μM up to about day 7 (e.g., day 6 or day 7). In some embodiments, cells are exposed to LDN193189 at a concentration of about 0.1 μM from about day 0 through about day 6, inclusive of each day.

In some embodiments the media is supplemented with an inhibitor of GSK3β signaling. In some embodiments the media is supplemented with an inhibitor of GSK3β signaling up to about day 7 (e.g., day 6 or day 7). In some embodiments the media is supplemented with an inhibitor of GSK3β signaling from about day 0 through day 6, each day inclusive.

In some embodiments, the inhibitor of GSK3β signaling is selected from among the group consisting of: lithium ion, valproic acid, iodotubercidin, naproxen, famotidine, curcumin, olanzapine, CHIR99012, and combinations thereof. In some embodiments, the inhibitor of GSK3β signaling is a small molecule. In some embodiments, the inhibitor of GSK3β signaling inhibits a glycogen synthase kinase 3β enzyme. In some embodiments, the inhibitor of GSK3β signaling inhibits GSK3a. In some embodiments, the inhibitor of GSK3β signaling modulates TGF-β and MAPK signaling. In some embodiments, the inhibitor of GSK3β signaling is an agonist of wingless/integrated (Wnt) signaling. In some embodiments, the inhibitor of GSK3β signaling has an IC50=6.7 nM against human GSK3β. In some embodiments, the inhibitor of GSK3β signaling is CHIR99021 (e.g., “3-[3-(2-Carboxyethyl)-4-methylpyrrol-2-methylidenyl]-2-indolinone” or IUPAC name 6-(2-(4-(2,4-dichlorophenyl)-5-(4-methyl-1H-imidazol-2-yl)pyrimidin-2-ylamino)ethylamino)nicotinonitrile), having the formula:

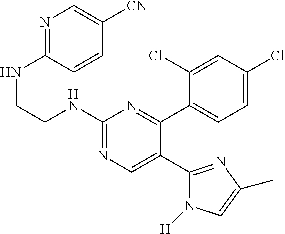 [SMILES: Cc1c[nH]c(-c2cnc(NCCNc3ccc(C#N)cn3)nc2-c2ccc(Cl)cc2Cl)n1]

In some embodiments, cells are exposed to CHIR99021 at a concentration of about 2.0 μM. In some embodiments, cells are exposed to CHIR99021 at a concentration of about 2.0 μM up to about day 7 (e.g., day 6 or day 7). In some embodiments, cells are exposed to CHIR99021 at a concentration of about 2.0 μM from about day 0 through about day 6, inclusive of each day.

In some embodiments, from day about 2 to about day 6, at least about 50% of the media is replaced daily. In some embodiments, from about day 2 to about day 6, about 50% of the media is replaced daily, every other day, or every third day. In some embodiments, from about day 2 to about day 6, about 50% of the media is replaced daily. In some embodiments, at least about 75% of the media is replaced on day 1. In some embodiments, about 100% of the media is replaced on day 1. In some embodiments, the replacement media contains small molecules about twice as concentrated as compared to the concentration of the small molecules in the media on day 0.

In some embodiments, the first incubation comprises culturing pluripotent stem cells in a “basal induction media.” In some embodiments, the first incubation comprises culturing pluripotent stem cells in the basal induction media from about day 0 through about day 6. In some embodiments, the first incubation comprises culturing pluripotent stem cells in the basal induction media to induce differentiation of the PSCs into floor plate midbrain precursor cells.

In some embodiments, the basal induction media is formulated to contain Neurobasal™ media and DMEM/F12 media at a 1:1 ratio, supplemented with N-2 and B27 supplements, non-essential amino acids (NEAA), GlutaMAX™, L-glutamine, β-mercaptoethanol, and insulin. In some embodiments, the basal induction media is further supplemented with any of the small molecules as described above.

In some embodiments, cell aggregates (e.g. spheroids) that are produced following the first incubation of culturing pluripotent stem cells in a non-adherent culture vessel are transferred or dissociated, prior to carrying out a second incubation of the cells on a substrate (adherent culture).

In some embodiments, the first incubation is carried out to produce a cell aggregate (e.g. a spheroid) that expresses at least one of PAX6 and OTX2. In some embodiments, the first incubation produces a cell aggregate (e.g. a spheroid) that expresses PAX6 and OTX2. In some embodiments, the first incubation produces a cell aggregate (e.g. a spheroid) on or by about day 7 of the methods. In some embodiments, the first incubation produces a cell aggregate (e.g. a spheroid) that expresses at least one of PAX6 and OTX2 on or by about day 7 of the methods. In some embodiments, the first incubation produces a cell aggregate (e.g. a spheroid) that expresses PAX6 and OTX2 on or by about day 7 of the methods.

In some embodiments, the cell aggregate (e.g. spheroid) produced by the first incubation is dissociated prior to the second incubation of the cells on a substrate. In some embodiments, the cell aggregate (e.g. spheroid) produced by the first incubation is dissociated to produce a cell suspension. In some embodiments, the cell suspension produced by the dissociation is a single cell suspension. In some embodiments, the dissociation is carried out at a time when the spheroid cells express at least one of PAX6 and OTX2. In some embodiments, the dissociation is carried out at a time when the spheroid cells express PAX6 and OTX2. In some embodiments, the dissociation is carried out on about day 7. In some embodiments, the cell aggregate (e.g. spheroid) is dissociated by enzymatic dissociation. In some embodiments, the enzyme is selected from among the group consisting of: accutase, dispase, collagenase, and combinations thereof. In some embodiments, the enzyme comprises accutase. In some embodiments, the enzyme is accutase. In some embodiments, the enzyme is dispase. In some embodiments, the enzyme is collagenase.

In some embodiments, the cell aggregate or cell suspension produced therefrom is transferred to a substrate-coated culture vessel for a second incubation. In some embodiments, the cell aggregate (e.g. spheroid) or cell suspension produced therefrom is transferred to a substrate-coated culture vessel following dissociation of the cell aggregate (e.g. spheroid). In some embodiments, the transferring is carried out immediately after the dissociating. In some embodiments, the transferring is carried out on about day 7.

In some embodiments, the cell aggregate (e.g., spheroid) is not dissociated prior to a second incubation. In some embodiments, a cell aggregate (e.g. spheroid) is transferred in its entirety to a substrate-coated culture vessel for a second incubation. In some embodiments, the transferring is carried out at a time when the spheroid cells express at least one of PAX6 and OTX2. In some embodiments, the transferring is carried out at a time when the spheroid cells express PAX6 and OTX2. In some embodiments, the transferring is carried out on about day 7.

In some embodiments, the second incubation involves culturing cells of the spheroid in a culture vessel coated with a substrate including laminin, collagen, entactin, heparin sulfate proteoglycans, or a combination thereof, wherein beginning on day 7, the cells are exposed to (i) an inhibitor of BMP signaling and (ii) an inhibitor of GSK3β signaling; and beginning on day 11, the cells are exposed to (i) brain-derived neurotrophic factor (BDNF); (ii) ascorbic acid; (iii) glial cell-derived neurotrophic factor (GDNF); (iv) dibutyryl cyclic AMP (dbcAMP); (v) transforming growth factor beta-3 (TGFβ3); and (vi) an inhibitor of Notch signaling. In some embodiments, the method further includes harvesting the differentiated cells.

In some embodiments, the substrate-coated culture vessel is a culture vessel with a surface to which cells can attach. In some embodiments, the substrate-coated culture vessel is a culture vessel with a surface to which a substantial number of cells attach. In some embodiments, the substrate is a basement membrane protein. In some embodiments, the substrate is laminin, collagen, entactin, heparin sulfate proteoglycans, or a combination thereof. In some embodiments, the substrate is laminin. In some embodiments, the substrate is collagen. In some embodiments, the substrate is entactin. In some embodiments, the substrate is heparin sulfate proteoglycans. In some embodiments, the substrate is a recombinant protein. In some embodiments, the substrate is recombinant laminin. In some embodiments, the substrate-coated culture vessel is exposed to poly-L-ornithine. In some embodiments, the substrate-coated culture vessel is exposed to poly-L-ornithine prior to being used for cell culture.

In some embodiments, the substrate-coated culture vessel allows for a monolayer cell culture. In some embodiments, cells derived from the cell aggregate (e.g. spheroid) produced by the first incubation are cultured in a monolayer culture on the substrate-coated plates. In some embodiments, cells derived from the cell aggregate (e.g. spheroid) produced by the first incubation are cultured to produce a monolayer culture of cells positive for one or more of LMX1A, FOXA2, EN1, CORIN, and combinations thereof. In some embodiments, cells derived from the cell aggregate (e.g. spheroid) produced by the first incubation are cultured to produce a monolayer culture of cells, wherein at least some of the cells are positive for EN1 and CORIN. In some embodiments, cells derived from the cell aggregate (e.g. spheroid) produced by the first incubation are cultured to produce a monolayer culture of cells, wherein at least some of the cells are TH+. In some embodiments, at least some cells are TH+ by or on about day 25. In some embodiments, cells derived from the cell aggregate (e.g. spheroid) produced by the first incubation are cultured to produce a monolayer culture of cells, wherein at least some of the cells are TH+FOXA2+. In some embodiments, at least some cells are TH+FOXA2+ by or on about day 25.

In the method, the second incubation involves culturing cells of the spheroid in a substrate-coated culture vessel under conditions to induce neural differentiation of the cells. In some embodiments, the cells of the spheroid are plated on the substrate-coated culture vessel on about day 7.

In some embodiments, the second incubation is from about day 7 until harvesting of the cells. In some embodiments, the cells are harvested on about day 16 or later. In some embodiments, the cells are harvested between about day 16 and about day 30. In some embodiments, the cells are harvested between about day 18 and about day 25. In some embodiments, the cells are harvested on about day 18. In some embodiments, the cells are harvested on about day 25. In some embodiments, the second incubation is from about day 7 until about day 18. In some embodiments, the second incubation is from about day 7 until about day 25.

In some embodiments, the second incubation involves culturing cells derived from the cell aggregate (e.g. spheroid) in a culture media (“media”).

In some embodiments, the second incubation involves culturing the cells in the media from about day 7 until harvest or collection. In some embodiments, cells are cultured in the media to produce determined dopaminergic cells, or dopaminergic neurons.

In some embodiments, the media is also supplemented with a serum replacement containing minimal non-human-derived components (e.g., KnockOut™ serum replacement). In some embodiments, the media is supplemented with the serum replacement from about day 7 through about day 10. In some embodiments, the media is supplemented with about 2% (v/v) of the serum replacement. In some embodiments, the media is supplemented with about 2% (v/v) of the serum replacement from about day 7 through about day 10.

In some embodiments, the media is further supplemented with small molecules. In some embodiments, the small molecules are selected from among the group consisting of: a Rho-associated protein kinase (ROCK) inhibitor, an inhibitor of bone morphogenetic protein (BMP) signaling, an inhibitor of glycogen synthase kinase 3β (GSK3β) signaling, and combinations thereof.

In some embodiments the media is supplemented with a Rho-associated protein kinase (ROCK) inhibitor on one or more days when cells are passaged. In some embodiments the media is supplemented with a ROCK inhibitor each day that cells are passaged. In some embodiments the media is supplemented with a ROCK inhibitor on day 7, day 16, day 20, or a combination thereof. In some embodiments the media is supplemented with a ROCK inhibitor on day 7. In some embodiments the media is supplemented with a ROCK inhibitor on day 16. In some embodiments the media is supplemented with a ROCK inhibitor on day 20. In some embodiments the media is supplemented with a ROCK inhibitor on day 7 and day 16. In some embodiments the media is supplemented with a ROCK inhibitor on day 16 and day 20. In some embodiments the media is supplemented with a ROCK inhibitor on day 7, day 16, and day 20.

In some embodiments, the ROCK inhibitor is Fasudil, Ripasudil, Netarsudil, RKI-1447, Y-27632, GSK429286A, Y-30141, or a combination thereof. In some embodiments, the ROCK inhibitor is a small molecule. In some embodiments, the ROCK inhibitor selectively inhibits p160ROCK. In some embodiments, the ROCK inhibitor is Y-27632, having the formula:

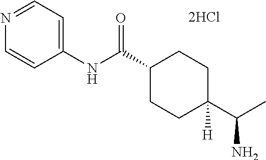 [SMILES: C[C@@H](N)[C@H]1CC[C@H](C(=O)Nc2ccncc2)CC1]

In some embodiments, cells are exposed to Y-27632 at a concentration of about 10 μM. In some embodiments, cells are exposed to Y-27632 at a concentration of about 10 μM on day 7, day 16, day 20, or a combination thereof. In some embodiments, cells are exposed to Y-27632 at a concentration of about 10 μM on day 7. In some embodiments, cells are exposed to Y-27632 at a concentration of about 10 μM on day 16. In some embodiments, cells are exposed to Y-27632 at a concentration of about 10 μM on day 20. In some embodiments, cells are exposed to Y-27632 at a concentration of about 10 μM on day 7 and day 16. In some embodiments, cells are exposed to Y-27632 at a concentration of about 10 μM on day 16 and day 20. In some embodiments, cells are exposed to Y-27632 at a concentration of about 10 μM on day 7, day 16, and day 20.

In some embodiments the media is supplemented with an inhibitor of BMP signaling. In some embodiments the media is supplemented with an inhibitor of BMP signaling from about day 7 up to about day 11 (e.g., day 10 or day 11). In some embodiments the media is supplemented with an inhibitor of BMP signaling from about day 7 through day 10, each day inclusive.

In some embodiments, the inhibitor of BMP signaling is a small molecule. In some embodiments, the inhibitor of BMP signaling is LDN193189 or K02288. In some embodiments, the inhibitor of BMP signaling is capable of inhibiting “Small Mothers Against Decapentaplegic” SMAD signaling. In In some embodiments, the inhibitor of BMP signaling inhibits ALK1, ALK2, ALK3, ALK6, or combinations thereof. In some embodiments, the inhibitor of BMP signaling inhibits ALK1, ALK2, ALK3, and ALK6. In some embodiments, the inhibitor of BMP signaling inhibits BMP2, BMP4, BMP6, BMP7, and Activin cytokine signals and subsequently SMAD phosphorylation of Smad1, Smad5, and Smad8. In some embodiments, the inhibitor of BMP signaling is LDN193189. In some embodiments, the inhibitor of BMP signaling is LDN193189 (e.g., IUPAC name 4-(6-(4-(piperazin-1-yl)phenyl)pyrazolo[1,5-a]pyrimidin-3-yl)quinoline, with a chemical formula of C25H22N6), having the formula:

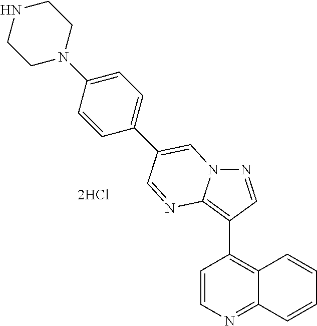 [SMILES: c1ccc2c(-c3cnn4cc(-c5ccc(N6CCNCC6)cc5)cnc34)ccnc2c1]

In some embodiments, cells are exposed to LDN193189 at a concentration of about 0.1 μM. In some embodiments, cells are exposed to LDN193189 at a concentration of about 0.1 μM from about day 7 up to about day 11 (e.g., day 10 or day 11). In some embodiments, cells are exposed to LDN193189 at a concentration of about 0.1 μM from about day 7 through about day 10, inclusive of each day.

In some embodiments the media is supplemented with an inhibitor of GSK3β signaling. In some embodiments the media is supplemented with an inhibitor of GSK3β signaling from about day 7 up to about day 13 (e.g., day 12 or day 13). In some embodiments the media is supplemented with an inhibitor of GSK3β signaling from about day 7 through day 12, each day inclusive.

In some embodiments, the inhibitor of GSK3β signaling is selected from lithium ion, valproic acid, iodotubercidin, naproxen, famotidine, curcumin, olanzapine, CHIR99012, or a combination thereof. In some embodiments, the inhibitor of GSK3β signaling is a small molecule. In some embodiments, the inhibitor of GSK3β signaling inhibits a glycogen synthase kinase 3β enzyme. In some embodiments, the inhibitor of GSK3β signaling inhibits GSK3a. In some embodiments, the inhibitor of GSK3β signaling modulates TGF-β and MAPK signaling. In some embodiments, the inhibitor of GSK3β signaling is an agonist of wingless/integrated (Wnt) signaling. In some embodiments, the inhibitor of GSK3β signaling has an IC50=6.7 nM against human GSK3β. In some embodiments, the inhibitor of GSK3β signaling is CHIR99021 (e.g., “3-[3-(2-Carboxyethyl)-4-methylpyrrol-2-methylidenyl]-2-indolinone” or IUPAC name 6-(2-(4-(2,4-dichlorophenyl)-5-(4-methyl-1H-imidazol-2-yl)pyrimidin-2-ylamino)ethylamino)nicotinonitrile), having the formula:

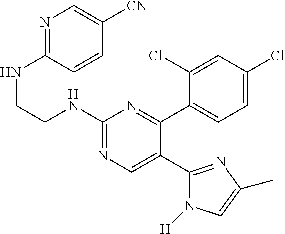 [SMILES: Cc1c[nH]c(-c2cnc(NCCNc3ccc(C#N)cn3)nc2-c2ccc(Cl)cc2Cl)n1]

In some embodiments, cells are exposed to CHIR99021 at a concentration of about 2.0 μM. In some embodiments, cells are exposed to CHIR99021 at a concentration of about 2.0 μM from about day 7 up to about day 13 (e.g., day 12 or day 13). In some embodiments, cells are exposed to CHIR99021 at a concentration of about 2.0 μM from about day 7 through about day 12, inclusive of each day.

In some embodiments the media is supplemented with brain-derived neurotrophic factor (BDNF). In some embodiments the media is supplemented with BDNF beginning on about day 11. In some embodiments the media is supplemented with BDNF from about day 11 until harvest or collection. In some embodiments the media is supplemented with BDNF from about day 11 through day 18. In some embodiments the media is supplemented with BDNF from about day 11 through day 25.

In some embodiments, the media is supplemented with about 20 ng/mL BDNF beginning on about day 11. In some embodiments the media is supplemented with 20 ng/mL BDNF from about day 11 until harvest or collection. In some embodiments the media is supplemented with about 20 ng/mL BDNF from about day 11 through day 18. In some embodiments the media is supplemented with about 20 ng/mL BDNF from about day 11 through day 25.

In some embodiments the media is supplemented with glial cell-derived neurotrophic factor (GDNF). In some embodiments the media is supplemented with GDNF beginning on about day 11. In some embodiments the media is supplemented with GDNF from about day 11 until harvest or collection. In some embodiments the media is supplemented with GDNF from about day 11 through day 18. In some embodiments the media is supplemented with GDNF from about day 11 through day 25.

In some embodiments, the media is supplemented with about 20 ng/mL GDNF beginning on about day 11. In some embodiments the media is supplemented with 20 ng/mL GDNF from about day 11 until harvest or collection. In some embodiments the media is supplemented with about 20 ng/mL GDNF from about day 11 through day 18. In some embodiments the media is supplemented with about 20 ng/mL GDNF from about day 11 through day 25.

In some embodiments the media is supplemented with ascorbic acid. In some embodiments the media is supplemented with ascorbic acid beginning on about day 11. In some embodiments the media is supplemented with ascorbic acid from about day 11 until harvest or collection. In some embodiments the media is supplemented with ascorbic acid from about day 11 through day 18. In some embodiments the media is supplemented with ascorbic acid from about day 11 through day 25.

In some embodiments, the media is supplemented with about 0.2 mM ascorbic acid beginning on about day 11. In some embodiments the media is supplemented with 0.2 mM ascorbic acid from about day 11 until harvest or collection. In some embodiments the media is supplemented with about 0.2 mM ascorbic acid from about day 11 through day 18. In some embodiments the media is supplemented with about 0.2 mM ascorbic acid from about day 11 through day 25.

In some embodiments, the media is supplemented with dibutyryl cyclic AMP (dbcAMP). In some embodiments the media is supplemented with dbcAMP beginning on about day 11. In some embodiments the media is supplemented with dbcAMP from about day 11 until harvest or collection. In some embodiments the media is supplemented with dbcAMP from about day 11 through day 18. In some embodiments the media is supplemented with dbcAMP from about day 11 through day 25.

In some embodiments, the media is supplemented with about 0.5 mM dbcAMP beginning on about day 11. In some embodiments the media is supplemented with 0.5 mM dbcAMP from about day 11 until harvest or collection. In some embodiments the media is supplemented with about 0.5 mM dbcAMP from about day 11 through day 18. In some embodiments the media is supplemented with about 0.5 mM dbcAMP from about day 11 through day 25.

In some embodiments, the media is supplemented with transforming growth factor beta 3 (TGFβ3). In some embodiments the media is supplemented with TGFβ3 beginning on about day 11. In some embodiments the media is supplemented with TGFβ3 from about day 11 until harvest or collection. In some embodiments the media is supplemented with TGFβ3 from about day 11 through day 18. In some embodiments the media is supplemented with TGFβ3 from about day 11 through day 25.

In some embodiments, the media is supplemented with about 1 ng/mL TGFβ3 beginning on about day 11. In some embodiments the media is supplemented with 1 ng/mL TGFβ3 from about day 11 until harvest or collection. In some embodiments the media is supplemented with about 1 ng/mL TGFβ3 from about day 11 through day 18. In some embodiments the media is supplemented with about 1 ng/mL TGFβ3 from about day 11 through day 25.

In some embodiments the media is supplemented with an inhibitor of Notch signaling. In some embodiments the media is supplemented with an inhibitor of Notch signaling beginning on about day 11. In some embodiments the media is supplemented with an inhibitor of Notch signaling from about day 11 until harvest or collection. In some embodiments the media is supplemented with an inhibitor of Notch signaling from about day 11 through day 18. In some embodiments the media is supplemented with an inhibitor of Notch signaling from about day 11 through day 25.

In some embodiments, an inhibitor of Notch signaling is selected from cowanin, PF-03084014, L685458, LY3039478, DAPT, or a combination thereof. In some embodiments, the inhibitor of Notch signaling inhibits gamma secretase. In some embodiments, the inhibitor of Notch signaling is a small molecule. In some embodiments, the inhibitor of Notch signaling is DAPT, having the following formula:

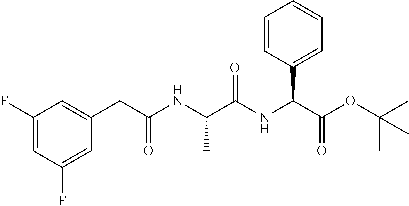 [SMILES: C[C@H](NC(=O)Cc1cc(F)cc(F)c1)C(=O)N[C@H](C(=O)OC(C)(C)C)c1ccccc1]

In some embodiments, the media is supplemented with about 10 μM DAPT beginning on about day 11. In some embodiments the media is supplemented with 10 μM DAPT from about day 11 until harvest or collection. In some embodiments the media is supplemented with about 10 μM DAPT from about day 11 through day 18. In some embodiments the media is supplemented with about 10 μM DAPT from about day 11 through day 25.

In some embodiments, beginning on about day 11, the media is supplemented with about 20 ng/mL BDNF, about 20 ng/mL GDNF, about 0.2 mM ascorbic acid, about 0.5 mM dbcAMP, about 1 ng/mL TGFβ3, and about 10 μM DAPT. In some embodiments, from about day 11 until harvest or collection, the media is supplemented with about 20 ng/mL BDNF, about 20 ng/mL GDNF, about 0.2 mM ascorbic acid, about 0.5 mM dbcAMP, about 1 ng/mL TGFβ3, and about 10 μM DAPT. In some embodiments, from about day 11 until day 18, the media is supplemented with about 20 ng/mL BDNF, about 20 ng/mL GDNF, about 0.2 mM ascorbic acid, about 0.5 mM dbcAMP, about 1 ng/mL TGFβ3, and about 10 μM DAPT. In some embodiments, from about day 11 until day 25, the media is supplemented with about 20 ng/mL BDNF, about 20 ng/mL GDNF, about 0.2 mM ascorbic acid, about 0.5 mM dbcAMP, about 1 ng/mL TGFβ3, and about 10 μM DAPT.

In some embodiments, a serum replacement is provided in the media from about day 7 through about day 10. In some embodiments, the serum replacement is provided at 2% (v/v) in the media on day 7 through day 10.

In some embodiments, from day about 7 to about day 16, at least about 50% of the media is replaced daily. In some embodiments, from about day 7 to about day 16, about 50% of the media is replaced daily, every other day, or every third day. In some embodiments, from about day 7 to about day 16, about 50% of the media is replaced daily. In some embodiments, beginning on about day 17, at least about 50% of the media is replaced daily, every other day, or every third day. In some embodiments, beginning on about day 17, at least about 50% of the media is replaced every other day. In some embodiments, beginning on about day 17, about 50% of the media is replaced daily, every other day, or every third day. In some embodiments, beginning on about day 17, about 50% of the media is replaced every other day. In some embodiments, the replacement media contains small molecules about twice as concentrated as compared to the concentration of the small molecules in the media on day 0.

In some embodiments, the second incubation involves culturing cells derived from the cell aggregate (e.g. spheroid) in a “basal induction media.” In some embodiments, the second incubation involves culturing cells derived from the cell aggregate (e.g. spheroid) in a “maturation media.” In some embodiments, the second incubation involves culturing cells derived from the cell aggregate (e.g. spheroid) in the basal induction media, and then in the maturation media.

In some embodiments, the second incubation involves culturing the cells in the basal induction media from about day 7 through about day 10. In some embodiments, the second incubation involves comprises culturing the cells in the maturation media beginning on about day 11. In some embodiments, the second incubation involves culturing the cells in the basal induction media from about day 7 through about day 10, and then in the maturation media beginning on about day 11. In some embodiments, cells are cultured in the maturation media to produce determined dopaminergic cells or dopaminergic neurons.

In some embodiments, the basal induction media is formulated to contain Neurobasal™ media and DMEM/F12 media at a 1:1 ratio, supplemented with N-2 and B27 supplements, non-essential amino acids (NEAA), GlutaMAX™, L-glutamine, β-mercaptoethanol, and insulin.

In some embodiments, the maturation media is formulated to contain Neurobasal™ media, supplemented with N-2 and B27 supplements, non-essential amino acids (NEAA), and GlutaMAX™.

In some embodiments, the cells are cultured in the basal induction media from about day 7 up to about day 11 (e.g., day 10 or day 11). In some embodiments, the cells are cultured in the basal induction media from about day 7 through day 10, each day inclusive. In some embodiments, the cells are cultured in the maturation media beginning on about day 11. In some embodiments, the cells are cultured in the basal induction media from about day 7 through about day 10, and then the cells are cultured in the maturation media beginning on about day 11. In some embodiments, the cells are cultured in the maturation media from about day 11 until harvest or collection of the cells. In some embodiments, cells are harvested between day 16 and 27. In some embodiments, cells are harvested between day 18 and day 25. In some embodiments, cells are harvested on day 18. In some embodiments, cells are harvested on day 25.

In some embodiments, the test cells are from an in vitro population from a culture of cells differentiated from pluripotent cells that are subjected to a differentiation protocol for inducing the differentiation of PSCs, e.g., iPSCs, into dopaminergic neurons, such as according to any of the methods described herein.

In some embodiments, cells of the second differentiation state are in any of days 15-21 of the differentiation protocol. In some embodiments, cells of the first differentiation state are at day 14 or earlier, day 13 or earlier, day 12 or earlier, or day 11 or earlier of the differentiation protocol. In some embodiments, cells of the third differentiation state are at day 22 or later, day 23 or later, day 24 or later, or day 25 or later of the differentiation protocol. In some embodiments, cells of the first differentiation state are at day 14 or earlier, day 13 or earlier, day 12 or earlier, or day 11 or earlier of the differentiation protocol; cells of the second differentiation state are in any of days 15-21 of the differentiation protocol; and cells of the third differentiation state are at day 22 or later, day 23 or later, day 24 or later, or day 25 or later of the differentiation protocol. In some embodiments, cells of the first differentiation state are in any of days 10-14 of the differentiation protocol. In some embodiments, cells of the third differentiation state are at day 25 or later of the differentiation protocol.

In some embodiments, cells of the second differentiation state are in any of days 16-18 of the differentiation protocol. In some embodiments, cells of the first differentiation state are at day 15 or earlier, day 14 or earlier, day 13 or earlier, day 12 or earlier, or day 11 or earlier of the differentiation protocol. In some embodiments, cells of the third differentiation state are at day 19 or later, day 20 or later, day 11 or later, day 22 or later, day 23 or later, day 24 or later, or day 25 or later of the differentiation protocol. In some embodiments, cells of the first differentiation state are at day 15 or earlier, day 14 or earlier, day 13 or earlier, day 12 or earlier, or day 11 or earlier of the differentiation protocol; cells of the second differentiation state are in any of days 16-18 of the differentiation protocol; and cells of the third differentiation state are at day 19 or later, day 20 or later, day 21 or later, day 22 or later, day 23 or later, day 24 or later, or day 25 or later of the differentiation protocol. In some embodiments, cells of the first differentiation state are in any of days 11-13 of the differentiation protocol. In some embodiments, cells of the third differentiation state are at day 25 or later of the differentiation protocol.

In some embodiments, cells of the second differentiation state are in any of days 17-19 of the differentiation protocol. In some embodiments, cells of the first differentiation state are at day 16 or earlier, day 15 or earlier, day 14 or earlier, day 13 or earlier, day 12 or earlier, or day 11 or earlier of the differentiation protocol. In some embodiments, cells of the third differentiation state are at day 20 or later, day 11 or later, day 22 or later, day 23 or later, day 24 or later, or day 25 or later of the differentiation protocol. In some embodiments, cells of the first differentiation state are at day 16 or earlier, day 15 or earlier, day 14 or earlier, day 13 or earlier, day 12 or earlier, or day 11 or earlier of the differentiation protocol; cells of the second differentiation state are in any of days 17-19 of the differentiation protocol; and cells of the third differentiation state are at day 20 or later, day 21 or later, day 22 or later, day 23 or later, day 24 or later, or day 25 or later of the differentiation protocol. In some embodiments, cells of the first differentiation state are in any of days 12-14 of the differentiation protocol. In some embodiments, cells of the third differentiation state are at day 25 or later of the differentiation protocol.

In some embodiments, cells of the second differentiation state are in any of days 15-17 of the differentiation protocol. In some embodiments, cells of the first differentiation state are at day 14 or earlier, day 13 or earlier, day 12 or earlier, or day 11 or earlier of the differentiation protocol. In some embodiments, cells of the third differentiation state are at day 18 or later, day 19 or later, day 20 or later, day 11 or later, day 22 or later, day 23 or later, day 24 or later, or day 25 or later of the differentiation protocol. In some embodiments, cells of the first differentiation state are at day 14 or earlier, day 13 or earlier, day 12 or earlier, or day 11 or earlier of the differentiation protocol; cells of the second differentiation state are in any of days 15-17 of the differentiation protocol; and cells of the third differentiation state are at day 18 or later, day 19 or later, day 20 or later, day 21 or later, day 22 or later, day 23 or later, day 24 or later, or day 25 or later of the differentiation protocol. In some embodiments, cells of the first differentiation state are in any of days 10-12 of the differentiation protocol. In some embodiments, cells of the third differentiation state are at day 30 or later of the differentiation protocol.

4. Pluripotent Stem Cells

In some embodiments, the test cells and/or reference cell populations are produced from pluripotent stem cells. Various sources of pluripotent stem cells can be used, including embryonic stem (ES) cells and induced pluripotent stem cells (iPSCs). In some embodiments, the pluripotent stem cells are iPSCs. iPSCs may be generated by a process known as reprogramming, wherein non-pluripotent cells are effectively “dedifferentiated” to an embryonic stem cell-like state by engineering them to express genes such as OCT4, SOX2, and KLF4 (Takahashi and Yamanaka Cell (2006) 126: 663-76). In some embodiments, the pluripotent stem cells are iPSCs that were artificially derived from non-pluripotent cells of a subject. In some embodiments, the non-pluripotent cells are fibroblasts. In some embodiments, the subject is a human. In some embodiments, the subject is a human with Parkinson's Disease.

In some aspects, pluripotency refers to cells with the ability to give rise to progeny that can undergo differentiation, under appropriate conditions, into cell types that collectively exhibit characteristics associated with cell lineages from the three germ layers (endoderm, mesoderm, and ectoderm). Pluripotent stem cells can contribute to tissues of a prenatal, postnatal, or adult organism. A standard art-accepted test, such as the ability to form a teratoma in 8-12 week old SCID mice, can be used to establish the pluripotency of a cell population. However, identification of various pluripotent stem cell characteristics can also be used to identify pluripotent cells. In some aspects, pluripotent stem cells can be distinguished from other cells by particular characteristics, including by expression or non-expression of certain combinations of molecular markers. More specifically, human pluripotent stem cells may express at least some, and optionally all, of the markers from the following non-limiting list: SSEA-3, SSEA-4, TRA-1-60, TRA-1-81, TRA-2-49/6E, ALP, Sox2, E-cadherin, UTF-1, Oct4, Lin28, Rex1, and Nanog. In some aspects, a pluripotent stem cell characteristic is a cell morphology associated with pluripotent stem cells.

Methods for generating iPSCs are known. For example, mouse iPSCs were reported in 2006 (Takahashi and Yamanaka), and human iPSCs were reported in late 2007 (Takahashi et al. and Yu et al.). Mouse iPSCs demonstrate important characteristics of pluripotent stem cells, including the expression of stem cell markers, the formation of tumors containing cells from all three germ layers, and the ability to contribute to many different tissues when injected into mouse embryos at a very early stage in development. Human iPSCs also express stem cell markers and are capable of generating cells characteristic of all three germ layers.

In some embodiments, non-pluripotent cells (e.g., fibroblasts) derived from patients having Parkinson's disease (PD) are reprogrammed to become iPSCs before differentiation into neuronal cells. In some embodiments, fibroblasts may be reprogrammed to iPSCs by transforming fibroblasts with genes (OCT4, SOX2, NANOG, LIN28, and KLF4) cloned into a plasmid (for example, see, Yu, et al., Science DOI: 10.1126/science.1172482). In some embodiments, non-pluripotent fibroblasts derived from patients having PD are reprogrammed to become differentiation into determined dopaminergic cells and/or dopaminergic neurons, such as by use of the non-integrating Sendai virus to reprogram the cells (e.g., use of CTS™ CytoTune™-iPS 2.1 Sendai Reprogramming Kit). In some embodiments, the resulting differentiated cells are then administered to the patient from whom they are derived in an autologous stem cell transplant. In some embodiments, the PSCs (e.g., iPSCs) are allogeneic to the subject to be treated, i.e., the PSCs are derived from a different individual than the subject to whom the differentiated cells will be administered. In some embodiments, non-pluripotent cells (e.g., fibroblasts) derived from another individual (e.g., an individual not having a neurodegenerative disorder, such as Parkinson's disease) are reprogrammed to become iPSCs before differentiation into determined dopaminergic cells and/or dopaminergic neurons. In some embodiments, reprogramming is accomplished, at least in part, by use of the non-integrating Sendai virus to reprogram the cells (e.g., use of CTS™ CytoTune™_iPS 2.1 Sendai Reprogramming Kit). In some embodiments, the resulting differentiated cells are then administered to an individual who is not the same individual from whom the differentiated cells are derived (e.g. allogeneic cell therapy or allogeneic cell transplantation).

In any of the provided embodiments, the PSCs described herein may be genetically engineered to be hypoimmunogenic. Methods for reducing the immunogenicity are known and include ablating polymorphic HLA-A/-B/-C and HLA class II molecule expression and introducing the immunomodulatory factors PD-L1, HLA-G, and CD47 into the AAVS1 safe harbor locus in differentiated cells (Han et al., PNAS (2019) 116(21):10441-46). Thus, in some embodiments, the PSCs described herein are engineered to delete highly polymorphic HLA-A/-B/-C genes and to introduce immunomodulatory factors, such as PD-L1, HLA-G, and/or CD47, into the AAVS1 safe harbor locus.

In some embodiments, PSCs (e.g., iPSCs) are cultured in the absence of feeder cells until they reach 80-90% confluency, at which point they are harvested and further cultured for differentiation (day 0). In some aspects, once iPSCs reach 80-90% confluence, they are washed in phosphate buffered saline (PBS) and subjected to enzymatic dissociation, such as with Accutase™, until the cells are easily dislodged from the surface of a culture vessel. The dissociated iPSCs are then re-suspended in media for downstream differentiation into the desired cell type(s), such as determined dopaminergic cells and/or dopaminergic neurons.

In some embodiments, the PSCs are resuspended in a basal induction media. In some embodiments, the basal induction media is formulated to contain Neurobasal™ media and DMEM/F12 media at a 1:1 ratio, supplemented with N-2 and B27 supplements, non-essential amino acids (NEAA), GlutaMAX™, L-glutamine, β-mercaptoethanol, and insulin. In some embodiments, the basal induction media is further supplemented with serum replacement, a Rho-associated protein kinase (ROCK) inhibitor, and various small molecules for differentiation. In some embodiments, the PSCs are resuspended in the same media they will be cultured in for at least a portion of the first incubation.

5. Exemplary Characteristics of Classified Cells

In some embodiments, cells of the in vitro population of cells identified as having the desired differentiation state, e.g., the second differentiation state, are able to survive when administered in vivo, e.g., to an animal model. In some embodiments, cells of the identified in vitro population survive following transplantation into an animal or human subject. In some embodiments, cells of the identified in vitro population of cells have therapeutic effect to treat a disease or condition in an animal model. In some embodiments, cells of the identified in vitro population of cells have therapeutic effect to treat a disease or condition in human patients. In some embodiments, the cells when implanted ameliorate or reverse symptoms of the disease or condition.

In some embodiments, cells of the in vitro population of cells identified as having the desired differentiation state, e.g., the second differentiation state, which can be that of determined dopaminergic neuronal cells, express a marker of a midbrain dopaminergic neuron, such as FOXA2 or tyrosine hydroxylase (TH). In some embodiments, the cells express TH (TH+). In some embodiments, the cells express FOXA2 (FOXA2+). In some embodiments, the cells express TH and FOXA2 (TH+FOXA2+).

In some embodiments, cells of the identified in vitro population of cells are determined to or capable of becoming dopaminergic neurons, i.e., are determined dopaminergic cells, as ascertained based on one or more characteristics that indicate the cells are capable of having functional activity of a dopaminergic neuron but may not yet express a marker of a dopaminergic neuron or may not express it at a high level. For example, the cells may exhibit lower levels of TH than a dopaminergic neuron, yet still exhibit one or more characteristics of a determined dopaminergic cell indicating the cells are capable of having functional activity of a dopaminergic neuron. In some embodiments, the one or more characteristics include activity to survive, engraft, and/or innervate other cells when administered in vivo, e.g., to an animal model. In some embodiments, cells of the identified in vitro population are capable of innervating host tissue following transplantation into an animal or human subject. In some embodiments, cells of the identified in vitro population exhibit neurite outgrowth following transplantation into an animal or human subject. In some embodiments, cells of the identified in vitro population survive following transplantation into an animal or human subject. In some embodiments, cells of the identified in vitro population engraft following transplantation into an animal or human subject.

In some embodiments, cells of the identified in vitro population of cells have therapeutic effect to treat a neurodegenerative disease in an animal model of a neurodegenerative disease. In some embodiments, the neurodegenerative disease is Parkinson's disease. Any suitable animal model of Parkinson's disease can be used for screening. In some embodiments, the animal model is a lesion model wherein animals received unilateral stereotaxic injection of 6-hydroxydopamine (6-OHDA) into the substantia nigra. In some embodiments, the animal model is a lesion model wherein animals received unilateral stereotaxic injection of 6-OHDA into the medial forebrain bundle. In some embodiments, the cells are implanted into the substantia nigra of the animal model. In some embodiments, a behavioral assay is performed to screen for therapeutic effects of the implantation on the animal model. In some embodiments, the behavioral assay comprises monitoring amphetamine-induced circling behavior. In some embodiments, the cells reduce, decrease or reverse a Parkinsonian model brain lesion in this model.

In some embodiments, cells of the identified in vitro population of cells have therapeutic effect to treat a neurodegenerative disease, including in human patients. In some embodiments, the cells when implanted ameliorate or reverse symptoms of a neurodegenerative disease. In some embodiments, the neurodegenerative disease is Parkinson's disease. In some embodiments, the cells when implanted in the substantia nigra of a subject, e.g., patient, in need thereof improve Parkinsonian symptoms.

D. Gene Expression Levels

In some embodiments, the gene expression levels, e.g., of any of the test cells or reference cell populations described herein, are determined based on the levels of a gene product synthesized using information encoded by a gene or genes. In some embodiments, a gene product is any biomolecule that is assembled, generated, and/or synthesized with information encoded by a gene, and may include polynucleotides and/or polypeptides. In some embodiments, assessing, measuring, and/or determining gene expression includes determining or measuring the level, amount, or concentration of the gene product. In some embodiments, the level, amount, or concentration of the gene product may be transformed (e.g., normalized) or directly analyzed (e.g., raw).

In some embodiments, the gene product includes a protein, i.e., a polypeptide, that is encoded by and/or expressed by the gene. In particular embodiments, the gene product encodes a protein that is localized and/or exposed on the surface of a cell. In some embodiments, the protein is a soluble protein. In certain embodiments, the protein is secreted by a cell. In particular embodiments, the gene expression is the amount, level, and/or concentration of a protein that is encoded by the gene. In certain embodiments, one or more protein gene products are measured by any suitable means. Suitable methods for assessing, measuring, determining, and/or quantifying the level, amount, or concentration of one or more protein gene products include detection with immunoassays, nucleic acid-based or protein-based aptamer techniques, HPLC (high precision liquid chromatography), peptide sequencing (such as Edman degradation sequencing or mass spectrometry (such as MS/MS), optionally coupled to HPLC), and microarray adaptations of any of the foregoing (including nucleic acid, antibody, or protein-protein (i.e., non-antibody) arrays). In some embodiments, the immunoassay is or includes methods or assays that detect proteins based on an immunological reaction, e.g., by detecting the binding of an antibody or antigen binding antibody fragment to a gene product. Immunoassays include quantitative immunocytochemisty or immunohistochemisty, ELISA (including direct, indirect, sandwich, competitive, multiple, and portable ELISAs (see, e.g., U.S. Pat. No. 7,510,687), western blotting (including one, two, or higher dimensional blotting or other chromatographic means, optionally including peptide sequencing), enzyme immunoassay (EIA), RIA (radioimmunoassay), and SPR (surface plasmon resonance).

In certain embodiments, the gene product is a polynucleotide, e.g., an mRNA, or a protein that is encoded by the gene. In some embodiments, the gene product is a polynucleotide that is expressed by and/or encoded by the gene. In certain embodiments, the polynucleotide is an RNA. In some embodiments, the gene product is a messenger RNA (mRNA), a transfer RNA (tRNA), a ribosomal RNA, a small nuclear RNA, a small nucleolar RNA, an antisense RNA, long non-coding RNA, a microRNA, a Piwi-interacting RNA, a small interfering RNA, and/or a short hairpin RNA. In particular embodiments, the gene product is an mRNA.

In particular embodiments, assessing, measuring, determining, and/or quantifying the amount or level of an RNA gene product includes a step of generating, polymerizing, and/or deriving a cDNA polynucleotide and/or a cDNA oligonucleotide from the RNA gene product. In certain embodiments, the RNA gene product is assessed, measured, determined, and/or quantified by directly assessing, measuring, determining, and/or quantifying a cDNA polynucleotide and/or a cDNA oligonucleotide that is derived from the RNA gene product.

In particular embodiments, the amount or level of a polynucleotide in a sample may be assessed, measured, determined, and/or quantified by any suitable means. For example, in some embodiments, the amount or level of a polynucleotide gene product can be assessed, measured, determined, and/or quantified by polymerase chain reaction (PCR), including reverse transcriptase (rt) PCR, droplet digital PCR, and real-time and quantitative PCR (qPCR) methods (including, e.g., TAQMAN®, molecular beacon, LIGHTUP™, SCORPION™ SIMPLEPROBES®; see, e.g., U.S. Pat. Nos. 5,538,848; 5,925,517; 6,174,670; 6,329,144; 6,326,145, and 6,635,427); northern blotting; Southern blotting, e.g., of reverse transcription products and derivatives; array based methods, including blotted arrays, microarrays, or in situ-synthesized arrays; and sequencing, e.g., sequencing by synthesis, pyrosequencing, dideoxy sequencing, or sequencing by ligation, or other methods such as discussed in Shendure et al., Nat. Rev. Genet. 5:335-44 (2004) or Nowrousian, Euk. Cell 9(9): 1300-1310 (2010), including such specific platforms as HELICOS®, ROCHE® 454, ILLUMINA®/SOLEXA®, ABI SOLiD®, and POLONATOR® sequencing. In particular embodiments, the levels of nucleic acid gene products are measured by quantitative PCR (qPCR) methods, such qRT-PCR. In some embodiments, the qRT-PCR uses three nucleic acid sets for each gene, where the three nucleic acids comprise a primer pair together with a probe that binds between the regions of a target nucleic acid where the primers bind—known commercially as a TAQMAN® assay.

In particular embodiments, the expression of two or more of the genes are measured or assessed simultaneously. In certain embodiments, a multiplex PCR, e.g., a multiplex rt-PCR assessing or a multiplex quantitative PCR (qPCR), is used for measuring, determining, and/or quantifying the level, amount, or concentration of two or more gene products. In some embodiments, microarrays (e.g., AFFYMETRIX®, AGILENT®, and ILLUMINA®-style arrays) are used for assessing, measuring, determining, and/or quantifying the level, amount, or concentration of two or more gene products. In some embodiments, microarrays are used for assessing, measuring, determining, and/or quantifying the level, amount, or concentration of a cDNA polynucleotide that is derived from an RNA gene product. In some embodiments, the expression of one or more gene products, e.g., polynucleotide gene products, is determined by sequencing the gene product and/or by sequencing a cDNA polynucleotide that is derived from the from the gene product. In some embodiments, the sequencing is performed by a non-Sanger sequencing method and/or a next generation sequencing (NGS) technique. Examples of Next Generation Sequencing techniques include Massively Parallel Signature Sequencing (MPSS), Polony sequencing, pyrosequencing, Reversible dye-terminator sequencing, SOLiD sequencing, Ion semiconductor sequencing, DNA nanoball sequencing, Helioscope single molecule sequencing, Single molecule real time (SMRT) sequencing, Single molecule real time (RNAP) sequencing, and Nanopore DNA sequencing.

In some embodiments, the NGS technique is RNA sequencing (RNA-Seq). In particular embodiments, the expression of the one or more polynucleotide gene products is measured, determined, and/or quantified by RNA-Seq. RNA-Seq, also called whole transcriptome shotgun sequencing, determines the presence and quantity of RNA in a sample. RNA sequencing methods have been adapted for the most common DNA sequencing platforms [such as HiSeq systems (Illumina), 454 Genome Sequencer FLX System (Roche), Applied Biosystems SOLiD (Life Technologies), and IonTorrent (Life Technologies)]. These platforms require initial reverse transcription of RNA into cDNA. Conversely, the single molecule sequencer HeliScope (Helicos BioSciences) is able to use RNA as a template for sequencing. A proof of principle for direct RNA sequencing on the PacBio RS platform has also been demonstrated (Pacific Bioscience). In some embodiments, the one or more RNA gene products are assessed, measured, determined, and/or quantified by RNA-seq. In some embodiments, the RNA-seq is a tag-based RNA-seq. In tag-based methods, each transcript is represented by a unique tag. Initially, tag-based approaches were developed as a sequence-based method to measure transcript abundance and identify differentially expressed genes, assuming that the number of tags (counts) directly corresponds to the abundance of the mRNA molecules. The reduced complexity of the sample, obtained by sequencing a defined region, was essential to making the Sanger-based methods affordable. When NGS technology became available, the high number of reads that could be generated facilitated differential gene expression analysis. A transcript length bias in the quantification of gene expression levels, such as observed for shotgun methods, is not encountered in tag-based methods. All tag-based methods are by definition strand specific. In particular embodiments, the one or more RNA gene products are assessed, measured, determined, and/or quantified by tag-based RNA-seq.

In some embodiments, the RNA-seq is a shotgun RNA-seq. Numerous protocols have been described for shotgun RNA-seq, but they have many steps in common: fragmentation (which can occur at the RNA level or cDNA level, conversion of the RNA into cDNA (performed by oligo dT or random primers), second-strand synthesis, ligation of adapter sequences at the 3′ and 5′ ends (at RNA or DNA level) and final amplification. In some embodiments, RNA-seq can focus only on polyadenylated RNA molecules (mainly mRNAs but also some lncRNAs, snoRNAs, pseudogenes, and histones) if poly(A)+RNAs are selected prior to fragmentation, or may also include non-polyadenylated RNAs if no selection is performed. In the latter case, ribosomal RNA (more than 80% of the total RNA pool) needs to be depleted prior to fragmentation. It is, therefore, clear that differences in capturing of the mRNA part of the transcriptome lead to a partial overlap in the type of detected transcripts. Moreover, different protocols may affect the abundance and the distribution of the sequenced reads. This makes it difficult to compare results from experiments with different library preparation protocols.

In some embodiments, RNA from each sample is obtained, fragmented, and used to generate complementary DNA (cDNA) samples, such as cDNA libraries for sequencing. Reads may be processed and aligned to the human genome, and the expected number of mappings per gene/isoform are estimated and used to determine read counts. In some embodiments, read counts are normalized by the length of the genes/isoforms and number of reads in a library to yield FPKM normalized, e.g., by length of the genes/isoforms and number of reads in the library, to yield fragments per kilobase of exon per million mapped reads (FPKM) according to the gene length and total mapped reads. In some aspects, between—sample normalization is achieved by normalization, such as 75th quantile normalization, where each sample is scaled by the median of 75th quantiles from all samples, e.g., to yield quantile-normalized FPKM (FPKQ) values. The FPKQ values may be log-transformed (log 2).

In some embodiments, RNA from each sample is obtained, fragmented, and used to generate complementary DNA (cDNA) samples, such as cDNA libraries for sequencing. Reads may be processed and aligned to the human genome, and the expected number of mappings per gene/isoform are estimated and used to determine read counts. In some embodiments, read counts are normalized by the length of the genes/isoforms and number of reads in a library. In some embodiments, read counts are provided as counts per million (CPM). In some embodiments, the CPM read counts are log-transformed (e.g., log₂).

In some embodiments, relative gene expression is measured by comparing the CPM of a target gene to the CPM of a housekeeping gene. In some embodiments, the housekeeping gene is GAPDH. In some embodiments, the relative gene expression of a target gene is determined as the ratio of the CPM of the target gene to CPM of a housekeeping gene (e.g. GAPDH).

In some embodiments, the gene expression levels are obtained using microarray analysis. In some embodiments, the gene expression levels are obtained using RNA sequencing. In some embodiments, the gene expression levels are obtained using both microarray analysis and RNA sequencing. In some embodiments, the RNA sequencing is performed on bulk RNA from a plurality of cells. In some embodiments, bulk RNA sequencing data is obtained from pooled RNA from the plurality of cells. In some embodiments, the RNA sequencing is performed on single cells. In some embodiments, the RNA sequencing is performed on bulk RNA from a plurality of cells and on single cells.

Any suitable methods for obtaining bulk RNA sequencing data can be used (for example, see Chao et al., 2019, BMC Genomics 20: 571, incorporated by reference herein in its entirety). For instance, total RNA from a sample, e.g., a plurality of cells from a population of cells, can be isolated using TRIZOL, treated with DNase I, and purified. Concentration and quality of isolated RNA can be measured and checked prior to library preparation for total RNA or mRNA. For library preparation, total RNA or mRNA can be fragmented and converted to cDNA using reverse transcription. After construction, amplification, and optional barcoding of double-stranded cDNA, libraries can be processed for next generation sequencing using any suitable library preparation techniques, sequencing platforms, and genomic-alignment tools.

In some embodiments, the gene expression levels are obtained using single-cell RNA sequencing. In some embodiments, the use of single-cell RNA sequencing data affords certain advantages. In some embodiments, the use of single-cell RNA sequencing data allows for characterization of subpopulations of cells, for instance of determined dopaminergic cells within a larger population of cells. In some embodiments, the use of single-cell RNA sequencing data reduces the number of cells required for use in the methods provided herein, e.g., reduces the number of cells needed to obtain data for training a machine learning model. In some embodiments, the use of single-cell RNA sequencing data improves characteriziation of biological variability across cells. In some embodiments, the use of single-cell RNA sequencing data allows for easier validation and interpretation of gene expression levels.

Any suitable methods for single-cell RNA sequencing can be used (for example, see Zheng et al., 2017 (Nature Communications 8: 14049), and Haque et al., 2017 (Genome Medicine 9: 75, incorporated by reference herein in their entirety). For single-RNA sequencing, single cells from a sample, for instance an in vitro population of cells, can be isolated using flow cytometric cell-sorting, microfluidic platform, or droplet-based methods. Isolated cells are lysed to allow capture of RNA molecules. Poly[T]-primers can be used for the analysis of polyadenylated mRNA molecules specifically, and primed mRNA molecules are converted to cDNA using reverse transcription. In some instances, unique molecular identifiers can be used to mark single mRNA molecules based on cellular origin. The cDNA pool can then amplified, optionally barcoded, and sequenced, for instance using next-generation sequencing (NGS) and with library preparation techniques, sequencing platforms, and genomic-alignment tools similar to those used for bulk RNA samples. In some instances, unbiased cell-type classification witin a mixed population of distinct cell types can be achieved with as few as 10,000 to 50,000 reads per cell, and single-cell libraries from various common protocols can be close to saturation when sequenced to a depth of 1,000,000 reads.

In some embodiments, the gene expression levels include bulk RNA sequencing data and single-cell RNA sequencing data. In some embodiments, the bulk RNA sequencing data and the single-cell RNA sequencing data are obtained from the same population of cells. In some embodiments, the single-cell RNA sequencing data can be used to approximate the bulk RNA sequencing data obtained from the same population of cells. In some embodiments, approximated bulk RNA sequencing data is obtained by averaging single-cell RNA sequencing data from cells in the same population of cells. In some embodiments, the gene expression levels include approximated bulk RNA sequencing data.

### III. COMPUTING DEVICES

Also provided herein in some embodiments are computing devices for classifying the differentiation state of an in vitro population of cells. In some embodiments, the provided computing devices are for identifying an in vitro population of cells having a desired differentiation state.

In some embodiments, the computing device includes a memory that includes a first reference dataset and a second reference dataset. Exemplary first and second reference datasets are described in Section II-A. In some embodiments, the first and second reference datasets are any as described in Section II-A.

In some embodiments, the memory further includes one or more additional reference datasets. In some embodiments, the one or more additional reference datasets include any of the first and second reference datasets described in Section II-A.

In some embodiments, the memory further includes a control dataset. Exemplary control datasets are described in Section II-A. In some embodiments, the control dataset is any as described in Section II-A.

In some embodiments, the computing device includes instructions stored in memory for performing any of the provided methods. In some embodiments, the computing device further includes a processor that implements the instructions stored in memory. In some embodiments, the processor includes one or more processing elements in communication with a system data store (SDS) comprising one or more storage elements. In some embodiments, the processor includes one or more processing elements, such as a CELERON, PENTIUM, XEON, CORE 2 DUO, or CORE 2 QUAD class microprocessor (Intel Corp., Santa Clara, Calif.), or SEMPRON, PHENOM, OPTERON, ATHLON X2, or ATHLON 64 X2 (AMD Corp., Sunnyvale. Calif.), although other general purpose processors could be used. In some embodiments, the functionality may be distributed across multiple processing elements. The term processing element may refer to (1) a process running on a particular piece, or across particular pieces, of hardware, (2) a particular piece of hardware, or either (1) or (2) as the context allows. Some implementations can include one or more limited special purpose processors, such as a digital signal processor (DSP), application specific integrated circuits (ASIC), or a field programmable gate arrays (FPGA). Further, some implementations can use combinations of general purpose and special purpose processors.

In some embodiments, the computing device includes one or more input devices for receiving input from users and/or software applications. In some embodiments, the input includes a test dataset. Exemplary test datasets are described in Section II-A. In some embodiments, the test dataset is any as described in Section II-A.

In some embodiments, the computing device includes one or more output devices for presenting output to users and/or software applications. In some embodiments, the output devices present an output of any of the provided methods. In some embodiments, the output devices include a monitor capable of displaying to a user graphical representation of the output.

In some embodiments, the computing device further includes a SDS that could include a variety of primary and secondary storage elements. In one implementation, the SDS would include registers and RAM as part of the primary storage. The primary storage may in some implementations include other forms of memory such as cache memory or non-volatile memory (e.g., FLASH, ROM, or EPROM). The SDS may also include secondary storage including single, multiple, and/or varied servers and storage elements. For example, the SDS may use internal storage devices connected to the system processor. In implementations where a single processing element supports all of the functionality, a local hard disk drive may serve as the secondary storage of the SDS, and a disk operating system executing on such a single processing element may act as a data server receiving and servicing data requests.

It will be understood by those skilled in the art that the different information used in the systems and methods as disclosed herein may be logically or physically segregated within a single device serving as secondary storage for the SDS; multiple related data stores accessible through a unified management system, which together serve as the SDS; or multiple independent data stores individually accessible through disparate management systems, which may in some implementations be collectively viewed as the SDS. The various storage elements that comprise the physical architecture of the SDS may be centrally located or distributed across a variety of diverse locations.

In addition, or instead, the functionality and approaches discussed above, or portions thereof, can be embodied in instructions executable by a computer, where such instructions are stored in and/or on one or more computer readable storage media. Such media can include primary storage and/or secondary storage integrated with and/or within the computer such as RAM and/or a magnetic disk, and/or separable from the computer such as on a solid state device or removable magnetic or optical disk. The media can use any technology, including ROM, RAM, magnetic, optical, paper, and/or solid state media technology. In some embodiments, the computing device can be a multipurpose machine having modules and/or components dedicated to the performance of the disclosed methods.

### IV. COMPOSITIONS AND FORMULATIONS

Provided herein in some embodiments are pharmaceutical compositions containing populations of cells, including populations of cells, e.g., stem-cell derived cells, identified by any of the provided methods as having a desired differentiation state, such as any of the methods described in Section II.

In some embodiments, the cells in the provided therapeutic compositions include stem-cell derived neuronal cells. In some embodiments, the stem-cell derived neuronal cells are suitable for treatment of a neurodegenerative disease when implanted into a brain of a subject in need of such treatment. In some embodiments, the cells in the provided therapeutic compositions include determined dopaminergic (DA) neuronal cells. In some embodiments, the cells in the provided therapeutic compositions are stem-cell derived neuronal cells that are capable of engrafting in a brain region following implantation.

In some embodiments, the cells in the composition are an in vitro stem cell-derived neuronal cell population. In some embodiments, the in vitro stem cell-derived neuronal cell population is characterized by cells that express one or more genes selected from the group consisting of CCNB2, AURKB, PTTG1, TOP2A, NEUROG2, HES1, REST, E2F4, FOXM1, SIN3A, NFYA, LIN28A, FLRT3, ITGA5, NES, SOX2, SOX9 and RFX4. In some embodiments, the cells in the population are characterized by expressing of only one of the above genes. In some embodiments, the cells in the population are characterized by expression 2, 3, 4, 5, 6, 7, 8, 9, 10, 11, 12, 13, 14, 15, 16, 17, or 18 of the above genes. In some embodiments, at least one of the one or more genes is REST.

In some embodiments, at least 50% of cells within the in vitro stem-cell derived neuronal cell population express the one or more genes. In some embodiments, at least 60% of cells within the in vitro stem-cell derived neuronal cell population express the one or more genes. In some embodiments, at least 70% of cells within the in vitro stem-cell derived neuronal cell population express the one or more genes. In some embodiments, at least 80% of cells within the in vitro stem-cell derived neuronal cell population express the one or more genes. In some embodiments, at least 90% of cells within the in vitro stem-cell derived neuronal cell population express the one or more genes.

In some aspects, the expression of the one or more genes is RNA expression. In some embodiments, the RNA expression is measured by RNA sequencing. In other aspects, the expression of the one or more genes is protein expression.

In some embodiments, the population of cells in the provided composition has been differentiated in vitro from a pluripotent stem cell (PSC). The differentiation may be carried out by any of the methods as described in Section C. In particular embodiments, the methods involve differentiating iPSCs into neuronal progenitor cells including for producing determined dopaminergic neurons.

In some of any embodiments, the one or more genes is a gene that is overexpressed in cells of the population compared to the iPSCs. In some embodiments, the one or more genes is a gene that is overexpressed in cells of the population compared to cells of a precursor population differentiated from the iPSCs. For instance, in some embodiments, the one or more gene is a gene that is overexpressed compared to cells of a precursor population of cells at a differentiation stage before the cells are, or are likely suspected, of being determined dopaminergic neurons. For example, the precursor population of cells may be day 13 cells of a dopaminergic differentiation protocol as described herein. In some embodiments, the one or more genes is a gene that is overexpressed in cells of the population compared to cells of a mature committed dopaminergic neuronal cell population differentiated from the iPSCs. For instance, in some embodiments, the one or more gene is a gene that is overexpressed compared to a mature or committed population of cells at a differentiation stage before the cells are, or are likely suspected, of being determined dopaminergic neurons. For example, the mature commited cells may be day 25 cells of a dopaminergic differentiation protocol as described herein. In some embodiments, the mature committed dopaminergic neuronal cells express LMX1A and/or NR4A2 (NURR1). In some embodiments, among cells in the committed dopaminergic neuronal cell population, at least 40%, at least 50%, at least 60%, at least 70%, or at least 80% of the cells express LMX1A and/or NR4A2. In some embodiments, the overexpression is a positive log 2 fold change of greater than or greater than about 1.5-fold, 2.0-fold, 3.0-fold, 4.0-fold or 5-fold.

In some of any embodiments of the in vitro stem-cell derived neuronal cell population, the one or more genes is a gene that is reduced in expression in cells of the population compared to the iPSCs. In some embodiments, one or more gene is a gene that is reduced in expression in cells of the population compared to cells of a precursor population differentiated from the iPSCs. For instance, in some embodiments, the one or more gene is a gene that is reduced in expression compared to cells of a precursor population of cells at a differentiation stage before the cells are, or are likely suspected, of being determined dopaminergic neurons. For example, the precursor population of cells may be day 13 cells of a dopaminergic differentiation protocol as described herein. In some embodiments, the one or more genes is a gene that is reduced in expression in cells of the population compared to cells of a mature committed dopaminergic neuronal cell population differentiated from the iPSCs. For instance, in some embodiments, the one or more gene is a gene that is reduced in expression compared to a mature or committed population of cells at a differentiation stage before the cells are, or are likely suspected, of being determined dopaminergic neurons. For example, the mature commited cells may be day 25 cells of a dopaminergic differentiation protocol as described herein. In some embodiments, the mature committed dopaminergic neuronal cells express LMX1A and/or NR4A2 (NURR1). In some embodiments, among cells in the committed dopaminergic neuronal cell population, at least 40%, at least 50%, at least 60%, at least 70%, or at least 80% of the cells express LMX1A and/or NR4A2. In some embodiments, the reduced expression is a negative log 2 fold change of greater than or greater than about 1.5-fold, 2.0-fold, 3.0-fold, 4.0-fold or 5-fold.

In some of any embodiments of the in vitro stem-cell derived neuronal cell population, less than 30%, less than 20%, or less than 10% of the cells in the population express LMX1A and/or NR4A2.

In some of any embodiments of the in vitro stem-cell derived neuronal cell population, cells in the population are capable of engrafting in and innervating other cells in vivo. In some embodiments, cells in the population are capable of exhibiting neurite outgrowth when administered to the brain of a subject. In some embodiments, cells in the population are capable of producing dopamine. In some embodiments, cells in the population do not produce or do not substantially produce norepinephrine.

In some embodiments, the cells in the provided therapeutic compositions are capable of producing dopamine (DA). In some embodiments, the cells in the provided therapeutic compositions do not produce or do not substantially produce norepinephrine (NE). Thus, in some embodiments, the cells in the provided therapeutic compositions are capable of producing DA, but do not produce or do not substantially produce NE.

In some embodiments, the determined DA neuronal cells express EN1. In some embodiments, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80% of the total cells in the therapeutic composition express EN1. In some embodiments, at least about 20% of the cells of the therapeutic composition express EN1. In some embodiments, at least about 25% of the cells of the therapeutic composition express EN1. In some embodiments, at least about 30% of the cells of the therapeutic composition express EN1. In some embodiments, at least about 35% of the cells of the therapeutic composition express EN1. In some embodiments, at least about 40% of the cells of the therapeutic composition express EN1. In some embodiments, at least about 45% of the cells of the therapeutic composition express EN1. In some embodiments, at least about 50% of the cells of the therapeutic composition express EN1. In some embodiments, at least about 55% of the cells of the therapeutic composition express EN1. In some embodiments, at least about 60% of the cells of the therapeutic composition express EN1. In some embodiments, at least about 65% of the cells of the therapeutic composition express EN1. In some embodiments, at least about 70% of the cells of the therapeutic composition express EN1. In some embodiments, at least about 75% of the cells of the therapeutic composition express EN1. In some embodiments, at least about 80% of the cells of the therapeutic composition express EN1.

In some embodiments, the therapeutic composition exhibits a ratio of counts per million (CPM) EN1 to CPM GAPDH of greater than about 1×10⁻⁴. In some embodiments, the ratio of CPM EN1 to CPM GAPDH is between about 1.5×10⁻³ and 1×10⁻².

In some embodiments, the determined DA neuronal cells express CORIN. In some embodiments, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80% of the total cells in the therapeutic composition express CORIN. In some embodiments, at least about 20% of the cells of the therapeutic composition express CORIN. In some embodiments, at least about 25% of the cells of the therapeutic composition express CORIN. In some embodiments, at least about 30% of the cells of the therapeutic composition express CORIN. In some embodiments, at least about 35% of the cells of the therapeutic composition express CORIN. In some embodiments, at least about 40% of the cells of the therapeutic composition express CORIN. In some embodiments, at least about 45% of the cells of the therapeutic composition express CORIN. In some embodiments, at least about 50% of the cells of the therapeutic composition express CORIN. In some embodiments, at least about 55% of the cells of the therapeutic composition express CORIN. In some embodiments, at least about 60% of the cells of the therapeutic composition express CORIN. In some embodiments, at least about 65% of the cells of the therapeutic composition express CORIN. In some embodiments, at least about 70% of the cells of the therapeutic composition express CORIN. In some embodiments, at least about 75% of the cells of the therapeutic composition express CORIN. In some embodiments, at least about 80% of the cells of the therapeutic composition express CORIN.

In some embodiments, the therapeutic composition exhibits a ratio of counts per million (CPM) CORIN to CPM GAPDH of greater than about 1×10⁻⁴. In some embodiments, the ratio of CPM CORIN to CPM GAPDH is between about 5×10⁻² and 5×10⁻¹.

In In some embodiments, the determined DA neuronal cells express EN1 and CORIN. In some embodiments, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80% of the total cells in the therapeutic composition express EN1 and CORIN. In some embodiments, at least about 20% of the cells of the therapeutic composition express EN1 and CORIN. In some embodiments, at least about 25% of the cells of the therapeutic composition express EN1 and CORIN. In some embodiments, at least about 30% of the cells of the therapeutic composition express EN1 and CORIN. In some embodiments, at least about 35% of the cells of the therapeutic composition express EN1 and CORIN. In some embodiments, at least about 40% of the cells of the therapeutic composition express EN1 and CORIN. In some embodiments, at least about 45% of the cells of the therapeutic composition express EN1 and CORIN. In some embodiments, at least about 50% of the cells of the therapeutic composition express EN1 and CORIN. In some embodiments, at least about 55% of the cells of the therapeutic composition express EN1 and CORIN. In some embodiments, at least about 60% of the cells of the therapeutic composition express EN1 and CORIN. In some embodiments, at least about 65% of the cells of the therapeutic composition express EN1 and CORIN. In some embodiments, at least about 70% of the cells of the therapeutic composition express EN1 and CORIN. In some embodiments, at least about 75% of the cells of the therapeutic composition express EN1 and CORIN. In some embodiments, at least about 80% of the cells of the therapeutic composition express EN1 and CORIN.

In some embodiments, the therapeutic composition exhibits (a) a ratio of counts per million (CPM) EN1 to CPM GAPDH of greater than about 1×10⁻⁴; and (b) a ratio of CPM CORIN to CPM GAPDH of greater than about 2×10⁻². In some embodiments, the ratio of CPM EN1 to CPM GAPDH is between about 1.5×10⁻³ and 1×10⁻²; and the ratio of CPM CORIN to CPM GAPDH of between about 5×10⁻² and 5×10⁻¹.

In some embodiments, less than 10% of the determined DA neuronal cells express TH. In some embodiments, the determined DA neuronal cells express low levels of TH. In some embodiments, the determined DA neuronal cells do not express TH. In some embodiments, the determined DA neuronal cells express TH at lower levels than cells harvested or collected on other days. In some embodiments, some of the determined DA neuronal cells express EN1 and CORIN and less than 10% of the determined DA neuronal cells express TH. In some embodiments, less than 8% of the determined DA neuronal cells express TH. In some embodiments, less than 5% of the determined DA neuronal cells express TH.

In some embodiments, between about 2% and 10%, between about 2% and 8%, between about 2% and 6%, between about 2% and 4%, between about 4% and 10%, between about 4% and 8%, between about 4% and 6%, between about 6% and 10%, between about 6% and 8%, or between about 8% and 10% of the total cells in the therapeutic composition express TH.

In some embodiments, the therapeutic composition exhibits a ratio of counts per million (CPM) TH to CPM GAPDH of less than about 3×10⁻². In some embodiments, the ratio of CPM TH to CPM GAPDH is between about 1×10⁻³ and 2.5×10⁻².

In some embodiments, less than 10% of the total cells in the therapeutic composition express TH, and at least about 20% of the cells of the therapeutic composition express EN1. In some embodiments, less than 10% of the total cells in the therapeutic composition express TH, and at least about 25% of the cells of the therapeutic composition express EN1. In some embodiments, less than 10% of the total cells in the therapeutic composition express TH, and at least about 30% of the cells of the therapeutic composition express EN1. In some embodiments, less than 10% of the total cells in the therapeutic composition express TH, and at least about 35% of the cells of the therapeutic composition express EN1. In some embodiments, less than 10% of the total cells in the therapeutic composition express TH, and at least about 40% of the cells of the therapeutic composition express EN1. In some embodiments, less than 10% of the total cells in the therapeutic composition express TH, and at least about 45% of the cells of the therapeutic composition express EN1. In some embodiments, less than 10% of the total cells in the therapeutic composition express TH, and at least about 50% of the cells of the therapeutic composition express EN1. In some embodiments, less than 10% of the total cells in the therapeutic composition express TH, and at least about 55% of the cells of the therapeutic composition express EN1. In some embodiments, less than 10% of the total cells in the therapeutic composition express TH, and at least about 60% of the cells of the therapeutic composition express EN1. In some embodiments, less than 10% of the total cells in the therapeutic composition express TH, and at least about 65% of the cells of the therapeutic composition express EN1. In some embodiments, less than 10% of the total cells in the therapeutic composition express TH, and at least about 70% of the cells of the therapeutic composition express EN1. In some embodiments, less than 10% of the total cells in the therapeutic composition express TH, and at least about 75% of the cells of the therapeutic composition express EN1. In some embodiments, less than 10% of the total cells in the therapeutic composition express TH, and at least about 80% of the cells of the therapeutic composition express EN1.

In some embodiments, less than 10% of the total cells in the therapeutic composition express TH, and at least about 20% of the cells of the therapeutic composition express CORIN. In some embodiments, less than 10% of the total cells in the therapeutic composition express TH, and at least about 25% of the cells of the therapeutic composition express CORIN. In some embodiments, less than 10% of the total cells in the therapeutic composition express TH, and at least about 30% of the cells of the therapeutic composition express CORIN. In some embodiments, less than 10% of the total cells in the therapeutic composition express TH, and at least about 35% of the cells of the therapeutic composition express CORIN. In some embodiments, less than 10% of the total cells in the therapeutic composition express TH, and at least about 40% of the cells of the therapeutic composition express CORIN. In some embodiments, less than 10% of the total cells in the therapeutic composition express TH, and at least about 45% of the cells of the therapeutic composition express CORIN. In some embodiments, less than 10% of the total cells in the therapeutic composition express TH, and at least about 50% of the cells of the therapeutic composition express CORIN. In some embodiments, less than 10% of the total cells in the therapeutic composition express TH, and at least about 55% of the cells of the therapeutic composition express CORIN. In some embodiments, less than 10% of the total cells in the therapeutic composition express TH, and at least about 60% of the cells of the therapeutic composition express CORIN. In some embodiments, less than 10% of the total cells in the therapeutic composition express TH, and at least about 65% of the cells of the therapeutic composition express CORIN. In some embodiments, less than 10% of the total cells in the therapeutic composition express TH, and at least about 70% of the cells of the therapeutic composition express CORIN. In some embodiments, less than 10% of the total cells in the therapeutic composition express TH, and at least about 75% of the cells of the therapeutic composition express CORIN. In some embodiments, less than 10% of the total cells in the therapeutic composition express TH, and at least about 80% of the cells of the therapeutic composition express CORIN.

In some embodiments, less than 10% of the total cells in the therapeutic composition express TH, and at least about 20% of the cells of the therapeutic composition express EN1 and CORIN. In some embodiments, less than 10% of the total cells in the therapeutic composition express TH, and at least about 25% of the cells of the therapeutic composition express EN1 and CORIN. In some embodiments, less than 10% of the total cells in the therapeutic composition express TH, and at least about 30% of the cells of the therapeutic composition express EN1 and CORIN. In some embodiments, less than 10% of the total cells in the therapeutic composition express TH, and at least about 35% of the cells of the therapeutic composition express EN1 and CORIN. In some embodiments, less than 10% of the total cells in the therapeutic composition express TH, and at least about 40% of the cells of the therapeutic composition express EN1 and CORIN. In some embodiments, less than 10% of the total cells in the therapeutic composition express TH, and at least about 45% of the cells of the therapeutic composition express EN1 and CORIN. In some embodiments, less than 10% of the total cells in the therapeutic composition express TH, and at least about 50% of the cells of the therapeutic composition express EN1 and CORIN. In some embodiments, less than 10% of the total cells in the therapeutic composition express TH, and at least about 55% of the cells of the therapeutic composition express EN1 and CORIN. In some embodiments, less than 10% of the total cells in the therapeutic composition express TH, and at least about 60% of the cells of the therapeutic composition express EN1 and CORIN. In some embodiments, less than 10% of the total cells in the therapeutic composition express TH, and at least about 65% of the cells of the therapeutic composition express EN1 and CORIN. In some embodiments, less than 10% of the total cells in the therapeutic composition express TH, and at least about 70% of the cells of the therapeutic composition express EN1 and CORIN. In some embodiments, less than 10% of the total cells in the therapeutic composition express TH, and at least about 75% of the cells of the therapeutic composition express EN1 and CORIN. In some embodiments, less than 10% of the total cells in the therapeutic composition express TH, and at least about 80% of the cells of the therapeutic composition express EN1 and CORIN.

In some embodiments, the provided therapeutic compositions are pharmaceutical compositions containing a pharmaceutically acceptable carrier. In some embodiments, the dose of cells including cells classified by any of the methods disclosed herein is provided as a composition or formulation, such as a pharmaceutical composition or formulation. Such compositions can be used in accord with the provided methods, articles of manufacture, and/or with the provided compositions, such as in the prevention or treatment of diseases, conditions, and disorders, such as neurodegenerative disorders.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes a buffer, excipient, stabilizer, or preservative.

In some aspects, the choice of carrier is determined in part by the particular cell or agent and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the therapeutic compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Any suitable methods for preparing administrable pharmaceutical compositions can be used. Exemplary methods are described in more detail in, for example, Remington: The Science and Practice of Pharmacy, Lippincott Williams & Wilkins; 21st ed. (May 1, 2005).

The formulation or composition may also contain more than one active ingredient useful for the particular indication, disease, or condition being prevented or treated with the cells or agents, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as carbidopa-levodopa (e.g., Levodopa), dopamine agonists (e.g., pramipexole, ropinirole, rotigotine, and apomorphine), MAO B inhibitors (e.g., selegiline, rasagiline, and safinamide), catechol O-methyltransferase (COMT) inhibitors (e.g., entacapone and tolcapone), anticholinergics (e.g., benztropine and trihexylphenidyl), amantadine. In some embodiments, the agents or cells are administered in the form of a salt, e.g., a pharmaceutically acceptable salt. Suitable pharmaceutically acceptable acid addition salts include those derived from mineral acids, such as hydrochloric, hydrobromic, phosphoric, metaphosphoric, nitric, and sulphuric acids, and organic acids, such as tartaric, acetic, citric, malic, lactic, fumaric, benzoic, glycolic, gluconic, succinic, and arylsulphonic acids, for example, p-toluenesulphonic acid.

The formulation or composition may also be administered in combination with another form of treatment useful for the particular indication, disease, or condition being prevented or treated with the cells or agents, where the respective activities do not adversely affect one another. Thus, in some embodiments, the pharmaceutical composition is administered in combination with deep brain stimulation (DBS).

The pharmaceutical composition in some embodiments contains agents or cells in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the therapeutic composition, by multiple bolus administrations of the therapeutic composition, or by continuous infusion administration of the therapeutic composition.

The agents or cells can be administered by any suitable means, for example, by stereotactic injection (e.g., using a catheter). In some embodiments, a given dose is administered by a single bolus administration of the cells or agent. In some embodiments, it is administered by multiple bolus administrations of the cells or agent, for example, over a period of months or years. In some embodiments, the agents or cells can be administered by stereotactic injection into the brain, such as in the striatum.

For the prevention or treatment of disease, the appropriate dosage may depend on the type of disease to be treated, the type of agent or agents, the type of cells or recombinant receptors, the severity and course of the disease, whether the agent or cells are administered for preventive or therapeutic purposes, previous therapy, the subject's clinical history and response to the agent or the cells, and the discretion of the attending physician. The therapeutic compositions are in some embodiments suitably administered to the subject at one time or over a series of treatments.

The cells or agents may be administered using standard administration techniques, formulations, and/or devices. Provided are formulations and devices, such as syringes and vials, for storage and administration of the therapeutic compositions. With respect to cells, administration can be autologous. For example, non-pluripotent cells (e.g., fibroblasts) can be obtained from a subject, and administered to the same subject following reprogramming and differentiation. When administering a therapeutic composition (e.g., a pharmaceutical composition containing a genetically reprogrammed and/or differentiated cell or an agent that treats or ameliorates symptoms of a disease or disorder, such as a neurodegenerative disorder), it will generally be formulated in a unit dosage injectable form (solution, suspension, emulsion). Formulations include those for stereotactic administration, such as into the brain (e.g. the striatum).

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the agent or cells in a solvent, such as in admixture with a suitable carrier, diluent, or excipient such as sterile water, physiological saline, glucose, dextrose, or the like.

The formulations to be used for in vivo administration are generally sterile. Sterility may be readily accomplished, e.g., by filtration through sterile filtration membranes.

### V. ARTICLES OF MANUFACTURE AND KITS

Also provided herein in some embodiments are articles of manufacture that include any of the provided therapeutic compositions. Also provided herein in some embodiments are kits including (i) any of the provided therapeutic compositions and (ii) instructions for administering the therapeutic composition to a subject.

In some embodiments, the articles of manufacture or kits include one or more containers, typically a plurality of containers, packaging material, and a label or package insert on or associated with the container or containers and/or packaging. In some embodiments, the instructions provide directions or specify methods for assessing if a subject, prior to receiving a cell therapy, is likely or suspected of being likely to respond and/or the degree or level of response following administration of cells for treating a disease or disorder. In some aspects, the articles of manufacture can contain a dose or a composition of differentiated cells.

The articles of manufacture provided herein contain packaging materials. Packaging materials for use in packaging the provided materials are well known to those of skill in the art. See, for example, U.S. Pat. Nos. 5,323,907, 5,052,558, and 5,033,252, each of which is incorporated herein in its entirety. Examples of packaging materials include, but are not limited to, blister packs, bottles, tubes, inhalers, pumps, bags, vials, containers, syringes, disposable laboratory supplies, e.g., pipette tips and/or plastic plates, or bottles. The articles of manufacture or kits can include a device so as to facilitate dispensing of the materials or to facilitate use in a high-throughput or large-scale manner, e.g., to facilitate use in robotic equipment. Typically, the packaging is non-reactive with the therapeutic compositions contained therein.

In some embodiments, the compositions are packaged separately. In some embodiments, each container can have a single compartment. In some embodiments, other components of the articles of manufacture or kits are packaged separately or together in a single compartment.

### VI. METHODS OF TREATMENT

Provided herein in some embodiments are methods of using any of the provided therapeutic compositions for treating a disease or condition in a subject in need thereof. In some embodiments, the provided methods include implanting a population of cells having a desired differentiation state into a subject. In some embodiments, the population of cells is one that is identified as having the desired differentiation state according to any of the provided methods. In some embodiments, the provided methods include selecting a population of stem-cell derived neuronal cells having a desired differentiation state using any of the provided methods, and implanting the selected population of neuronal cells into the subject. In some embodiments, the stem-cell derived neuronal cells having the desired differentiation state are determined dopaminergic neuronal cells, and the population of cells is implanted into a brain region of the subject.

Such methods and uses include therapeutic methods and uses, for example, involving administration of the therapeutic cells, or compositions containing the same, to a subject having a disease, condition, or disorder. In some embodiments, the disease or condition is a neurodegenerative disease or condition. In some embodiments, the cells or pharmaceutical composition thereof is administered in an effective amount to effect treatment of the disease or disorder. Uses include uses of the cells or pharmaceutical compositions thereof in such methods and treatments, and in the preparation of a medicament in order to carry out such therapeutic methods. In some embodiments, the methods thereby treat the disease or condition or disorder in the subject.

In some embodiments, a subject has a neurodegenerative disease. In some embodiments, the neurodegenerative disease comprises the loss of dopamine neurons in the brain. In some embodiments, the subject has lost dopamine neurons in the substantia nigra (SN). In some embodiments, the subject has lost dopamine neurons in the substantia nigra pas compacta (SNc). In some embodiments, the subject exhibits rigidity, bradykinesia, postural reflect impairment, resting tremor, or a combination thereof. In some embodiments, the subject exhibits abnormal [18F]-L-DOPA PET scan. In some embodiments, the subject exhibits [18F]-DG-PET evidence for a Parkinson's Disease Related Pattern (PDRP).

In some embodiments, the neurodegenerative disease is Parkinsonism. In some embodiments, the neurodegenerative disease is Parkinson's disease. Parkinson's disease (PD) is the second most common neurodegenerative, estimated to affect 4-5 million patients worldwide. This number is predicted to more than double by 2030. PD is the second most common neurodegenerative disorder after Alzheimer's disease, affecting approximately 1 million patients in the US with 60,000 new patients diagnosed each year. Currently there is no cure for PD, which is characterized pathologically by a selective loss of midbrain DA neurons in the substantia nigra. A fundamental characteristic of PD is therefore progressive, severe and irreversible loss of midbrain dopamine (DA) neurons resulting in ultimately disabling motor dysfunction. In some aspects, the methods, compositions, and uses thereof provided herein contemplate administration of differentiated cells, e.g., determined DA neuronal progentiro cells, to subjects exhibiting a loss of dopamine (DA) neurons, including Parkinson's disease.

In some embodiments, the neurodegenerative disease is idiopathic Parkinson's disease. In some embodiments, the neurodegenerative disease is a familial form of Parkinson's disease. In some embodiments, the subject has mild Parkinson's disease. In some embodiments, the subject has a Movement Disorder Society-Unified Parkinson's Disease Rating Scale (MDS-UPDRS) motor score of less than or equal to 32. In some embodiments, the subject has moderate or advanced Parkinson's disease. In some embodiments, the subject has mild Parkinson's disease. In some embodiments, the subject has a MDS-UPDRS motor score of between 33 and 60.

In some embodiments, a dose of cells is administered to subjects in accord with the provided methods, and/or with the provided articles of manufacture or compositions. In some embodiments, the size or timing of the doses is determined as a function of the particular disease or condition in the subject. In some cases, the size or timing of the doses for a particular disease in view of the provided description may be empirically determined.

In some embodiments, the dose of cells is administered to the striatum of the subject. In some embodiments, the dose of cells is administered to one hemisphere of the subject's striatum. In some embodiments, the dose of cells is administered to both hemispheres of the subject's.

In some embodiments, the dose of cells administered to the subject is about 5×10⁶ cells. In some embodiments, the dose of cells administered to the subject is about 10×10⁶ cells. In some embodiments, the dose of cells administered to the subject is about 15×10⁶ cells. In some embodiments, the dose of cells administered to the subject is about 20×10⁶ cells. In some embodiments, the dose of cells administered to the subject is about 25×10⁶ cells. In some embodiments, the dose of cells administered to the subject is about 30×10⁶ cells.

In some embodiments, the dose of cells comprises between at or about 250,000 cells per hemisphere and at or about 20 million cells per hemisphere, between at or about 500,000 cells per hemisphere and at or about 20 million cells per hemisphere, between at or about 1 million cells per hemisphere and at or about 20 million cells per hemisphere, between at or about 5 million cells per hemisphere and at or about 20 million cells per hemisphere, between at or about 10 million cells per hemisphere and at or about 20 million cells per hemisphere, between at or about 15 million cells per hemisphere and at or about 20 million cells per hemisphere, between at or about 250,000 cells per hemisphere and at or about 15 million cells per hemisphere, between at or about 500,000 cells per hemisphere and at or about 15 million cells per hemisphere, between at or about 1 million cells per hemisphere and at or about 15 million cells per hemisphere, between at or about 5 million cells per hemisphere and at or about 15 million cells per hemisphere, between at or about 10 million cells per hemisphere and at or about 15 million cells per hemisphere, between at or about 250,000 cells per hemisphere and at or about 10 million cells per hemisphere, between at or about 500,000 cells per hemisphere and at or about 10 million cells per hemisphere, between at or about 1 million cells per hemisphere and at or about 10 million cells per hemisphere, between at or about 5 million cells per hemisphere and at or about 10 million cells per hemisphere, between at or about 250,000 cells per hemisphere and at or about 5 million cells per hemisphere, between at or about 500,000 cells per hemisphere and at or about 5 million cells per hemisphere, between at or about 1 million cells per hemisphere and at or about 5 million cells per hemisphere, between at or about 250,000 cells per hemisphere and at or about 1 million cells per hemisphere, between at or about 500,000 cells per hemisphere and at or about 1 million cells per hemisphere, or between at or about 250,000 cells per hemisphere and at or about 500,00 cells per hemisphere.

In some embodiments, the dose of cells is between at or about 1 million cells per hemisphere and at or about 30 million cells per hemisphere. In some embodiments, the dose of cells is between at or about 5 million cells per hemisphere and at or about 20 million cells per hemisphere. In some embodiments, the dose of cells is between at or about 10 million cells per hemisphere and at or about 15 million cells per hemisphere.

In some embodiments, the dose of cells is between about about 3×10⁶ cells/hemisphere and 15×10⁶ cells/hemisphere. In some embodiments, the dose of cells is about about 3×10⁶ cells/hemisphere. In some embodiments, the dose of cells is about about 4×10⁶ cells/hemisphere. In some embodiments, the dose of cells is about about 5×10⁶ cells/hemisphere. In some embodiments, the dose of cells is about about 6×10⁶ cells/hemisphere. In some embodiments, the dose of cells is about about 7×10⁶ cells/hemisphere. In some embodiments, the dose of cells is about about 8×10⁶ cells/hemisphere. In some embodiments, the dose of cells is about about 9×10⁶ cells/hemisphere. In some embodiments, the dose of cells is about about 10×10⁶ cells/hemisphere. In some embodiments, the dose of cells is about about 11×10⁶ cells/hemisphere. In some embodiments, the dose of cells is about about 12×10⁶ cells/hemisphere. In some embodiments, the dose of cells is about about 13×10⁶ cells/hemisphere. In some embodiments, the dose of cells is about about 14×10⁶ cells/hemisphere. In some embodiments, the dose of cells is about about 15×10⁶ cells/hemisphere.

In some embodiments, the number of cells administered to the subject is between about 0.25×10⁶ total cells and about 20×10⁶ total cells, between about 0.25×10⁶ total cells and about 15×10⁶ total cells, between about 0.25×10⁶ total cells and about 10×10⁶ total cells, between about 0.25×10⁶ total cells and about 5×10⁶ total cells, between about 0.25×10⁶ total cells and about 1×10⁶ total cells, between about 0.25×10⁶ total cells and about 0.75×10⁶ total cells, between about 0.25×10⁶ total cells and about 0.5×10⁶ total cells, between about 0.5×10⁶ total cells and about 20×10⁶ total cells, between about 0.5×10⁶ total cells and about 15×10⁶ total cells, between about 0.5×10⁶ total cells and about 10×10⁶ total cells, between about 0.5×10⁶ total cells and about 5×10⁶ total cells, between about 0.5×10⁶ total cells and about 1×10⁶ total cells, between about 0.5×10⁶ total cells and about 0.75×10⁶ total cells, between about 0.75×10⁶ total cells and about 20×10⁶ total cells, between about 0.75×10⁶ total cells and about 15×10⁶ total cells, between about 0.75×10⁶ total cells and about 10×10⁶ total cells, between about 0.75×10⁶ total cells and about 5×10⁶ total cells, between about 0.75×10⁶ total cells and about 1×10⁶ total cells, between about 1×10⁶ total cells and about 20×10⁶ total cells, between about 1×10⁶ total cells and about 15×10⁶ total cells, between about 1×10⁶ total cells and about 10×10⁶ total cells, between about 1×10⁶ total cells and about 5×10⁶ total cells, between about 5×10⁶ total cells and about 20×10⁶ total cells, between about 5×10⁶ total cells and about 15×10⁶ total cells, between about 5×10⁶ total cells and about 10×10⁶ total cells, between about 10×10⁶ total cells and about 20×10⁶ total cells, between about 10×10⁶ total cells and about 15×10⁶ total cells, or between about 15×10⁶ total cells and about 20×10⁶ total cells.

In certain embodiments, the cells, or individual populations of sub-types of cells, are administered to the subject at a range of about 5 million cells per hemisphere to about 20 million cells per hemisphere or any value in between these ranges. Dosages may vary depending on attributes particular to the disease or disorder and/or patient and/or other treatments.

In some embodiments, the patient is administered multiple doses, and each of the doses or the total dose can be within any of the foregoing values. In some embodiments, the dose of cells comprises the administration of from or from about 5 million cells per hemisphere to about 20 million cells per hemisphere, each inclusive.

In some embodiments, the dose of cells, e.g. differentiated cells, is administered to the subject as a single dose or is administered only one time within a period of two weeks, one month, three months, six months, 1 year or more.

In the context of stem cell transplant, administration of a given “dose” encompasses administration of the given amount or number of cells as a single composition and/or single uninterrupted administration, e.g., as a single injection or continuous infusion, and also encompasses administration of the given amount or number of cells as a split dose or as a plurality of compositions, provided in multiple individual compositions or infusions, over a specified period of time, such as a day. Thus, in some contexts, the dose is a single or continuous administration of the specified number of cells, given or initiated at a single point in time. In some contexts, however, the dose is administered in multiple injections or infusions in a single period, such as by multiple infusions over a single day period.

Thus, in some aspects, the cells of the dose are administered in a single pharmaceutical composition. In some embodiments, the cells of the dose are administered in a plurality of compositions, collectively containing the cells of the dose.

In some embodiments, cells of the dose may be administered by administration of a plurality of compositions or solutions, such as a first and a second, optionally more, each containing some cells of the dose. In some aspects, the plurality of compositions, each containing a different population and/or sub-types of cells, are administered separately or independently, optionally within a certain period of time.

In some embodiments, the administration of the composition or dose, e.g., administration of the plurality of cell compositions, involves administration of the cell compositions separately. In some aspects, the separate administrations are carried out simultaneously, or sequentially, in any order.

In some embodiments, the subject receives multiple doses, e.g., two or more doses or multiple consecutive doses, of the cells. In some embodiments, two doses are administered to a subject. In some embodiments, multiple consecutive doses are administered following the first dose, such that an additional dose or doses are administered following administration of the consecutive dose. In some aspects, the number of cells administered to the subject in the additional dose is the same as or similar to the first dose and/or consecutive dose. In some embodiments, the additional dose or doses are larger than prior doses.

In some aspects, the size of the first and/or consecutive dose is determined based on one or more criteria such as response of the subject to prior treatment, e.g. disease stage and/or likelihood or incidence of the subject developing adverse outcomes, e.g., dyskinesia.

In some embodiments, the dose of cells is generally large enough to be effective in improving symptoms of the disease.

In some embodiments, the cells are administered at a desired dosage, which in some aspects includes a desired dose or number of cells or cell type(s) and/or a desired ratio of cell types. In some embodiments, the dosage of cells is based on a desired total number (or number per kg of body weight) of cells in the individual populations or of individual cell types (e.g., TH+ or TH−). In some embodiments, the dosage is based on a combination of such features, such as a desired number of total cells, desired ratio, and desired total number of cells in the individual populations.

Thus, in some embodiments, the dosage is based on a desired fixed dose of total cells and a desired ratio, and/or based on a desired fixed dose of one or more, e.g., each, of the individual sub-types or sub-populations.

In particular embodiments, the numbers and/or concentrations of cells refer to the number of TH-negative cells. In other embodiments, the numbers and/or concentrations of cells refer to the number or concentration of all cells administered.

In some embodiments, the cells are administered at a desired dosage, which in some aspects includes a desired dose or number of cells or cell type(s) and/or a desired ratio of cell types. Thus, the dosage of cells in some embodiments is based on a total number of cells and a desired ratio of the individual populations or sub-types In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

Thus, in some embodiments, the dosage is based on a desired fixed dose of total cells and a desired ratio, and/or based on a desired fixed dose of one or more, e.g., each, of the individual sub-types or sub-populations.

In particular embodiments, the numbers and/or concentrations of cells refer to the number of TH-negative cells. In other embodiments, the numbers and/or concentrations of cells refer to the number or concentration of all cells administered.

In some aspects, the size of the dose is determined based on one or more criteria such as response of the subject to prior treatment, e.g. disease type and/or stage, and/or likelihood or incidence of the subject developing toxic outcomes, e.g., dyskinesia.

### VII. EXEMPLARY EMBODIMENTS

Among the provided embodiments are:

1. A computing device for classifying the differentiation state of an in vitro population of cells, the device comprising a memory that comprises:
a first reference dataset that comprises a representation of gene expression levels for one or more genes that are differentially expressed between cells at a first differentiation state and cells at a second differentiation state; and
a second reference dataset that comprises a representation of gene expression levels for one or more genes that are differentially expressed between cells at the second differentiation state and cells at a third differentiation state.

2. The computing device of embodiment 1, further comprising a processor that implements instructions stored in the memory to perform a method comprising:
(a) receiving as input a test dataset that comprises expression levels for genes that are expressed in one or more test cells comprised in an in vitro population of cells, wherein the expression levels in the test dataset comprise expression levels for (i) one or more of the genes for which a representation of expression levels are included in the first reference dataset, and (ii) one or more of the genes for which a representation of expression levels are included in the second reference dataset;
(b) calculating, using the test dataset and the first reference dataset, a first similarity score indicating whether the differentiation state of the test cells is more similar to the first differentiation state or to the second differentiation state;
(c) calculating, using the test dataset and the second reference dataset, a second similarity score indicating whether the differentiation state of the test cells is more similar to the second differentiation state or to the third differentiation state; and
(d) classifying the differentiation state of the one or more test cells based on the first similarity score and the second similarity score.

3. The computing device of embodiment 1 or embodiment 2, wherein the memory further comprises a control dataset that comprises a representation of gene expression levels for one or more genes that are expressed in cells at one or more control differentiation states, which control differentiation state may be the same as or different than one of the first, second or third differentiation states.

4. The computing device of embodiment 3, wherein:
the test dataset comprises gene expression levels for one or more of the genes for which a representation of expression levels are included in the control dataset;
the instructions comprise calculating a degree of correlation between the representation of gene expression levels for one or more genes in the control dataset and gene expression levels for the one or more genes in the test dataset to calculate a correlation score; and
the classifying the differentiation state of the one or more test cells is based on the first similarity score, the second similarity score, and the correlation score.

5. The computing device of embodiment 4, wherein the correlation score is calculated prior to calculating the first similarity score and the second similarity score, and the method is terminated if the correlation score for the test cells does not meet a predefined cutoff value.

6. The computing device of any of embodiments 3-5, wherein the control dataset comprises gene expression levels that are normalized by counts per million mapped reads (CPM) and filtered to include only gene expression levels that exceed a threshold CPM value.

7. The computing device of any of embodiments 3-6, wherein the control dataset comprises a centroid of gene expression levels of the one or more genes in the control dataset.

8. The computing device of embodiment 7, wherein the correlation score is calculated by normalizing the gene expression levels of the one or more genes in the test dataset and calculating a correlation of the gene expression levels of the one or more genes in the test dataset to the centroid.

9. The computing device of embodiment 8, wherein the control dataset comprises coefficient of variation (CV) values of gene expression levels of the one or more genes in the control dataset, and the correlation to the centroid is weighted by the inverse of the CV values.

10. The computing device of any of embodiments 1-9, wherein the in vitro population of cells is from a culture of cells differentiated from pluripotent cells that are subjected to suitable differentiation conditions.

11. The computing device of any of embodiments 1-10, wherein the first differentiation state is earlier in a stem cell differentiation pathway than the second differentiation state.

12. The computing device of any of embodiments 1-11, wherein the second differentiation state is earlier in a stem cell differentiation pathway than the third differentiation state.

13. The computing device of any of embodiments 1-9, wherein the first differentiation state is in a cell differentiation pathway that is parallel to a cell differentiation pathway of the second differentiation state.

14. The computing device of any of embodiments 1-13, wherein the population of cells are selected from the group consisting of stem-cell derived cardiac muscle cells, stem-cell derived skeletal muscle cells, stem-cell derived kidney tubule cells, stem-cell derived red blood cell cells, stem-cell derived smooth muscle cells, stem-cell derived lung cells, stem-cell derived thyroid cells, stem-cell derived pancreatic cells, stem-cell derived epidermal cells, stem-cell derived pigment cells, and stem-cell derived neuronal cells.

15. The computing device of any of embodiments 1-14, wherein the population of cells are stem-cell derived neuronal cells.

16. The computing device of any of embodiments 1-15, wherein the second differentiation state is the differentiation state of a determined dopaminergic neuronal cell.

17. The computing device of any of embodiments 1-16, wherein the second differentiation state is the differentiation state of cells with fitness for engraftment.

18. The computing device of any of embodiments 1-17, wherein the first reference dataset comprises a representation of gene expression levels for one or more genes selected from Table E1.

19. The computing device of any of embodiments 1-18, wherein the second reference dataset comprises a representation of gene expression levels for one or more genes selected from Table E2.

20. The computing device of any of embodiments 1-19, wherein the first reference dataset comprises a representation of gene expression levels for at least 20 genes selected from Table E1.

21. The computing device of any of embodiments 1-20, wherein the second reference dataset comprises a representation of gene expression levels for at least 20 genes selected from Table E2.

22. The computing device of any of embodiments 1-21, wherein the first reference dataset comprises a representation of gene expression levels for at least 50 genes selected from Table E1.

23. The computing device of any of embodiments 1-22, wherein the second reference dataset comprises a representation of gene expression levels for at least 50 genes selected from Table E2.

24. The computing device of any of embodimentsm 1-23, wherein at least one of the first, second and third differentiation states is characterized using an in vitro assay.

25. The computing device of any of embodiments 1-24, wherein at least one of the first, second and third differentiation states is characterized using an in vivo assay.

26. The computing device of embodiment 25, wherein the in vivo assay comprises determining whether reference cells are capable of surviving, engrafting, and/or innervating tissue when administered to an animal or human subject.

27. The computing device of embodiment 25 or embodiment 26, wherein the in vivo assay comprises determining whether reference cells ameliorate or reverse symptoms of a neurodegenerative disease when implanted into an animal or human subject.

28. The computing device of embodiment 26 or embodiment 27, wherein the animal subject comprises an animal model of Parkinson's disease.

29. The computing device of any of embodiments 1-28, wherein the memory further comprises one or more additional reference datasets, wherein each of the additional reference datasets comprises a representation of gene expression levels for one or more genes that are differentially expressed between cells at the second differentiation state and cells at an additional differentiation state, wherein:
the processor implements instructions to calculate, using the additional reference datasets, one or more additional similarity scores indicating whether the differentiation state of the test cells is more similar to the second differentiation state or to one of the one or more additional differentiation states, and
the classifying the differentiation state of the one or more test cells is based on the first similarity score, the second similarity score, and the one or more additional similarity scores.

30. The computing device of any of embodiments 1-29, wherein the representations of gene expression levels in the first reference dataset and/or the second reference dataset are obtained using machine learning.

31. The computing device of embodiment 30, wherein the machine learning comprises principal component analysis.

32. The computing device of any of embodiments 1-29, wherein the representations of gene expression levels in the first reference dataset and/or the second reference dataset comprise normalized gene expression levels.

33. The computing device of any of embodiments 1-32, wherein the differentiation state of the one or more test cells is classified as being the second differentiation state if the first and second similarity scores indicate that the differentiation state of the one or more test cells is more similar to the second differentiation state.

34. A method for selecting a population of cells having a desired differentiation state, the method comprising:
(a) calculating a first similarity score using a test dataset and a first reference dataset, wherein:
the first reference dataset comprises a representation of gene expression levels for one or more genes that are differentially expressed between cells at a first differentiation state and cells at a second differentiation state,
the test dataset comprises expression levels for genes that are expressed in one or more test cells comprised in an in vitro population of cells, wherein the expression levels in the test dataset comprise expression levels for one or more of the genes for which a representation of expression levels are included in the first reference dataset, and
the first similarity score indicates whether the differentiation state of the test cells is more similar to the first differentiation state or to the second differentiation state;
(b) calculating a second similarity score using the test dataset and a second reference dataset, wherein:
the second reference dataset comprises a representation of gene expression levels for one or more genes that are differentially expressed between cells at the second differentiation state and cells at a third differentiation state,
the expression levels in the test dataset comprise expression levels for one or more of the genes for which a representation of expression levels are included in the second reference dataset, and
the second similarity score indicates whether the differentiation state of the test cells is more similar to the second differentiation state or to the third differentiation state; and
(c) classifying the differentiation state of the one or more test cells based on the first similarity score and the second similarity score.

35. The method of embodiment 34, wherein:
the test dataset comprises gene expression levels for one or more genes for which a representation of expression levels are included in a control dataset that comprises a representation of gene expression levels for one or more genes that are expressed in cells at a control differentiation state, which control differentiation state may be the same as or different than one of the first, second or third differentiation states;
the method further comprises calculating a degree of correlation between the representation of gene expression levels for one or more genes in the control dataset and gene expression levels for the one or more genes in the test dataset to calculate a correlation score; and
the classifying the differentiation state of the one or more test cells is based on the first similarity score, the second similarity score, and the correlation score.

36. The method of embodiment 35, wherein the correlation score is calculated prior to calculating the first similarity score and the second similarity score and the method is terminated if the correlation score for the test cells does not meet a predefined cutoff value.

37. The method of embodiment 35 or embodiment 36, wherein the control dataset comprises gene expression levels that are normalized by counts per million mapped reads (CPM) and filtered to include only gene expression levels that exceed a threshold CPM value.

38. The method of any of embodiments 35-37, wherein the control dataset comprises a centroid of gene expression levels of the one or more genes in the control dataset.

39. The method of embodiment 38, wherein the correlation score is calculated by normalizing the gene expression levels of the one or more genes in the test dataset and calculating a correlation of the gene expression levels of the one or more genes in the test dataset to the centroid.

40. The method of embodiment 49, wherein the control dataset comprises coefficient of variation (CV) values of gene expression levels of the one or more genes in the control dataset, and the correlation to the centroid is weighted by the inverse of the CV values.

41. The method of any of embodiments 34-40, wherein the first differentiation state is earlier in a stem cell differentiation pathway than the second differentiation state.

42. The method of any of embodiments 34-41, wherein the second differentiation state is earlier in a stem cell differentiation pathway than the third differentiation state.

43. The method of any of embodiments 34-40, wherein the first differentiation state is in a cell differentiation pathway that is parallel to a cell differentiation pathway of the second differentiation state.

44. The method of any of embodiments 34-43, wherein the population of cells are selected from the group consisting of stem-cell derived cardiac muscle cells, stem-cell derived skeletal muscle cells, stem-cell derived kidney tubule cells, stem-cell derived red blood cell cells, stem-cell derived smooth muscle cells, stem-cell derived lung cells, stem-cell derived thyroid cells, stem-cell derived pancreatic cells, stem-cell derived epidermal cells, stem-cell derived pigment cells, and stem-cell derived neuronal cells.

45. The method of any of embodiments 34-44, wherein the population of cells are stem-cell derived neuronal cells.

46. The method of any of embodiments 34-45, wherein the second differentiation state is the differentiation state of a determined dopaminergic neuronal cell.

47. The method of any of embodiments 34-46, wherein the second differentiation state is the differentiation state of cells with fitness for engraftment.

48. The method of any of embodiments 34-47, wherein the first reference dataset comprises a representation of gene expression levels for one or more genes selected from Table E1.

49. The method of any of embodiments 34-48, wherein the second reference dataset comprises a representation of gene expression levels for one or more genes selected from Table E2.

50. The method of any of embodiments 34-49, wherein the first reference dataset comprises a representation of gene expression levels for at least 20 genes selected from Table E1.

51. The method of any of embodiments 34-50, wherein the second reference dataset comprises a representation of gene expression levels for at least 20 genes selected from Table E2.

52. The method of any of embodiments 34-51, wherein the first reference dataset comprises a representation of gene expression levels for at least 50 genes selected from Table E1.

53. The method of any of embodiments 34-52, wherein the second reference dataset comprises a representation of gene expression levels for at least 50 genes selected from Table E2.

54. The method of any of embodiments 34-53, wherein at least one of the first, second and third differentiation states is characterized using an in vitro assay.

55. The method of any of embodiments 34-54, wherein at least one of the first, second and third differentiation states is characterized using an in vivo assay.

56. The method of embodiment 55, wherein the in vivo assay comprises determining whether reference cells are capable of surviving, engrafting, and/or innervating tissue when administered to an animal or human subject.

57. The method of embodiment 55 or embodiment 56, wherein the in vivo assay comprises determining whether reference cells ameliorate or reverse symptoms of a neurodegenerative disease when implanted into an animal or human subject.

58. The method of embodiment 56 or embodiment 57, wherein the animal subject comprises an animal model of Parkinson's disease.

59. The method of any of embodiments 34-58, wherein the method further comprises calculating one or more additional similarity scores using one or more additional reference datasets, wherein:
each of the additional reference datasets comprises a representation of gene expression levels for one or more genes that are differentially expressed between cells at the second differentiation state and cells at an additional differentiation state;
the one or more additional similarity scores indicate whether the differentiation state of the test cells is more similar to the second differentiation state or to one of the one or more additional differentiation states, and
the classifying the differentiation state of the one or more test cells is based on the first similarity score, the second similarity score, and the one or more additional similarity scores.

60. The method of any of embodiments 34-59, wherein the representations of gene expression levels in the first reference dataset and/or the second reference dataset are obtained using machine learning.

61. The method of embodiment 60, wherein the machine learning comprises principal component analysis.

62. The method of any of embodiments 34-59, wherein the representations of gene expression levels in the first reference dataset and/or the second reference dataset comprise normalized gene expression levels.

63. The method of any of embodiments 34-62, wherein the method further comprises classifying the differentiation state of the one or more test cells as being the second differentiation state if the first and second similarity scores indicate that the differentiation state of the one or more test cells is more similar to the second differentiation state.

64. The method of any of embodiments 34-63, wherein the method further comprises selecting the in vitro population of cells comprising one or more test cells classified as having the second differentiation state as having the desired differentiation state.

65. A method for implanting a population of cells having a desired differentiation state into a subject, the method comprising:
(a) selecting a population of cells having a desired differentiation state using the method of any of embodiments 34-64; and
(b) implanting the population of cells into a subject.

66. The method of embodiment 65, wherein the cells having the desired differentiation state are determined dopaminergic cells, and the population of cells is implanted into a brain region of the subject.

67. The method of embodiment 65 or embodiment 66, wherein the cells having the desired differentiation state are from a culture of cells differentiated from pluripotent cells under conditions to neurally differentiate the cells.

68. A pharmaceutical composition comprising a pharmaceutical carrier and a population of cells having a desired differentiation state, wherein the cells are selected using the method of any of embodiments 34-64.

69. The pharmaceutical composition of embodiment 68, wherein the cells having the desired differentiation state are neuronal cells that are suitable for treatment of a neurodegenerative disease when implanted into a brain of a subject in need of such treatment.

70. The pharmaceutical composition of embodiment 68 or embodiment 69, wherein the neuronal cells comprise determined dopaminergic cells.

71. The pharmaceutical composition of any of embodiments 68-70, wherein the neuronal cells comprise engraftment-capable neuronal cells.

72. A method for training a machine learning model classifying the differentiation state of an in vitro population of cells, the method comprising:
(a) obtaining, for a plurality of reference populations of cells, gene expression levels for one or more genes that are differentially expressed between cells at a first differentiation state and cells at a second differentiation state and applying the gene expression levels as input to train a first machine learning model to predict if an in vitro population of cells comprises one or more test cells having a differentiation state that is more similar to the first differentiation state or to the second differentiation state; and
(b) obtaining, for a plurality of reference populations of cells, gene expression levels for one or more genes that are differentially expressed between cells at the second differentiation state and cells at a third differentiation state and applying the gene expression levels as input to train a second machine learning model to predict if an in vitro population of cells comprises one or more test cells having a differentiation state that is more similar to the second differentiation state or to the third differentiation state.

73. A method for training a machine learning model classifying the differentiation state of an in vitro population of cells, the method comprising:
(a) selecting one or more genes that are differentially expressed between cells at a first differentiation state and cells at a second differentiation state and applying expression levels of the selected genes for a plurality of reference populations of cells as input to train a first machine learning model to predict if an in vitro population of cells comprises one or more test cells having a differentiation state that is more similar to the first differentiation state or to the second differentiation state; and
(c) selecting one or more genes that are differentially expressed between cells at the second differentiation state and cells at a third differentiation state and applying expression levels of the selected genes for a plurality of reference populations of cells as input to train a second machine learning model to predict if an in vitro population of cells comprises one or more test cells having a differentiation state that is more similar to the second differentiation state or to the third differentiation state.

74. The method of embodiment 72 or embodiment 73, wherein the method further comprises obtaining gene expression levels for one or more genes that are expressed in cells at a control differentiation state, which control differentiation state may be the same as or different than one of the first, second or third differentiation states, and applying the gene expression levels as input to train a control machine learning model to predict if an in vitro population of cells comprises one or more test cells that are similar to the cells at the control differentiation state.

78. A method for selecting a population of cells predicted to exhibit neurite outgrowth following implantation in a brain region, comprising:
(a) obtaining a test dataset comprising gene expression levels of one or more genes selected from AC010247.2, ANKRD33B, APC2, AQP4, ASCL1, AURKB, BARHL2, CACNA1G, CAPN6, CBLN1, CCNB2, CDH1, CDH20, CHGA, COL1A1, COL1A2, COL22A1, COL4A1, CRABP1, DBX1, DCN, DCX, DDC, DOCK10, E2F4, EDNRB, ESRP1, EZH2, FABP7, FBLN1, FLRT3, FOXA2, FOXM1, GAP43, GFAP, GFRA1, GJA1, GLRA2, HES1, HES2, HES5, ITGA5, JPH4, LDHA, LIN28A, LIX1, LMX1A, LUM, NCAM1, NES, NEUROG2, NGFR, NKX2-2, NMNAT2, NPTX1, NR4A2, NR4A2 (NURR1), NSG2, NFYA, OLFM3, OLIG1, OLIG2, OTX2, P4HA1, PBX1, PDGFRA, PIEZO2, PITX3, PLP1, PMEL, PMP2, POSTN, POU2F2, PPP2R2B, PRTG, PTTG1, REST, RET, RFX4, RFX4, SALL4, SIN3A, SLC16A3, SLC18A2, SLC1A, SLC1A2, SLC1A3, SLC4A4, SMAD4, SNAP25, SOX10, SOX2, SOX9, STMN2, SUZ12, SV2B, SYN1, SYT1, SYT13, TH, TOP2A, TPH1, TPM2, and TXNIP for one or more test cells comprised in an in vitro population of cells; and
(b) applying the gene expression levels as input to a process configured to predict if the population of cells will exhibit neurite outgrowth following implantation in a brain region.

79. The method of any of embodiments 75-78, wherein the one or more genes comprise at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95 or more of AC010247.2, ANKRD33B, APC2, AQP4, ASCL1, AURKB, BARHL2, CACNA1G, CAPN6, CBLN1, CCNB2, CDH1, CDH20, CHGA, COL1A1, COL1A2, COL22A1, COL4A1, CRABP1, DBX1, DCN, DCX, DDC, DOCK10, E2F4, EDNRB, ESRP1, EZH2, FABP7, FBLN1, FLRT3, FOXA2, FOXM1, GAP43, GFAP, GFRA1, GJA1, GLRA2, HES1, HES2, HES5, ITGA5, JPH4, LDHA, LIN28A, LIX1, LMX1A, LUM, NCAM1, NES, NEUROG2, NGFR, NKX2-2, NMNAT2, NPTX1, NR4A2, NR4A2 (NURR1), NSG2, NFYA, OLFM3, OLIG1, OLIG2, OTX2, P4HA1, PBX1, PDGFRA, PIEZO2, PITX3, PLP1, PMEL, PMP2, POSTN, POU2F2, PPP2R2B, PRTG, PTTG1, REST, RET, RFX4, RFX4, SALL4, SIN3A, SLC16A3, SLC18A2, SLC1A, SLC1A2, SLC1A3, SLC4A4, SMAD4, SNAP25, SOX10, SOX2, SOX9, STMN2, SUZ12, SV2B, SYN1, SYT1, SYT13, TH, TOP2A, TPH1, TPM2, and TXNIP.

80. The method of any of embodiments 75-79, wherein the process comprises a machine learning model trained using gene expression levels of the one or more genes.

81. The method of embodiment 80, further comprising classifying the differentiation state of the one or more test cells based on one or more outputs of the machine learning model.

82. The method of embodiment 80, further comprising predicting if the test cells will exhibit neurite outgrowth following implantation in a brain region based on one or more outputs of the machine learning model.

83. A pharmaceutical composition comprising a pharmaceutical carrier and a population of neuronal cells, wherein the cells are selected using the method of any of embodiments 75-82.

84. An in vitro stem cell-derived neuronal cell population comprising cells that express one or more genes selected from the group consisting of CCNB2, AURKB, PTTG1, TOP2A, NEUROG2, HES1, REST, E2F4, FOXM1, SIN3A, NFYA, LIN28A, FLRT3, ITGA5, NES, SOX2, SOX9 and RFX4.

85. The in vitro stem-cell derived neuronal cell population of embodiment 84, wherein:
(1) at least one gene from the one or more genes is selected from the group consisting of CCNB2, AURKB, PTTG1, TOP2A, NEUROG2, HES1, REST, E2F4, FOXM1, SIN3A, NFYA, LIN28A, FLRT3, ITGA5; and
(2) at least one gene from the one or more genes is selected from the group consisting of NES, SOX2, SOX9 and RFX4.

86. The in vitro stem-cell derived neuronal cell population of embodiment 84 or embodiment 85, wherein at least one of the one or more genes is REST.

87. The in vitro stem-cell derived neuronal cell population of any of embodiments 84-86, wherein at least 50% of cells within the population express the one or more genes.

88. The in vitro stem-cell derived neuronal cell population of any of embodiments 84-86, wherein at least 60% of cells within the population express the one or more genes.

89. The in vitro stem-cell derived neuronal cell population of any of embodiments 84-86, wherein at least 70% of cells within the population express the one or more genes.

90. The in vitro stem-cell derived neuronal cell population of any of embodiments 84-86, wherein at least 80% of cells within the population express the one or more genes.

91. The in vitro stem-cell derived neuronal cell population of any of embodiments 84-86, wherein at least 90% of cells within the population express the one or more genes.

92. The in vitro stem-cell derived neuronal cell population of any of embodiments 84-91, wherein cells in the population express EN1 and CORIN.

93. The in vitro stem-cell derived neuronal cell population of any of embodiments 84-92, wherein less than 20% of the total cells in the composition express TH.

94. The in vitro stem-cell derived neuronal cell population of any of embodiments 84-93, wherein less than 10% of the total cells in the composition express TH.

95. The in vitro stem-cell derived neuronal cell population of any of embodiments 84-94, wherein the expression is RNA expression.

96. The in vitro stem-cell derived neuronal cell population of embodiment 95, wherein the RNA expression is measured by RNA sequencing.

97. The in vitro stem-cell derived neuronal cell population of any of embodiments 84-96 that has been differentiated in vitro from a pluripotent stem cell (PSC).

98. The in vitro stem-cell derived neuronal cell population of embodiment 97, wherein the one or more gene is a gene that is overexpressed in cells of the population compared to the iPSCs.

99. The in vitro stem-cell derived neuronal cell population of embodiment 97, wherein one or more gene is a gene that is overexpressed in cells of the population compared to cells of a precursor population differentiated from the iPSCs.

100. The in vitro stem-cell derived neuronal cell population of embodiment 97, wherein the one or more gene is a gene that is overexpressed in cells of the population compared to cells of a mature committed dopaminergic neuronal cell population differentiated from the iPSCs.

101. The in vitro stem-cell derived neuronal cell population of any of embodiments 98-100, wherein the overexpression is a positive log 2 fold change of greater than or greater than about 1.5-fold, 2.0-fold, 3.0-fold, 4.0-fold or 5-fold.

102. The in vitro stem-cell derived neuronal cell population of embodiment 97, wherein the one or more gene is a gene that is reduced in expression in cells of the population compared to the iPSCs.

103. The in vitro stem-cell derived neuronal cell population of embodiment 97, wherein one or more gene is a gene that is reduced in expression in cells of the population compared to cells of a precursor population differentiated from the iPSCs.

104. The in vitro stem-cell derived neuronal cell population of embodiment 97, wherein the one or more gene is a gene that is reduced in expression in cells of the population compared to cells of a mature committed dopaminergic neuronal cell population differentiated from the iPSCs.

105. The in vitro stem-cell derived neuronal cell population of embodiment 100 or embodiment 104, wherein the mature committed dopaminergic neuronal cells express LMX1A and/or NR4A2 (NURR1).

106. The in vitro stem-cell derived neuronal cell population of embodiment 105, wherein among cells in the committed dopaminergic neuronal cell population, at least 40%, at least 50%, at least 60%, at least 70%, or at least 80% of the cells express LMX1A and/or NR4A2.

107. The in vitro stem-cell derived neuronal cell population of any of embodiments 102-106, wherein the reduced expression is a negative log 2 fold change of greater than or greater than about 1.5-fold, 2.0-fold, 3.0-fold, 4.0-fold or 5-fold.

108. The in vitro stem-cell derived neuronal cell population of any of embodiments 84-107, wherein less than 30%, less than 20%, or less than 10% of the cells in the population express LMX1A and/or NR4A2.

109. The in vitro stem-cell derived neuronal cell population of any of embodiments 84-108, wherein cells in the population are capable of engrafting in and innervating other cells in vivo.

110. The in vitro stem-cell derived neuronal cell population of any of embodiments 84-109, wherein cells in the population are capable of exhibiting neurite outgrowth when administered to the brain of a subject.

111. The in vitro stem-cell derived neuronal cell population of any of embodiments 84-110, wherein cells in the population are capable of producing dopamine and optionally do not produce or do not substantially produce norepinephrine.

112. The in vitro stem-cell derived neuronal cell population of any of embodiments 84-111, wherein the population comprises at least 5 million total cells, at least 10 million total cells, at least 15 million total cells, at least 20 million total cells, at least 30 million total cells, at least 40 million total cells, at least 50 million total cells, at least 100 million total cells, at least 150 million total cells, or at least 200 million total cells.

113. The in vitro stem-cell derived neuronal cell population of any of embodiments 84-112, wherein the population comprises between at or about 5 million total cells and at or about 200 million total cells, between at or about 5 million total cells and at or about 150 million total cells, between at or about 5 million total cells and at or about 100 million total cells, between at or about 5 million total cells and at or about 50 million total cells, between at or about 5 million total cells and at or about 25 million total cells, between at or about 5 million total cells and at or about 10 million total cells, between at or about 10 million total cells and at or about 200 million total cells, between at or about 10 million total cells and at or about 150 million total cells, between at or about 10 million total cells and at or about 100 million total cells, between at or about 10 million total cells and at or about 50 million total cells, between at or about 10 million total cells and at or about 25 million total cells, between at or about 25 million total cells and at or about 200 million total cells, between at or about 25 million total cells and at or about 150 million total cells, between at or about 25 million total cells and at or about 100 million total cells, between at or about 25 million total cells and at or about 50 million total cells, between at or about 50 million total cells and at or about 200 million total cells, between at or about 50 million total cells and at or about 150 million total cells, between at or about 50 million total cells and at or about 100 million total cells, between at or about 100 million total cells and at or about 200 million total cells, between at or about 100 million total cells and at or about 150 million total cells, or between at or about 150 million total cells and at or about 200 million total cells.

114. The in vitro stem-cell derived neuronal cell population of any of embodiments 84-113, wherein at least about 70%, 75%, 80%, 85%, 90%, or 95% of the total cells in the composition are viable.

115. A pharmaceutical composition comprising a pharmaceutical carrier and the in vitro stem-cell derived neuronal cell population of any of embodiments 84-114.

116. The pharmaceutical composition of embodiment 68, embodiment 83 and embodiment 115 wherein the composition comprises a cryoprotectant.

117. The pharmaceutical composition of embodiment 116, wherein the cryoprotectant is selected from among the group consisting of glycerol, propylene glycol, and dimethyl sulfoxide (DMSO).

118. The pharmaceutical composition of any one of embodiments 68, 83 and 115-117, wherein the composition is for use in treatment of a neurodegenerative disease or condition in a subject, optionally wherein the neurodegenerative disease or condition comprises a loss of dopaminergic neurons.

119. The pharmaceutical composition of any one of embodiments 68, 83 and 115-118, wherein the neurodegenerative disease or condition comprises a loss of dopaminergic neurons in the substantia nigra, optionally in the SNc.

120. The pharmaceutical composition of embodiment 118 or embodiment 119, wherein the neurodegenerative disease or condition is Parkinson's disease.

121. The pharmaceucal composition of any of embodiments 118-120, wherein the neurodegenerative disease or condition is a Parkinsonism.

122. A method of treatment, comprising implanting in a brain region of a subject in need thereof a therapeutically effective amount of the pharmaceutical composition of any one of embodiments 68, 83 and 115-121.

123. The method of embodiment 122, wherein the number of cells implanted in the subject is between about 0.25×10⁶ cells and about 20×10⁶ cells, between about 0.25×10⁶ cells and about 15×10⁶ cells, between about 0.25×10⁶ cells and about 10×10⁶ cells, between about 0.25×10⁶ cells and about 5×10⁶ cells, between about 0.25×10⁶ cells and about 1×10⁶ cells, between about 0.25×10⁶ cells and about 0.75×10⁶ cells, between about 0.25×10⁶ cells and about 0.5×10⁶ cells, between about 0.5×10⁶ cells and about 20×10⁶ cells, between about 0.5×10⁶ cells and about 15×10⁶ cells, between about 0.5×10⁶ cells and about 10×10⁶ cells, between about 0.5×10⁶ cells and about 5×10⁶ cells, between about 0.5×10⁶ cells and about 1×10⁶ cells, between about 0.5×10⁶ cells and about 0.75×10⁶ cells, between about 0.75×10⁶ cells and about 20×10⁶ cells, between about 0.75×10⁶ cells and about 15×10⁶ cells, between about 0.75×10⁶ cells and about 10×10⁶ cells, between about 0.75×10⁶ cells and about 5×10⁶ cells, between about 0.75×10⁶ cells and about 1×10⁶ cells, between about 1×10⁶ cells and about 20×10⁶ cells, between about 1×10⁶ cells and about 15×10⁶ cells, between about 1×10⁶ cells and about 10×10⁶ cells, between about 1×10⁶ cells and about 5×10⁶ cells, between about 5×10⁶ cells and about 20×10⁶ cells, between about 5×10⁶ cells and about 15×10⁶ cells, between about 5×10⁶ cells and about 10×10⁶ cells, between about 10×10⁶ cells and about 20×10⁶ cells, between about 10×10⁶ cells and about 15×10⁶ cells, or between about 15×10⁶ cells and about 20×10⁶ cells.

124. The method of embodiment 122 or embodiment 123, wherein the subject has a neurodegenerative disease or condition.

125. The method of any one of embodiments 122-124, wherein the neurodegenerative disease or condition comprises the loss of dopaminergic neurons.

126. The method of any one of embodiments 122-125, wherein the subject has lost at least 50%, at least 60%, at least 70%, or at least 80% of dopaminergic neurons.

127. The method of any one of embodiments 122-126, wherein the subject has lost at least 50%, at least 60%, at least 70%, or at least 80% of dopaminergic neurons in the substantia nigra (SN), optionally in the SN pars compacta (SNc).

128. The method of any one of 122-127, wherein the neurodegenerative disease or condition is a Parkinsonism.

129. The method of any one of embodiments 122-128, wherein the neurodegenerative disease or condition is Parkinson's disease.

130. The method of any of embodiments 122-129, wherein the brain region is the substantia nigra.

131. The method of any one of embodiments 122-130, wherein the implanting is by stereotactic injection.

132. The method of any one of embodiments 122-131, wherein the cells of the pharmaceutical composition are autologous to the subject.

### VIII. EXAMPLES

The following examples are included for illustrative purposes only and are not intended to limit the scope of the invention.

### Example 1: Machine Learning Method for Identifying Cells at an Intermediate Differentiation State

A machine learning method for identifying cell populations having a desired differentiation state was developed. To do so, gene expression levels of reference cell populations at various differentiation states were used as training data, and multiple machine learning models each trained to discriminate between cell populations of two different differentiation states were developed.

Training data included RNA sequencing (RNAseq) data collected from cell populations at earlier, intermediate, and later differentiation states (n=8 per state) during their culture in an exemplary differentiation protocol involving the in vitro culture of induced pluripotent stem cells (iPSCs) under conditions to neurally differentiate the cells to dopaminergic neurons. Model training procedures using this data are described in further detail below. Briefly, expression levels across reference cell populations were used to develop a cutoff value for a novelty score indicating whether expression levels of a test cell population are dissimilar to those of the reference cell populations. In addition, a first machine learning model was trained to discriminate between test cell populations having expression levels similar to the earlier-state reference cell populations (e.g., cells at day 13 of the differentiation protocol) or the intermediate-state reference cell populations (e.g., cells at day 18 of the differentiation protocol). A separate, second machine learning model was trained to discriminate between test cell populations having expression levels similar to the later-state reference cell populations (e.g., cells at day 25 of the differentiation protocol) or the intermediate-state reference cell populations (e.g., cells at day 18 of the differentiation protocol).

Following model training, validation analyses were performed using RNAseq data from multiple sets of test cell populations. These results are also described below. Overall, the described methods resulted in the identification of determined dopaminergic neuronal cells with 89.2% sensitivity (n=74) and 95.9% specificity (n=98) in test cell populations not used in model training.

### A. In Vitro Cell Culture

For cell culture, dermal fibroblasts obtained from punch biopsies were isolated and reprogrammed. iPSCs were differentiated on Geltrex using a modified version of a previously published dual-SMAD inhibition protocol (Kriks et al., Nature 2011; 480:547-551). iPSCs were dissociated and seeded in maintenance medium supplemented with a rho kinase inhibitor before switching to differentiation medium 24 hours later. The following were added to the differentiation medium to induce floor plate precursor differentiation: LDN193189 (days 1-13), SB431542 (days 1-5), CHIR99021 (days 3-13), Purmorphamine (days 2-7), and sonic hedgehog C25II (days 2-7). For earlier-state reference cell populations, cultures were dissociated on day 13 of differentiation, and cell suspensions were cryopreserved.

After day 13 of differentiation, basal medium was switched to medium supplemented with BDNF, GDNF, ascorbic acid, dBcAMP, TGFB3, and DAPT. On day 16 of differentiation, cells were passaged and reseeded on poly-1-ornithine-, laminin-, and fibronectin-coated dishes in medium containing rho kinase inhibitor. For intermediate-state reference cell populations, cultures were dissociated and cryopreserved on day 18 of differentiation. For later-state reference cell populations, parallel cultures were passaged at day 20; reseeded on poly-1-ornithine-, laminin-, and fibronectin-coated dishes; and cultured to day 25, when they were dissociated and cryopreserved.

### B. Behavioral Assay

The reference cell populations (earlier-, intermediate-, and later-state reference cell populations) were tested for their effects on Parkinson's disease (PD) symptoms following transplantation. To do so, a PD rat model was used. In this model, rats received unilateral stereotaxic injection of 6-hydroxydopamine (6-OHDA) into the substantia nigra or the medial forebrain bundle. This lesioning led to asymmetric dopamine discharge after amphetamine treatment that caused lesioned rats to circle in one direction when moving. After baseline circling behavior was measured in lesioned rats, reference cell populations were transplanted into the lesioned hemisphere. Rats were then periodically tested for amphetamine-induced circling.

Six to eight weeks after transplant of intermediate-state reference cell populations, but not earlier- or later-state reference cell populations, the net number of amphetamine-induced rotations was reduced to zero. This result showed that transplantation of developmentally determined dopaminergic cells (e.g., cells at day 18 of the differentiation protocol) led to the reversal or amelioration of PD symptoms.

### C. RNA Sequencing Pre-Processing

Total RNA libraries for paired-end sequencing were prepared from all reference cell populations (earlier-, intermediate-, and later-state reference cell populations). To do so, total RNA was extracted from approximately 1 million cells in culture using a mirVANA™ miRNA isolation kit (Invitrogen) following the manufacturer's protocol. One hundred and fifty base pair (150 bp) paired-end sequencing was performed on the Illumina HiSeq 2000 platform (Illumina, San Diego, CA).

The nf-core-rnaseq v1.4.2 pipeline (Ewels et al., Nature Biotechnology 2020; 38(3):276-278) was used for sample preprocessing. Fastq files were interpreted and processed using the Salmon pseudo-aligner (salmon version 1.1.0; Patro et al., Nature Methods 2017; 14(4):417-419) using default parameters with no additional flags and the GENCODE human reference genome (release 32) as the genome index. Transcripts were aggregated (summed) for each gene so that the sum of all transcripts per gene was the gene-level count.

### D. Novelty Score Training

RNAseq read count data from all reference cell populations (earlier-, intermediate-, and later-state reference cell populations) were normalized to counts-per-million (CPM) and log₂-transformed. From this data, genes having median expression level greater than 10 CPM were selected. The mean, standard deviation, and coefficient of variation (CV) of the expression levels of the selected genes (approximately 11,500 genes) were calculated across reference cell populations.

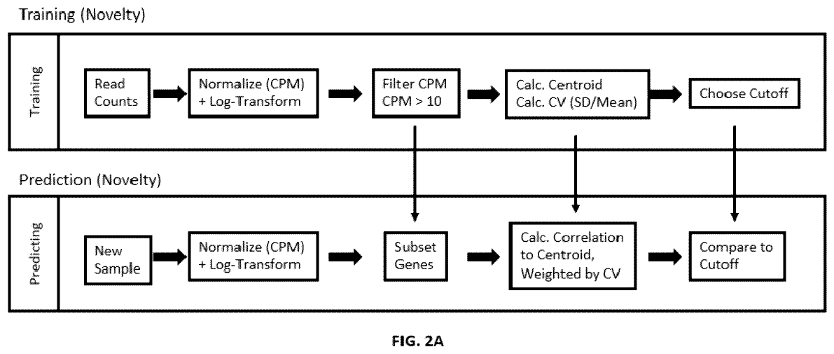

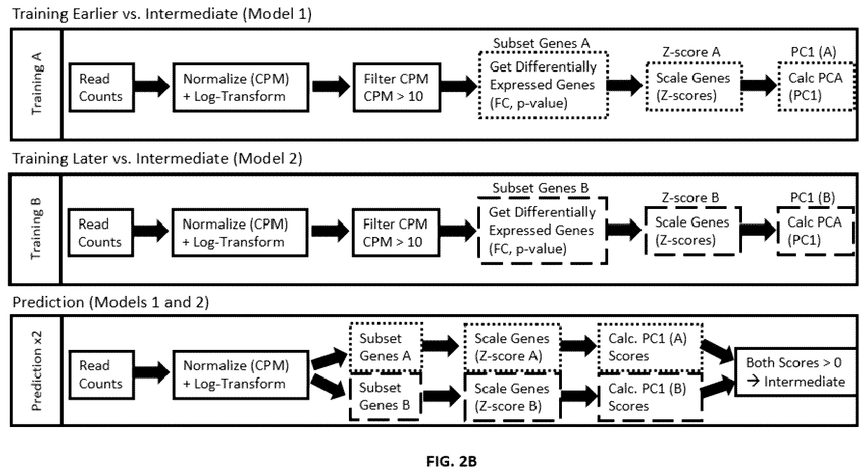

Next, a cutoff value for a novelty score indicating if a test cell population has expression levels dissimilar to those of the reference cell populations was established. To do so, for each reference cell population, a weighted correlation of expression levels across the selected genes (those having median CPM greater than 10) to the mean expression levels across reference cell populations was calculated. For correlation calculation, genes were weighted by 1/CV values. Based on these correlation values, a novelty score cutoff was set such that test cell populations with (one minus weighted correlation values) greater than 0.15 would be identified as dissimilar to the reference cell populations. These procedures for developing the novelty score, as well as its application to test cell populations, are shown in FIG. 2A.

### E. Model 1 Training: Earlier-State Vs. Intermediate-State Cell Populations

Expression levels of earlier-state (e.g., day-13) and intermediate-state (e.g., day-18) reference cell populations were used to train a principal component analysis (PCA) model. RNAseq read count data from these reference cell populations were normalized to CPM and log₂-transformed. From this data, genes having median expression levels greater than 10 CPM were selected. The selected genes were then further filtered for genes that were differentially expressed between earlier-state (e.g., day-13) and intermediate-state (e.g., day-18) reference cell populations. Statistical analysis of differential gene expression was performed using empirical Bayes estimation (R edgeR package). These differentially expressed genes had a minimum absolute log₂ fold-change (FC) of 3 with an associated adjusted p-value of less than 0.001. Out of approximately 11,500 genes, 347 genes satisfying these criteria were identified. Expression levels of the differentially expressed genes were normalized to Z-scores and applied as input for PCA. Weights for calculating Principal Component 1 (PC1) values were extracted for later use. PC1 explained 83.56% of data variance. Based on PC1 values of the reference cell populations, a PC1 cutoff was set such that test cell populations with PC1 values greater than 0 would be identified as having expression levels similar to intermediate-state (e.g., day-18) reference cell populations. These procedures for training the model and applying it to test cell populations are shown in FIG. 2B.

### F. Model 2 Training: Later-State Vs. Intermediate-State Cell Populations

A separate, second PCA model was trained as described above, but instead using expression levels of later-state (e.g., day-25) and intermediate-state (e.g., day-18) reference cell populations. PC1 explained 79.79% of data variance. As above, genes selected for differential expression between later-state (e.g., day-25) and intermediate-state (e.g., day-18) cell populations had a minimum median CPM of 10 and a minimum absolute log₂ FC of 3 with an associated adjusted p-value of less than 0.001. Out of approximately 11,500 genes, 365 genes satisfying these criteria were identified. A PC1 cutoff for the second model was set such that test cell populations with PC1 values greater than 0 would be identified as similar to intermediate-state (e.g., day-18) reference cell populations. These procedures for training the model and applying it to test cell populations are shown in FIG. 2B.

### G. Validation

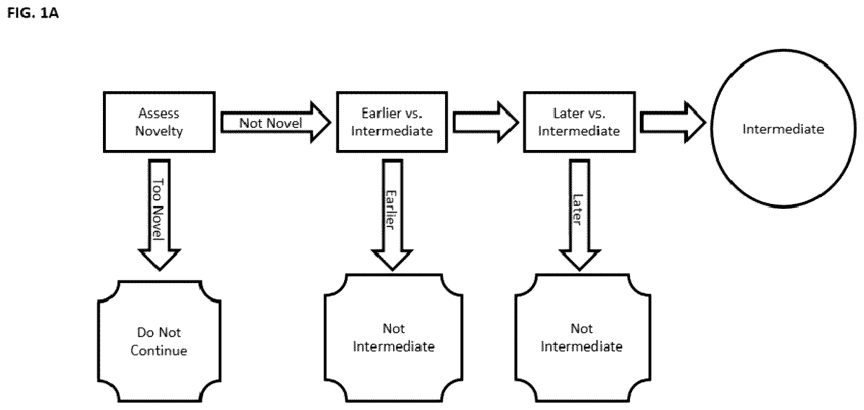

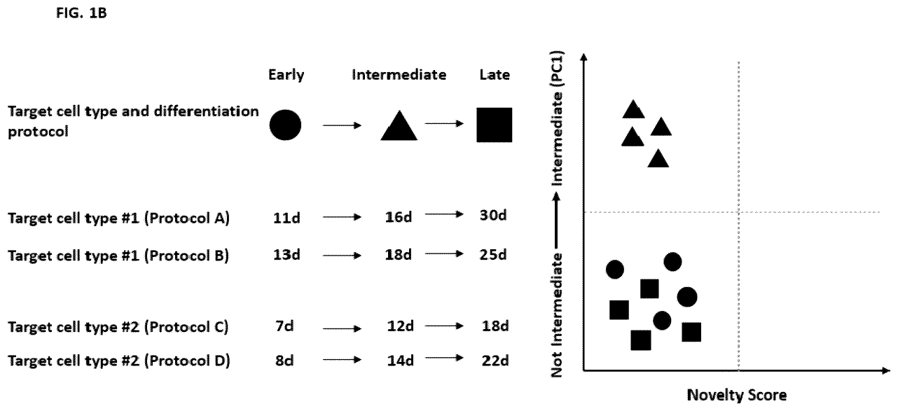

The novelty score and two PC1 cutoffs were validated using RNAseq data from test cell populations not used for model training. A decision tree for the testing procedure is shown in FIG. 1A. First, novelty scores for each test cell population were determined using the same set of genes selected during training (median CPM greater than 10). For each test cell population, a weighted correlation of expression levels across selected genes to the mean expression levels across reference cell populations was calculated. For correlation calculation, genes were weighted based on the 1/CV values of the reference cell populations. Test cell populations with novelty scores (one minus weighted correlation) greater than 0.15 were not analyzed further.

Test cell populations with sufficiently low novelty scores were subjected to further analysis. Per test cell population, PC1 values for each PCA model were determined using the same differentially expressed genes selected during training. Prior to PC1 value calculation, expression levels of the test cell populations were normalized to z-scores using the mean and standard deviation of the reference cell populations, after which the PC1 weights calculated during training were used to calculate PC1 values for each test cell population. Test cell populations with both PC1 values greater than 0 were identified as similar to intermediate-state (e.g., day-18) reference cell populations.

The novelty score and two PC1 cutoffs were validated using different sets of test cell populations, including (i) cell populations harvested at different time points during in vitro culture under conditions to neurally differentiate the cells to dopaminergic neurons, (ii) test cell populations that were generated using an alternative differentiation protocol that was not used to produce the reference cell populations, and (iii) test cell populations of glial cells.

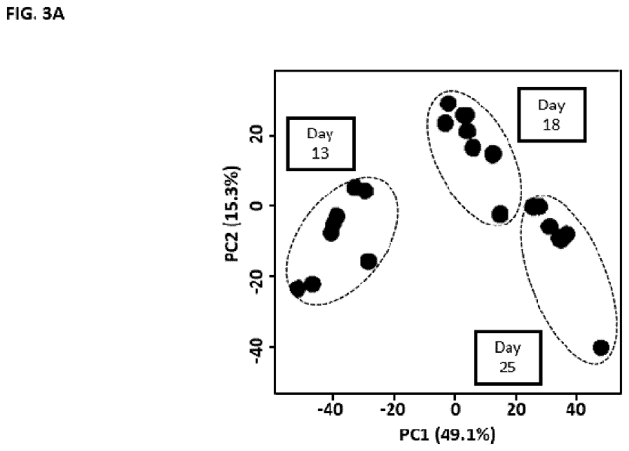

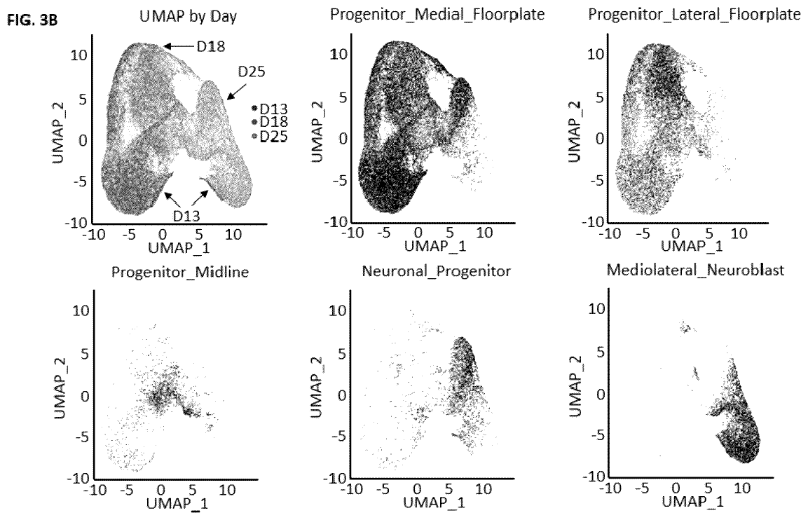

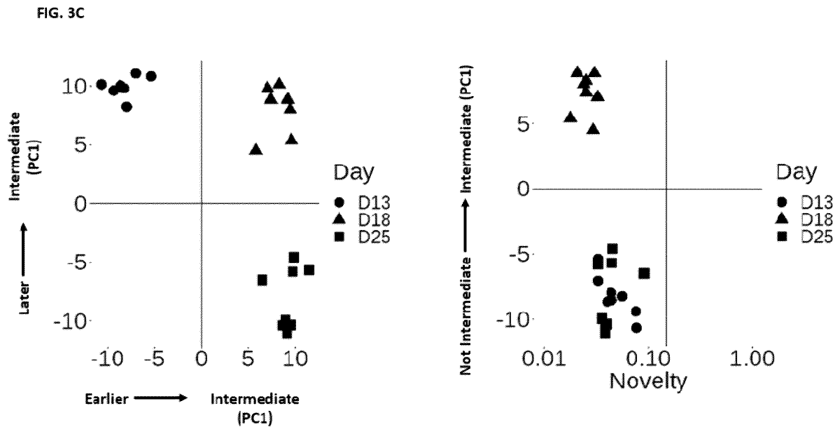

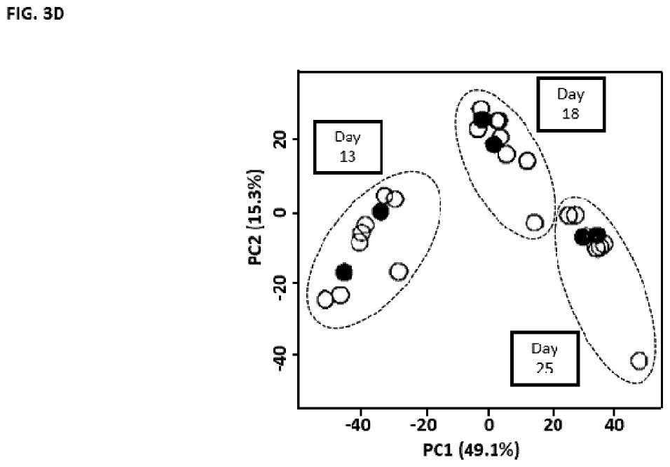

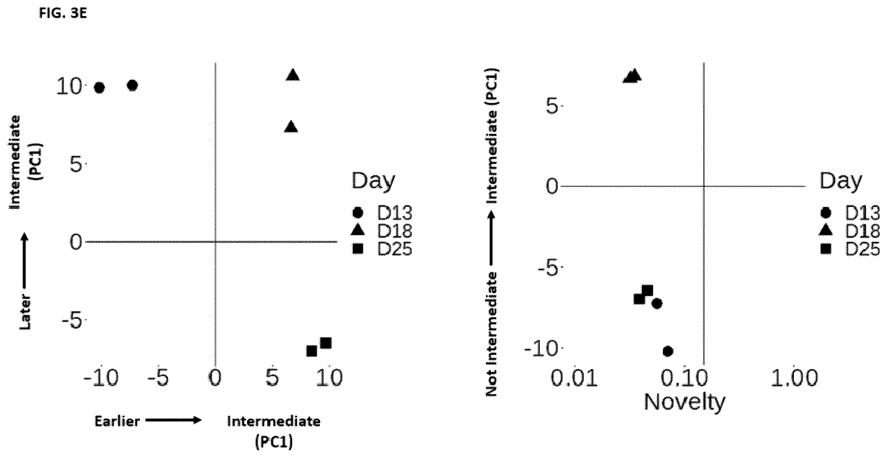

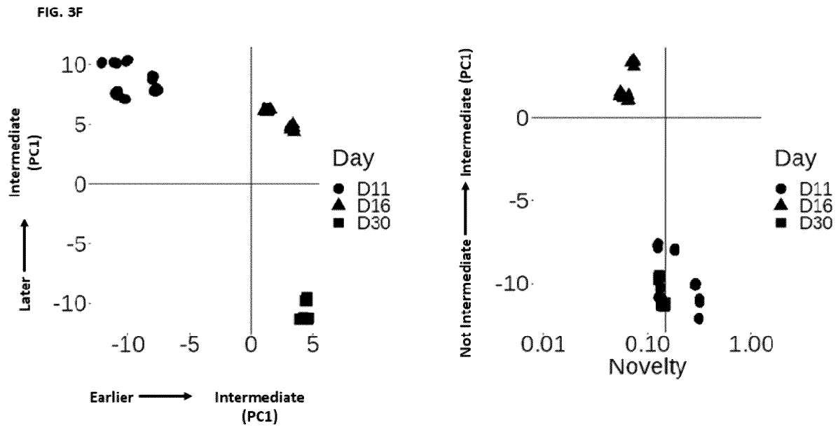

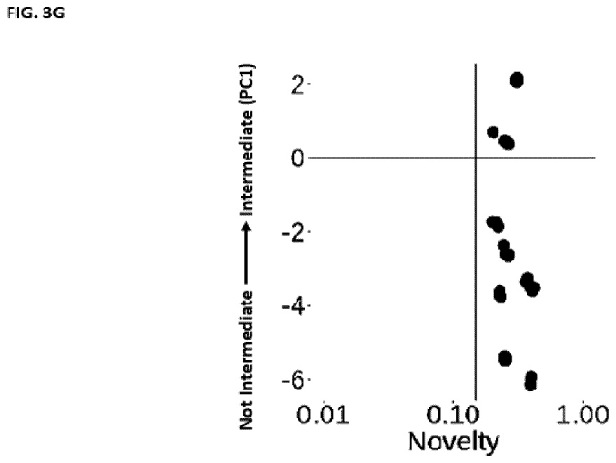

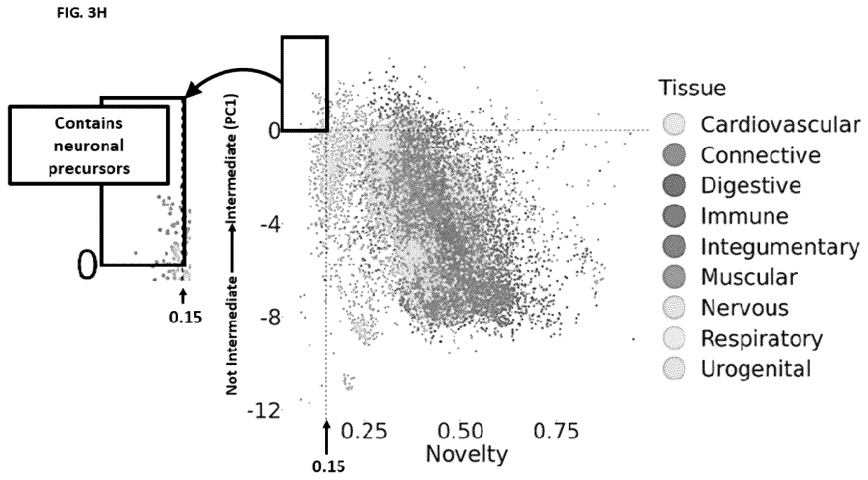

Results for reference cell populations used for training are shown in FIG. 3A-3C. Validation results for three different sets of test cell populations are shown in FIG. 3D-3H. Results shown in FIG. 3A-3F include those for cell populations harvested at different time points during in vitro culture under conditions to neurally differentiate the cells to dopaminergic neurons.

FIG. 3A shows the results of a single PCA model trained using gene expression levels from all of the reference cell populations (e.g., all of day-13, day-18, and day-25 reference cell populations). As shown in FIG. 3A, reference cell populations that were collected at different states segregated from one another based on PC1 and PC2 values, which explained 49.1% and 15.3% of the variance, respectively, for the single PCA model.

FIG. 3B shows the results of uniform manifold approximation and projection (UMAP) nonlinear dimensionality reduction on single-cell RNA sequencing gene expression levels for cells from some of the reference cell populations. Inferred cell types for each cell were determined using another reference transcriptomic dataset that included single-cell transcriptomic data from embryonic human midbrain samples and predicted cell type labels for its individual cells (see La Manno et al. (2016), Cell 167(2): 566-580). As shown in FIG. 3B, cells from reference cell populations in earlier (e.g., day 13) or intermediate states (e.g., day 18) of differentiation were predicted to be medial or lateral floorplate progenitor cells. Cells from reference cell populations in an intermediate state (e.g., day 18) of differentiation were predicted to be midline progenitor cells. Cells from reference cell populations in an intermediate state (e.g., day 18) of differentiation also had transcriptomes enriched for ontological hallmarks of dopaminergic neuronal precursor cells, including dopamine secretion, amine metabolism, regulation of membrane potential, and regulation of neuron projection development. Cells from reference cell populations in intermediate (e.g., day 18) or later states (e.g., day 25) of differentiation were predicted to be neuronal progenitor cells. Cells from reference cell populations in a later state (e.g., day 25) of differentiation were predicted to be mediolateral neuroblast cells.

FIG. 3C shows the results of the two separately-trained PCA models. As shown in FIG. 3C (left panel), reference cell populations that were collected at an intermediate state (e.g., day 18) of differentiation in culture (triangles) had both PC1 values greater than 0 and had PC1 values distinguishable from reference cell populations collected at an earlier state (e.g., day 13; circles) or a later state (e.g., day 25; squares) of differentiation in culture. All reference cell populations had novelty scores below 0.15 (right panel). Y-axis values in the right panel of FIG. 3C reflect the minimum PC1 value between models.

Similar results are shown in FIG. 3D-3E for a set of test cell populations. For these test cell populations, RNAseq data was also collected at earlier (e.g., day 13), intermediate (e.g., day 18), and later (e.g., day 25) states of differentiation during in vitro culture. FIG. 3D shows PC1 and PC2 values for the test cell populations based on the single PCA model also shown in FIG. 3A, with the test cell populations shown in shaded circles and the reference cell populations shown in unshaded circles. FIG. 3E shows, for the test cell populations, similar results to those shown in FIG. 3C. These results validate that the trained models were able to accurately identify intermediate-state (e.g., day 18) cell populations not included during model training.

FIG. 3F shows results for a set of test cell populations that were generated using an alternative differentiation protocol that was not used to produce either the reference cell populations from training or the test cell populations with results shown in FIG. 3D-3E. This alternative differentiation protocol is described, for example, in Kim et al., Cell Stem Cell (2021) 28(2):P343-355.E5. Data from test cell populations in this alternative differentiation protocol were also collected at earlier, intermediate, and later differentiation states (e.g., day 11, day 16, and day 30 of culture, respectively). As shown in FIG. 3F (left panel), test cell populations that were collected at the intermediate state (e.g., day 16) of the alternative differentiation protocol (triangles) also had both PC1 values greater than 0 and had PC1 values distinguishable from test cell populations collected at the earlier state (e.g., day 11; circles) or later state (day 30; squares) of the alternative differentiation protocol. These intermediate-state test cell populations also had novelty scores less than 0.15 (right panel). These results indicate that the trained models were able to generalize to and accurately identify intermediate-state cell populations produced using alternative differentiation protocols.

FIG. 3G shows results for test cell populations of glial cells. As shown, all glial test cell populations had a novelty score greater than 0.15. These results indicate that the novelty score is effective in identifying cell populations having an alternative differentiation fate (e.g., glial, rather than neuronal).

FIG. 3H shows results for test cell populations of various cell types. Bulk RNA sequencing gene expression levels for the test cell populations were obtained from the ARCHS4 data set described in Lachmann et al. (2018), Nature Communications 9: 1366. Only nervous system cells had a novelty score less than 0.15, and of all 30,000 test cell populations, only 42 test cell populations had gene expression levels with novelty score less than 0.15 and minimum PC1 values greater than 0. ARCHS4 annotation indicated that all of these 42 test cell populations were neuronal, with many annotated as being dopaminergic neuronal precursor cell populations.

### H. Conclusion

Overall, the developed machine learning method was able to accurately identify cell populations based on gene expression levels. The novelty score and corresponding cutoff value was effective in screening test cell populations dissimilar to reference cell populations used in training. In addition, the two models leveraged by the method together identified cells harvested at an intermediate state (e.g., day 18, versus day 13 or day 25) with high specificity and sensitivity, including for test cell populations produced using alternative differentiation protocols. These results indicate the ability of the developed method to successfully identify cell populations having a desired differentiation state, for instance an intermediate (e.g., determined) differentiation state, versus an earlier (e.g., precursor) or later (e.g., committed) differentiation state.

### Example 2: Identifying Intermediate-State Cells Using Differentially Expressed Genes

Gene expression levels were analyzed to identify genes that were significantly differentially expressed between earlier-state (e.g., day-13, n=2) and intermediate-state (e.g., day-18, n=2) cell populations and between later-state (e.g., day-25, n=2) and intermediate-state cell populations. Gene expression levels were collected during the culture of reference cell populations in an exemplary differentiation protocol involving the in vitro culture of induced pluripotent stem cells (iPSCs) under conditions to neurally differentiate the cells to dopaminergic neurons.

### A. In Vitro Cell Culture

For cell culture, dermal fibroblasts obtained from punch biopsies were isolated and reprogrammed. Dermal punch biopsies (3 mm) were obtained from two individuals diagnosed with idiopathic Parkinson's Disease (PD). Dermal fibroblasts were isolated as described in Glenn et al. (In: Loring and Peterson eds. Hum. Stem Cell Man., London: Elsevier Inc., 2012:129-141). Isolated dermal fibroblasts were reprogrammed using the Sendai CytoTunel-iPS Reprogramming Kit (ThermoFisher). Multiple iPSC clones from each cell line were isolated, expanded, and banked as previously described in Boland et al. (Brain 2017; 140:582-598).

iPSCs were differentiated on Geltrex (Life Technologies, 1:200 dilution) using a modified version of a previously published dual-SMAD inhibition protocol (Kriks et al., Nature 2011; 480:547-551). iPSCs were dissociated with Accutase® (Gibco) and seeded as single cells at a concentration of 200k cells/cm² in maintenance medium (Essential 8 medium, ThermoFisher) supplemented with a rho kinase inhibitor (Stemgent, 04-0012-02, 1 μM) before switching to differentiation medium 24 hours later. Differentiation medium consisted of a 1:1 mix of DMEM/F-12 and Neurobasal medium containing 1×N2/B27, GlutaMax™, and MEM-NEAA (all from ThermoFisher). Differentiation medium contained varying amounts of KnockOut™ Serum Replacement (ThermoFisher) starting at 5% on the first 2 days of differentiation, decreasing to 2% through day 10 of differentiation. The following were added to the differentiation medium to induce floor plate precursor differentiation: LDN193189 (days 1-13; 100 nM, Stemgent), SB431542 (days 1-5, 2 μM, Tocris), CHIR99021 (days 3-13, 2 μM, Stemgent), Purmorphamine (days 2-7, 2 μM, Calbiochem), and sonic hedgehog C25II (days 2-7, 100 ng/mL, R&D Systems). For earlier-state reference cell populations, cultures were treated on day 13 of differentiation with Accutase®, and single-cell suspensions were cryopreserved in CryoStor CS10 cryopreservation medium (Stemcell Technologies) according to the manufacturer's instructions.

After day 13 of differentiation, basal medium was switched to Neurobasal medium containing 1×N2/B27, GlutaMax™, and MEM-NEAA supplemented with BDNF (20 ng/mL, R&D Systems), GDNF (20 ng/mL, Peprotech), ascorbic acid (0.2 mM, Sigma-Aldrich), dBcAMP (0.5 mM, Sigma-Aldrich), TGFB3 (1 ng/mL, R&D Systems), and DAPT (10 PM, Tocris). On day 16 of differentiation, cells were passaged using Dispase/collagenase (Roche) and DNase (Worthington Biomedical) and reseeded at a 1:2 passage ratio on poly-1-ornithine-(Sigma), laminin-(Roche), and fibronectin-(Sigma) coated dishes in medium containing rho kinase inhibitor.

For intermediate-state reference cell populations, cultures were treated on day 18 of differentiation with Accutase®, and single-cell suspensions were cryopreserved in CryoStor CS10 cryopreservation medium (Stemcell Technologies) according to the manufacturer's instructions. For later-state reference cell populations, parallel cultures were passaged at day 20 using Accutase®; reseeded at a 1:1 ratio in poly-1-ornithine (Sigma), laminin (Roche), and fibronectin (Sigma) coated dishes; and cultured to day 25, when they were dissociated with Accutase®, and single-cell suspensions were cryopreserved. Parallel cultures of each line were allowed to mature further for 12 weeks in maturation medium without DAPT. Laminin was supplemented into the medium once a week at a concentration of 1 μg/ml to maintain attachment to the surface.

### B. Behavioral Assay

Intermediate- and later-state reference cell populations were tested for their effects on Parkinson's disease (PD) symptoms following transplantation. To do so, a PD rat model was used. In this model, rats received unilateral stereotaxic injection of 6-hydroxydopamine (6-OHDA) into the substantia nigra or the medial forebrain bundle. This lesioning led to asymmetric dopamine discharge after amphetamine treatment that caused lesioned rats to circle in one direction when moving. In this study, after baseline circling behavior was measured in lesioned rats, reference cell populations were transplanted into the lesioned hemisphere. Rats were then periodically tested for amphetamine-induced circling.

Rotational bias was restored twenty-four weeks after transplant of intermediate-state reference cell populations, but not after transplant of later-state reference cell populations. This result showed that transplantation of developmentally determined dopaminergic cells (e.g., cells at day 18 of the differentiation protocol) led to the reversal or amelioration of PD symptoms.

### C. Immunohistological Analysis

Immunohistological analysis was used to quantify the number of engrafted human cells (HuNu+), and cells positive for mature DA neurons (TH+, AADC+, GIRK2+) in grafts. In summary, behavioral recovery did not correlate with the number of HuNu+ cells, graft volume, total number of TH+ cells, AADC+ cells, GIRK2+ cells, or density of TH+ cells.

However, manual counting of fibers revealed 4-fold higher average density of projections from day 18 grafts compared to day 25 grafts. Improvements in amphetamine-induced rotational bias correlated significantly with greater overall neurite outgrowth (r=−0.754, p<0.001), greater fiber projections into the lateral neostriatum (r=−0.696, p≤0.001), and greater fiber outgrowth into the medial striatum (r=−0.723, p<0.001).

### D. RNA Sequencing Pre-Processing

Total RNA libraries for paired-end sequencing were prepared from the reference cell populations. To do so, total RNA was extracted from approximately 1 million cells in culture using a mirVANA™ miRNA isolation kit (Invitrogen) following the manufacturer's protocol. All reference cell populations achieved a minimum RNA Integrity Number (RIN) of 9.0 prior to sequencing. One hundred and fifty base pair (150 bp) paired-end sequencing was performed on the Illumina HiSeq 2000 platform (Illumina, San Diego, CA).

The nf-core-rnaseq v1.4.2 pipeline (Ewels et al., Nature Biotechnology 2020; 38(3):276-278) was used for sample preprocessing. Fastq files were interpreted and processed using the Salmon pseudo-aligner (salmon version 1.1.0; Patro et al., Nature Methods 2017; 14(4):417-419) using default parameters with no additional flags and the GENCODE human reference genome (release 32) as the genome index. Transcripts were aggregated (summed) for each gene so that the sum of all transcripts per gene was the gene-level count. Count data were used for differential expression analyses.

### E. Gene Selection

For differential expression (DE) analysis, genes were pre-filtered by removing the bottom 40% quantile of genes sorted by their row sums (Anders S et al., Genome Biology 2010; 11(10):R106). This resulted in 32,836 genes remaining in the downstream DE analyses. Dispersion estimates were calculated sequentially by estimateGLMCommonDisp, estimateGLMTrendedDisp, and estimateGLMTagwiseDisp methods (Robinson et al., Bioinformatics 2009; 26(1):139-140). The differential expression contrast was constructed using the makeContrasts command, and the gene-wise negative binomial generalized linear model was fit using glmFit. Likelihood ratio tests were performed using the glmLRT test while applying the Benjamini and Hochberg multiple test correction (Benjamini et al., Journal of the Royal Statistical Society Series B (Methodological) 1995; 57(1):289-300). A false discovery rate (FDR) <0.05 was used as the statistical threshold for DE tests.

Based on an FDR of less than 0.05, 1163 genes were identified as significantly differentially expressed between intermediate-state (e.g., day 18) and earlier-state (e.g., day 13) reference cell populations. These genes are listed in Table E1. Also based on an FDR of less than 0.05, 949 genes were identified as significantly differentially expressed between intermediate-state (e.g., day 18) and later-state (e.g., day 25) reference cell populations. These genes are listed in Table E2.

Other genes differentially expressed between reference cell populations were identified. Cell cycle genes were more enriched in the earlier cultures compared to day 25; these include CCNB2, AURKB, PTTG1 and TOP2A. Also, transcription factors associated with neural precursors (NEUROG2, HES1, HES5, REST) tended to be higher at day 13 and day 18 compared to day 25, while transcription factors associated with specific dopaminergic neurogenesis, such as LMX1A and NR4A2 (NURR1) were enriched in day 25 cultures. Genes associated with developing neural precursors (NES, SOX2, SOX9, RFX4) were more highly expressed at the earlier stages, while genes expressed in dopaminergic neurons were more highly expressed at day 25, including TH, DDC, PBX1, PITX3 and RET. Some genes associated with astrocytes, such as GFAP and SLC1A, were identified at the two later stages, as were markers of oligodendrocytes (OLIG2) and genes associated with vascular leptomeningeal cells (COL1A1 and COL1A2).

Transcription binding site motifs for the transcription factors E2F4, FOXM1, SIN3A and NFYA were enriched in genes expressed at higher levels in day 18 cultures compared to the later stage (day 25). Among the genes up-regulated at the later stage (day 25) relative to day 18, there was a strong enrichment for transcription factor binding site motifs for REST, SUZ12, EZH2 and SMAD4.

REST (RE1 silencing transcription factor) codes for a transcription factor that acts as a repressor of genes involved in neural maturation, and its expression is thought to allow a pool of neural precursors to accumulate during processes of neural differentiation in embryogenesis. REST was detected in the earlier cell stages, but decreased considerably at day 25; in addition, at day 25, genes with REST transcription factor binding motifs were upregulated, which is consistent with removal of REST suppression.

Gene Ontology (GO) analysis indicated that the genes that were up-regulated at day 18 relative to day 25 were largely associated with proliferation. On day 25, the up-regulated genes had an overwhelming signal of synapse-related ontologies.

Some genes that were specifically upregulated in day 18 cells are associated with neurite outgrowth, including LIN28A, FLRT3, and ITGA5. LIN28A (log 2FC=3.8) codes for a post-transcriptional regulator of miRNAs associated with embryogenesis and has been linked to axonal regeneration. Overexpression of LIN28A in DA neurons has been reported to increase dendrite length, graft volume, and TH+ content, and enhance functional recovery post-transplantation. FLRT3, which was upregulated in day 18 cells, is implicated in neurite outgrowth and has been identified as a positive regulator of FGF signaling and cell adhesion. FLRT3 codes for a co-receptor for Robo1; the attractive response to the guidance cue Netrin1 has been shown to be controlled by Slit/Robo1 signaling and by FLRT3. Thus, the expression of FLRT3 may promote neurite outgrowth from the grafted day 18 precursors. ITGA5 codes for subunit alpha 5 in the integrin alpha chain family (Integrin α5β1), which has been identified as having a role in specific dopaminergic neuron outgrowth onto striatal neurons.

TABLE E1
Differentially Expressed Genes (Earlier vs. Intermediate; FDR < 0.05)

| | | | | | |
|---|---|---|---|---|---|
| A2M | ABAT | ABCA17P | ABCA3 | ABCA4 | ABCA5 |
| ABCA8 | ABCD2 | ABCG1 | ABHD15 | ABRAXAS1 | AC002094.5 |
| AC002310.4 | AC002407.1 | AC005538.2 | AC005674.2 | AC006511.5 | AC006547.2 |
| AC007192.1 | AC007731.5 | AC007744.1 | AC008770.4 | AC009084.3 | AC010247.2 |
| AC010616.1 | AC010655.4 | AC010931.2 | AC011472.1 | AC012184.2 | AC012306.3 |
| AC012447.1 | AC012513.3 | AC012651.1 | AC016717.2 | AC018665.1 | AC022336.2 |
| AC022424.1 | AC027228.2 | AC068700.1 | AC068831.4 | AC073323.1 | AC087190.3 |
| AC090114.2 | AC092683.1 | AC092919.2 | AC093010.3 | AC093525.5 | AC093772.1 |
| AC093772.2 | AC093899.2 | AC096589.2 | AC096773.1 | AC104841.1 | AC106886.5 |
| AC107223.1 | AC110285.2 | AC110619.1 | AC120114.4 | AC125232.1 | AC125807.2 |
| AC130304.1 | AC132812.1 | AC132938.5 | AC135178.3 | AC138409.1 | AC138430.1 |
| AC144831.1 | AC159540.2 | AC243919.1 | ACAP3 | ACKR1 | ACRBP |
| ACSS3 | ACTB | ACVR1 | ADAMTS15 | ADAMTS16 | ADAMTS20 |
| ADAMTS9 | ADAMTSL1 | ADAMTSL4 | ADCY2 | ADCYAP1 | ADCYAP1R1 |
| ADGRA1 | ADRA2A | ADTRP | AEN | AF131215.5 | AFAP1 |
| AFF3 | AHCY | AJAP1 | AJUBA | AK4 | AKAP6 |
| AKR1A1 | AL022313.4 | AL032819.2 | AL034430.1 | AL035461.3 | AL049838.1 |
| AL109615.3 | AL109811.1 | AL136169.1 | AL136295.1 | AL138899.2 | AL139142.2 |
| AL157838.1 | AL157871.1 | AL157895.1 | AL158151.1 | AL353147.1 | AL356740.1 |
| AL359643.2 | AL359851.1 | AL359921.2 | AL365203.2 | AL391261.2 | AL513477.1 |
| AL513534.2 | AL590560.3 | AL596223.2 | AL596244.1 | ALCAM | ALDOA |
| ALDOC | ALG11 | ALK | ALMS1-IT1 | ALPL | AMOTL2 |
| ANK3 | ANKRD33B | ANKRD36 | ANKS1B | ANKUB1 | ANO1 |
| ANO4 | ANTXR1 | ANXA1 | AP000295.1 | AP000894.2 | AP000944.5 |
| AP001033.2 | AP001207.3 | AP001350.2 | AP002784.1 | AP002847.1 | AP005329.2 |
| AP3B2 | APBA1 | APC2 | APLN | APOB | APOE |
| ARFGAP3 | ARHGAP24 | ARHGAP29 | ARHGAP39 | ARHGAP45 | ARHGEF19 |
| ARID5B | ARMC3 | ARMH4 | ARRB1 | ARRDC3 | ARRDC4 |
| ARSB | ARSD | ARSE | ASCL1 | ASNS | ASS1 |
| ATIC | ATP10D | ATP1A2 | ATP1A3 | ATP6VOD2 | ATP6V1B1 |
| ATP8A2 | ATXN1 | B4GALNT1 | BCAT1 | BCOR | BHLHE40 |
| BMI1 | BMP6 | BMPR2 | BNIP3 | BRPF3 | BSN |
| BST2 | BTAF1 | BTBD9 | BTF3 | BX284668.2 | BZW2 |
| C11orf95 | C14orf132 | C1orf100 | C1orf115 | C1orf158 | C1orf189 |
| C1orf54 | C1QL1 | C1QL4 | C21orf62 | C2CD4A | C5orf49 |
| C9orf24 | C9orf72 | CA8 | CACNA1B | CACNA1C | CACNA1G |
| CACNA2D1 | CACNG4 | CADM2 | CADM3 | CADPS | CADPS2 |
| CALB1 | CALB2 | CAMK2B | CAMK2D | CAPN5 | CAPN6 |
| CAPN9 | CAPRIN2 | CARMIL3 | CBLN1 | CCDC103 | CCDC184 |
| CCDC190 | CCDC92 | CCKAR | CCN1 | CCN2 | CCN3 |
| CCNB1IP1 | CD36 | CD68 | CDC25B | CDCA7L | CDH10 |
| CDH11 | CDH20 | CDHR3 | CDK6 | CDKN1A | CDKN2B |
| CDO1 | CEBPA-DT | CFAP126 | CFAP43 | CFAP44 | CFAP54 |
| CHCHD10 | CHGA | CHKA | CHPF2 | CHRM2 | CHRNB2 |
| CIC | CICP14 | CICP3 | CLCN2 | CLCNKA | CLDN5 |
| CLEC19A | CLSTN2 | CNR1 | CNTF | CNTFR | CNTNAP1 |
| CNTNAP3C | COL13A1 | COL14A1 | COL22A1 | COL23A1 | COL25A1 |
| COL3A1 | COL4A1 | COL5A2 | COL9A2 | COQ8A | CORIN |
| COX7C | CPE | CPEB2 | CPLX3 | CRABP1 | CRABP2 |
| CRIP2 | CRLF1 | CRYZL2P- | CSGALNACT1 | CSPG5 | CSRNP1 |
| | | SEC16B | | | |
| CTNNA2 | CTXN1 | CU633904.1 | CX3CL1 | CXCL12 | CXCR4 |
| CYP27C1 | CYP2S1 | CYTH2 | DARS | DCAF17 | DCHS1 |
| DCN | DCXR | DDB2 | DDIT4 | DDX3Y | DENND1C |
| DENND5B | DEUP1 | DGKI | DHCR24 | DKK2 | DKK3 |
| DLC1 | DLK1 | DLL3 | DMKN | DMRTA2 | DNAJA1 |
| DNAJC19 | DNAJC22 | DNER | DOCK10 | DOCK6 | DOK3 |
| DPP10 | DPYSL4 | DPYSL5 | DRAM2 | DRD2 | DSCAM |
| DTNA | DTX1 | DTX4 | DUSP15 | DYM | EBF2 |
| EBF3 | EDA2R | EDIL3 | EEF1A1P19 | EEF1B2 | EEF1G |
| EFNB1 | EHBP1 | EHD2 | EIF2S3 | EIF3D | EIF3E |
| EIF3H | EIF3L | EIF3M | EIF4A2 | EIF4B | EIF5 |
| ELFN2 | ELK4 | ELOVL3 | EMD | EME2 | ENC1 |
| ENKUR | ENO1 | ENO2 | ENO3 | ENOX1 | EOMES |
| EPB41L3 | EPB41L4A | EPB41L4A- | EPC2 | EPHA2 | EPHA5 |
| | | AS1 | | | |
| EPPK1 | EPRS | EPS8 | ERBB3 | ERICH3 | ERMP1 |
| ERO1B | ERP27 | ESAM | ESPN | ESRRB | EVI5L |
| EXOC7 | F11R | F13A1 | F3 | FAIM2 | FAM107A |
| FAM122B | FAM135B | FAM13C | FAM155A | FAM155B | FAM162A |
| FAM193B | FAM20C | FAM215B | FAM220A | FAM89B | FAR2P2 |
| FAT3 | FBL | FBN2 | FBRSL1 | FBXL16 | FBXL19-AS1 |
| FBXL7 | FBXO32 | FDXR | FER1L4 | FER1L6 | FGD5-AS1 |
| FGF14 | FGFRL1 | FIBIN | FILIP1L | FKBP4 | FLVCR1 |
| FMN1 | FNDC1 | FNDC9 | FOS | FOXA3 | FOXP2 |
| FRAS1 | FRMD3 | FSIP2 | FST | FTH1P16 | FUT3 |
| FXYD1 | FZD1 | GABRA1 | GABRA3 | GABRB3 | GABRQ |
| GADD45A | GAP43 | GAPDH | GAS5 | GCNT1 | GDF1 |
| GDF11 | GDF15 | GDPD2 | GJA3 | GK5 | GLI3 |
| GLRA2 | GNA11 | GNG11 | GOLGA8CP | GPC1 | GPCPD1 |
| GPD1L | GPI | GPR1 | GPR161 | GPR35 | GPR62 |
| GPR85 | GPRASP1 | GPRIN1 | GRAMD2A | GRB14 | GRHL2 |
| GRIN2B | GRIN3A | GRK5 | GRM3 | GSN | GUCY1A2 |
| GUK1 | H1F0 | H1FX-AS1 | HAPLN1 | HAUS4 | HCN4 |
| HEATR5A | HECTD2 | HECW1 | HEPACAM2 | HES1 | HES6 |
| HES7 | HIPK2 | HIST2H2AA3 | HIVEP3 | HK2 | HKDC1 |
| HLA-E | HMGA1 | HNRNPA1P61 | HNRNPM | HPRT1 | HPS4 |
| HS3ST5 | HSD17B8 | HSF1 | HSPA4L | HSPG2 | ICA1 |
| ICAM5 | ID1 | ID2 | ID3 | IER5 | IGDCC3 |
| IGF1R | IGSF10 | IL10RB-DT | IL13RA2 | IL1RAPL1 | IL22RA1 |
| IL5RA | IMPDH1 | IMPDH2 | IMPG2 | INA | INMT- |
| | | | | | MINDY4 |
| INPP5D | INTS6P1 | IPO5 | IPO5P1 | IQCE | IQCN |
| IRS1 | ISLR2 | ITGB8 | ITIH3 | JAG1 | JPH4 |
| KALRN | KAT2A | KCND3 | KCNF1 | KCNH1 | KCNIP4 |
| KCNJ13 | KCNJ16 | KCNJ2 | KCNMA1 | KCNMB2 | KCTD12 |
| KCTD8 | KDR | KIF5A | KIFC2 | KLF10 | KLHDC8A |
| KLHL1 | KLHL21 | KLHL41 | KMO | KPNA2P3 | KRTAP5-AS1 |
| KYAT3 | LANCL2 | LBR | LDHA | LDHAP4 | LDLRAD4 |
| LEFTY2 | LEPROT | LETMD1 | LFNG | LGALS3BP | LGI1 |
| LGMN | LIF | LIMCH1 | LIN28A | LINC00173 | LINC00261 |
| LINC00342 | LINC00461 | LINC00488 | LINC00641 | LINC00643 | LINC00680 |
| LINC00689 | LINC00858 | LINC00930 | LINC01014 | LINC01305 | LINC01445 |
| LINC01522 | LINC01963 | LINC02523 | LINC02525 | LINC02730 | LINC02751 |
| LITAF | LIX1 | LMCD1 | LPAR5 | LPGAT1 | LRCH1 |
| LRP10 | LRP1B | LRPPRC | LRRC24 | LRRC4 | LRRC55 |
| LRRN3 | LTA4H | LUM | LUZP2 | LY6G5C | LY6H |
| LZTS1 | MAGI1 | MAGI2 | MALRD1 | MAMDC2 | MANEAL |
| MAP1LC3B2 | MAP2 | MAP2K6 | MAP3K19 | MAP3K6 | MAPK8IP1 |
| MAPK8IP2 | MAPKAPK5- | MAPT | MATN2 | MBTPS1 | MDGA1 |
| | AS1 | | | | |
| MEGF10 | MEGF8 | MEGF9 | MGST1 | MIF | MIR124-2HG |
| MIR1915HG | MIR217HG | MIR29B2CHG | MIR34AHG | MIR99AHG | MKRN3 |
| MLLT1 | MLLT6 | MMP24 | MMRN1 | MOV10 | MRC2 |
| MRPL10 | MRPL9P1 | MRTFB | MSRB3 | MSX1 | MTFP1 |
| MTG1 | MT-RNR1 | MTUS2 | MUC1 | MXD4 | MYL9 |
| MYLIP | MYO5A | NAA25 | NACA | NACAD | NAMPT |
| NCAM1 | NCOA5 | NDST1 | NECAB1 | NEFL | NEFM |
| NEGR1 | NETO2 | NEURL1B | NFIB | NGF | NGFR |
| NHLH2 | NHSL1 | NMD3 | NMNAT2 | NOB1 | NOP53 |
| NOVA1 | NOX3 | NPAS3 | NPC2 | NPTXR | NPY |
| NR4A2 | NR6A1 | NRG1 | NRP2 | NRSN1 | NSG1 |
| NTN1 | NTRK3 | NWD2 | NXF2 | NXF2B | OCA2 |
| OCSTAMP | OLA1 | OLFM3 | ONECUT3 | OSBPL10 | OSTC |
| OTOL1 | P4HA1 | P4HA2 | PABPC1 | PABPC1L2B | PACS2 |
| PAICS | PAK3 | PAPSS2 | PCDH1 | PCDH7 | PCDHA4 |
| PCDHGB6 | PCNT | PCOLCE-AS1 | PCYT1B | PCYT2 | PDE3A |
| PDE4B | PDE4D | PDE4DIP | PDE5A | PDK1 | PDZK1P1 |
| PDZRN4 | PELI3 | PFKFB4 | PGK1 | PGM1 | PHLDA3 |
| PHTF1 | PHYH | PHYHIPL | PIDD1 | PIEZO2 | PIK3R3 |
| PKM | PLCD1 | PLCD4 | PLCL2 | PLEKHA6 | PLEKHG2 |
| PLEKHO1 | PLIN4 | PLK2 | PLK3 | PLOD2 | PLPP4 |
| PLPPR1 | PLPPR3 | PLXDC2 | PLXNA2 | PMEL | PMEPA1 |
| PMP22 | PNCK | PNMA1 | PNRC1 | POLR2H | POLR3H |
| POSTN | POU2F2 | POU3F1 | PPA1 | PPAT | PPFIA4 |
| PPFIBP2 | PPID | PPP1R14BP3 | PPP1R3B | PPP2R2B | PRDX4 |
| PRICKLE2 | PRKAB2 | PROM1 | PRR15 | PRRT1B | PSMA3-AS1 |
| PSMD10P2 | PSPC1 | PTENP1 | PTPN3 | PTPRO | PTPRZ1 |
| PXMP2 | QPRT | RAD52 | RALGPS1 | RAMP1 | RASGRF2 |
| RASL12 | RAX | RCAN1 | RCAN2 | RDH5 | RGPD2 |
| RGS11 | RGS4 | RGS5 | RHOBTB2 | RHOQ | RIMBP3B |
| RIMKLA | RIMS1 | RIPK1 | RLF | RMST | RN7SKP23 |
| RND3 | RNF128 | RPL10 | RPL10A | RPL11 | RPL12 |
| RPL13 | RPL13A | RPL14 | RPL15 | RPL17 | RPL18 |
| RPL18A | RPL19 | RPL21 | RPL21P16 | RPL22 | RPL22P1 |
| RPL23 | RPL23A | RPL23AP42 | RPL24 | RPL26 | RPL27 |
| RPL27A | RPL28 | RPL29 | RPL3 | RPL30 | RPL31 |
| RPL32 | RPL32P29 | RPL34 | RPL35 | RPL35A | RPL36 |
| RPL36A | RPL36AL | RPL37 | RPL37A | RPL38 | RPL39 |
| RPL4 | RPL5 | RPL6 | RPL7 | RPL7A | RPL7AP10 |
| RPL8 | RPL9 | RPLP0 | RPLP1 | RPLP2 | RPS10 |
| RPS11 | RPS12 | RPS13 | RPS14 | RPS15 | RPS15A |
| RPS16 | RPS17 | RPS18 | RPS19 | RPS2 | RPS20 |
| RPS21 | RPS23 | RPS24 | RPS25 | RPS27 | RPS27A |
| RPS27L | RPS28 | RPS29 | RPS2P5 | RPS3 | RPS3A |
| RPS4X | RPS4Y1 | RPS5 | RPS6 | RPS7 | RPS8 |
| RPS9 | RPSA | RSL24D1 | RSPH4A | RTL9 | RTN1 |
| RTN2 | RUNX2 | RXRG | SACS-AS1 | SAMD11 | SAMD3 |
| SAMD5 | SARM1 | SCG2 | SCGB2B2 | SCML1 | SCN3A |
| SCN3B | SCN7A | SCN9A | SCUBE1 | SEC11A | SERPINF1 |
| SERPINI2 | SESN1 | SFPQ | SFT2D3 | SFXN3 | SHANK2 |
| SHC2 | SHC3 | SHISA7 | SHISA9 | SHLD2P3 | SHMT2 |
| SHROOM3 | SIK2 | SIL1 | SKAP1 | SLC12A2 | SLC13A4 |
| SLC16A3 | SLC17A6 | SLC17A7 | SLC17A8 | SLC18A1 | SLC1A2 |
| SLC20A2 | SLC22A18AS | SLC22A23 | SLC23A2 | SLC25A40 | SLC25A53 |
| SLC26A6 | SLC27A2 | SLC27A3 | SLC29A1 | SLC2A1 | SLC2A3 |
| SLC30A2 | SLC32A1 | SLC35E1P1 | SLC37A1 | SLC38A1 | SLC38A10 |
| SLC38A2 | SLC44A2 | SLC4A3 | SLC6A8 | SLIT3 | SLITRK4 |
| SMOC2 | SMPDL3A | SNAI1 | SNAP25 | SNHG1 | SNHG16 |
| SNHG29 | SNHG32 | SNHG5 | SNHG6 | SNHG8 | SNTB1 |
| SNX29 | SOGA1 | SORBS2 | SORL1 | SORT1 | SOX21-AS1 |
| SOX2-OT | SOX9 | SP5 | SPAG4 | SPAG6 | SPATA7 |
| SPDYE6 | SPECC1 | SPINT1 | SPTBN5 | SRARP | SRRM4 |
| SRSF3 | SRSF6 | SSX2IP | ST18 | ST8SIA3 | ST8SIA4 |
| STAC | STAG1 | STARD9 | STIMATE | STK24 | STK32A |
| STMN3 | STOM | STS | SUCLG2 | SULF1 | SUMF2 |
| SV2B | SYBU | SYNGAP1- | SYNM | SYNPO2 | SYNPR |
| | | AS1 | | | |
| SYT10 | SYT14 | SYT4 | SYT5 | TAC1 | TACR2 |
| TAF1D | TAGLN | TANC2 | TATDN3 | TBC1D9 | TCTA |
| TCTEX1D1 | TENT5C | TESC | TEX15 | TEX261 | TFDP1 |
| TFDP2 | TFF3 | TFPI2 | TGM2 | THAP9-AS1 | THBS1 |
| THBS4 | TIMP2 | TKT | TM4SF18 | TM7SF2 | TMCO3 |
| TMEFF2 | TMEM101 | TMEM159 | TMEM163 | TMEM178B | TMEM200A |
| TMEM256 | TMEM63C | TMEM64 | TMEM68 | TMOD1 | TMOD2 |
| TMPRSS13 | TMPRSS3 | TNC | TNFRSF10B | TNFRSF10D | TNFRSF12A |
| TNFSF15 | TNIK | TOMM20 | TOP1MT | TOX2 | TP53INP2 |
| TP63 | TPD52L1 | TPH1 | TPI1 | TRABD2A | TRAM2 |
| TRAP1 | TRAPPC11 | TRIM36 | TRIM67 | TRIM69 | TRIM71 |
| TRIQK | TRPM4 | TRPM8 | TSPAN7 | TTYH1 | TUB |
| TXLNB | TXNIP | UBA52 | UBD | UBE2QL1 | UBTF |
| UCHL1 | UFL1 | UGT3A1 | UNC119 | UNC5B | UQCRB |
| VASH2 | VAV3 | VCAN | VEGFA | VEGFD | VGLL3 |
| VLDLR | VTN | VWA5B1 | VWA5B2 | VWC2 | WDR45 |
| WDR54 | WRB- | XKR4 | XPO4 | YBX3 | ZACN |
| | SH3BGR | | | | |
| ZBTB20 | ZBTB40 | ZC3H4 | ZFAS1 | ZFHX4-AS1 | ZFP36L1 |
| ZFY | ZFYVE28 | ZMAT3 | ZMPSTE24 | ZNF138 | ZNF271P |
| ZNF280C | ZNF330 | ZNF362 | ZNF397 | ZNF451 | ZNF474 |
| ZNF48 | ZNF605 | ZNF700 | ZNF804A | ZNF860 | |

TABLE E2
Differentially Expressed Genes (Later vs. Intermediate; FDR < 0.05)

| | | | | | |
|---|---|---|---|---|---|
| AASS | ABCA1 | ABCA13 | ABLIM3 | AC002310.4 | AC005786.3 |
| AC006027.1 | AC006547.2 | AC007098.1 | AC007192.1 | AC007614.1 | AC007938.3 |
| AC008581.2 | AC009084.3 | AC009133.4 | AC010247.2 | AC010422.6 | AC010463.1 |
| AC010616.1 | AC010729.1 | AC011446.3 | AC011511.4 | AC016717.2 | AC019197.1 |
| AC022966.1 | AC023055.1 | AC026401.3 | AC027031.2 | AC080100.1 | AC091078.1 |
| AC093458.2 | AC098582.1 | AC099521.2 | AC104083.1 | AC104461.1 | AC124068.1 |
| AC138430.1 | ACAA2 | ACBD7 | ACHE | ACSS3 | ACTL6B |
| ACTN1 | ACVR2A | ADAM28 | ADAMTS1 | ADAMTS12 | ADAMTS16 |
| ADAMTS7 | ADAMTSL2 | ADARB2 | ADCY8 | ADCYAP1 | ADD2 |
| ADGRG2 | ADGRG6 | ADGRL1 | ADSS | AEBP1 | AGAP2 |
| AJAP1 | AJUBA | AK4 | AL021395.1 | AL096711.2 | AL133325.3 |
| AL136295.1 | AL139142.2 | AL161665.1 | AL162417.1 | AL162586.2 | AL356123.2 |
| AL357093.2 | ALDH1A1 | ALDH3B2 | ALK | AMBN | AMER3 |
| AMPD3 | AMPH | ANK1 | ANK2 | ANK3 | ANKRD33B |
| ANLN | ANP32E | ANXA11 | AP000894.2 | AP005329.2 | AP1M2 |
| AP3B2 | APC2 | APELA | APOA1 | APOB | APOE |
| ARG2 | ARHGAP11A | ARHGAP19 | ARHGAP23 | ARHGAP8 | ARHGDIG |
| ARHGEF17 | ARL4A | ARMCX7P | ARRDC3 | ARSI | ASNS |
| ASPH | ASPHD1 | ASPM | ATCAY | ATP1A3 | ATP2A3 |
| ATP6V1G2 | ATP8A1 | ATP8A2 | AURKA | AURKB | AUXG01000058.1 |
| B4GALT6 | BACH2 | BARD1 | BCAR3 | BCAT1 | BCL11A |
| BICDL1 | BIRC5 | BLACAT1 | BLOC1S5- | BMPER | BOC |
| | | | TXNDC5 | | |
| BORA | BPTFP1 | BRCA1 | BRINP3 | BRSK1 | BRSK2 |
| BSCL2 | BSN | BSPRY | BUB1 | BUB1B | C18orf54 |
| C1orf116 | C1orf198 | C1orf21 | C1QL1 | C22orf42 | C4orf50 |
| C6orf141 | C8orf88 | CA11 | CA14 | CACNA1B | CACNA2D1 |
| CACNB1 | CACNG7 | CACNG8 | CADM3 | CALCA | CALD1 |
| CAMK1D | CAMK2B | CAMK2N2 | CAP2 | CAPN6 | CARMIL3 |
| CARTPT | CBLN1 | CBLN2 | CBX6 | CCDC150 | CCDC184 |
| CCDC80 | CCN1 | CCN2 | CCNA2 | CCNB1 | CCNB2 |
| CD248 | CD302 | CD83 | CD99 | CDC20 | CDC25C |
| CDCA2 | CDCA7L | CDCA8 | CDH1 | CDH3 | CDHR2 |
| CDK1 | CDK5R1 | CDK6 | CDKN1A | CDO1 | CELF3 |
| CELF4 | CELF5 | CELF6 | CELSR3 | CENPE | CENPF |
| CENPH | CENPI | CENPU | CEP135 | CEP55 | CEP63 |
| CGA | CHGA | CHGB | CHRNB2 | CHRNB3 | CHST14 |
| CICP3 | CIP2A | CIT | CKAP2 | CKAP2L | CKS1B |
| CKS2 | CLASP2 | CLCN4 | CLDN4 | CLDN7 | CLVS1 |
| CLVS2 | CMIP | CMTM3 | CNGB1 | CNN2 | CNR1 |
| CNTFR | CNTN1 | CNTNAP5 | COL18A1 | COL22A1 | COL25A1 |
| COL27A1 | COL2A1 | COL4A1 | COL4A2 | COL5A1 | COL5A2 |
| COL9A1 | COLEC12 | CPEB3 | CPNE8 | CPS1 | CPXM1 |
| CRB2 | CRB3 | CRISPLD1 | CRTAP | CRYBA1 | CSRNP3 |
| CTIF | CTNNA2 | CTNND2 | CTSC | CTSZ | CU634019.1 |
| CYB561 | CYB5R2 | CYP1B1 | CYP27A1 | CYYR1 | DAAM2 |
| DAB2 | DBF4 | DBNDD1 | DCLK1 | DCX | DDIAS |
| DDIT4 | DECR1 | DENNDIC | DEPDC1 | DEPDC1B | DGKI |
| DISP2 | DLGAP3 | DLGAP5 | DLK1 | DMTN | DNAJC6 |
| DNER | DNM3 | DNMBP | DOCK1 | DRD2 | DSCAM |
| DTX1 | DUSP16 | DUSP4 | DUSP8 | EBF1 | ECT2 |
| EEF1A2 | EFEMP1 | EFEMP2 | EGLN3 | ELAVL2 | ELAVL3 |
| ELMO1 | ENO2 | EPB41L2 | EPCAM | EPHA5 | EPHA6 |
| EPHB1 | EPHB4 | EPN3 | EPS8L2 | ERBB3 | ERCC6L |
| ESPL1 | ESRP1 | ESRP2 | EVPL | FABP7 | FAM13A |
| FAM155A | FAM163A | FAM171B | FAM219A | FAM72D | FAM83D |
| FANCD2 | FANCI | FAXC | FBLN1 | FBN1 | FBN2 |
| FBXL16 | FEZ1 | FIGNL2 | FLRT3 | FLVCR2 | FMNL1 |
| FNDC1 | FOXJ1 | FOXM1 | FSTL1 | FSTL4 | FSTL5 |
| FUOM | FXYD7 | FZD2 | GABRG2 | GALNT10 | GALNT6 |
| GALNT9 | GALNTL6 | GAP43 | GAREM1 | GAS2L3 | GASK1B |
| GATA2 | GCNT1 | GDAP1 | GDAP1L1 | GGH | GINS1 |
| GJA1 | GJB2 | GLRA2 | GLYATL3 | GNA14 | GNAO1 |
| GNB3 | GNG3 | GNG4 | GOLGA6L22 | GP1BB | GPR137C |
| GPRIN1 | GPX8 | GRB14 | GRIA1 | GRIA2 | GRID1 |
| GRIK2 | GRIK5 | GRIN3A | GRIP2 | GRM3 | GRM7 |
| GRM8 | GTSE1 | GUCY1A1 | GULP1 | H2AFX | HAUS6 |
| HCN3 | HCN4 | HECW1 | HECW2 | HEG1 | HELLS |
| HEPH | HES 1 | HIST2H2AA4 | HJURP | HLA-DOA | HLA-E |
| HMGB2 | HMMR | HOOK1 | HPCAL4 | HRK | HS3ST1 |
| HS6ST3 | HSPG2 | HTATIP2 | HTR1A | IFITM3 | IGF2BP3 |
| IGSF9B | IL18 | ILDR2 | INA | INAVA | INCENP |
| INHBA | IQGAP2 | IQGAP3 | IQSEC3 | IRF6 | IRF8 |
| ITGA5 | ITGAV | ITGB5 | ITGB6 | JAM2 | JPH4 |
| JUN | KALRN | KCNA1 | KCNA6 | KCNB1 | KCNC1 |
| KCNC2 | KCND3 | KCNF1 | KCNH1 | KCNH4 | KCNH6 |
| KCNJ9 | KCNMB1 | KCNMB2 | KCNQ2 | KDF1 | KIAA0408 |
| KIAA0930 | KIF11 | KIF12 | KIF14 | KIF18A | KIF19 |
| KIF1A | KIF20A | KIF20B | KIF21B | KIF23 | KIF2C |
| KIF3C | KIF4A | KIF5A | KIF5C | KIFC1 | KIFC2 |
| KIRREL1 | KLF7 | KLHL1 | KNL1 | KNTC1 | KPNA2 |
| KPNA2P3 | KRT7 | KRT77 | KRTAP5-1 | KRTAP5-2 | KRTAP5-AS1 |
| KSR2 | L1CAM | LAMA1 | LAMB1 | LAMC1 | LANCL3 |
| LARGE2 | LBH | LGALS1 | LGALS3BP | LGR5 | LHFPL2 |
| LHFPL4 | LIN28A | LIN28B | LINC00622 | LINC00645 | LINGO1 |
| LMTK3 | LONRF2 | LPIN3 | LRATD2 | LRP1B | LRRC3B |
| LRRC4C | LRRC55 | LRTM1 | LUZP1 | MAD2L1 | MAL2 |
| MAMDC2 | MAN2A1 | MANEAL | MAP3K20 | MAP3K9 | MAP7D3 |
| MAPK13 | MAPK8IP1 | MAPK8IP2 | MAPRE3 | MAPT | MARCH4 |
| MAST1 | MATN3 | MCF2L | MDFI | MDFIC | MDGA1 |
| MDK | MEF2C | MELK | METTL7A | MFAP2 | MGAT4C |
| MGST1 | MIAT | MICAL2 | MIR503HG | MIR7-3HG | MIS18BP1 |
| MISP | MKI67 | MLLT11 | MLPH | MMP14 | MMP17 |
| MMP2 | MMP24 | MMRN1 | MPP5 | MRC2 | MSC-AS1 |
| MSL3P1 | MSN | MSRB3 | MSX1 | MTMR7 | MTSS1 |
| MTURN | MTUS2 | MUC5AC | MUC5B | MUSTN1 | MYL12A |
| MYL4 | MYL9 | MYLK | MYO10 | MYOF | MYRF |
| MYT1 | MYTIL | NAALAD2 | NAPB | NAV1 | NAV2 |
| NBPF10 | NBPF19 | NCAM1 | NCAN | NCAPD2 | NCAPG |
| NCAPG2 | NDC80 | NDE1 | NDRG4 | NEFL | NEK2 |
| NEK6 | NEMP1 | NEUROD1 | NEXMIF | NEXN | NFASC |
| NFIB | NFIX | NID1 | NKAIN2 | NKD1 | NLRX1 |
| NMNAT2 | NOL4 | NOS1AP | NPC1L1 | NPTX1 | NPTXR |
| NR6A1 | NRXN1 | NRXN2 | NSG1 | NSG2 | NUDT4B |
| NUF2 | NUP210 | NUSAP1 | NXF2 | NYAP1 | OCSTAMP |
| OLIG2 | ONECUT2 | ONECUT3 | OPLAH | OSBPL10 | OSBPL3 |
| OTX1 | OVOL2 | PAK5 | PARP6 | PARPBP | PBK |
| PCDH1 | PCDH10 | PCDH17 | PCDH18 | PCDHA1 | PCOLCE |
| PDE1A | PDE5A | PDIA2 | PDLIM2 | PDYN | PDZD4 |
| PGA4 | PGA5 | PGM2L1 | PHACTR1 | PHF19 | PHF21B |
| PHTF1 | PHYHIPL | PIF1 | PIK3C2G | PIMREG | PKDCC |
| PKIA | PLA2G4F | PLCE1 | PLCH1 | PLEKHA6 | PLIN2 |
| PLIN3 | PLK1 | PLK4 | PLOD1 | PLP2 | PLPP3 |
| PLPPR2 | PLTP | PLXNA2 | PLXNA4 | PMEL | PNRC2 |
| PODXL | POSTN | POU2F2 | POU4F1 | PPFIA3 | PPFIBP2 |
| PPP1R13B | PPP1R17 | PPP2R2B | PPP2R5A | PPP2R5B | PRC1 |
| PREX2 | PRKAR2B | PRKCB | PRPS1 | PRR11 | PRR15L |
| PRRT4 | PRRX1 | PRSS23 | PRSS8 | PRTG | PSD |
| PSD2 | PSRC1 | PTPN13 | PTPN14 | PTPRE | PTPRT |
| PTTG1 | PXYLP1 | RAB25 | RAB30 | RAB34 | RAB3A |
| RAB3C | RAB6B | RABGAP1L | RACGAP1 | RAI14 | RALGPS2 |
| RAPGEF6 | RASL11B | RASSF5 | RASSF6 | RBBP8NL | RBM24 |
| RBM47 | REEP1 | RET | RGS17 | RGS5 | RGS7 |
| RGS7BP | RGS8 | RIMS3 | RIMS4 | RNF112 | RNF152 |
| RNF43 | ROR1 | RPRM | RPS6KL1 | RTKN2 | RTL1 |
| RTN1 | RUNDC3A | RUNX1T1 | RUSC2 | RYR1 | SALL1 |
| SALL4 | SAMD11 | SCAMP5 | SCG3 | SCN2A | SCN3A |
| SCN3B | SCN9A | SCNN1A | SCRT1 | SDCBP | SELENBP1 |
| SELENOP | SEMA5B | SEPTIN10 | SEPTIN5 | SERPING1 | SERPINH1 |
| SERTAD4 | SEZ6L | SFRP1 | SFRP2 | SFXN3 | SGIP1 |
| SGO1 | SGO2 | SH2D3C | SH3BP5 | SH3GL3 | SH3TC1 |
| SHANK1 | SHB | SHISA7 | SHROOM3 | SIM1 | SIX2 |
| SKA1 | SLC12A5 | SLC12A8 | SLC16A1 | SLC1A2 | SLC27A2 |
| SLC35D2 | SLC35F1 | SLC39A8 | SLC4A10 | SLC4A8 | SLC7A2 |
| SLC7A5 | SLC8A2 | SLC8A3 | SLCO5A1 | SMAD3 | SMAP2 |
| SMC4 | SMIM18 | SMOC2 | SMPD3 | SMPDL3A | SNAP25 |
| SNAP91 | SNPH | SOBP | SORBS1 | SORCS1 | SORCS3 |
| SOX1-OT | SP6 | SPAG5 | SPARC | SPHKAP | SPINT1 |
| SPINT2 | SPRED3 | SRCIN1 | SRRM3 | SRRM4 | SS18L1 |
| ST14 | ST8SIA3 | ST8SIA6 | STIL | STK3 | STMN2 |
| STMN3 | STMN4 | STOM | STON1 | STOX1 | STOX2 |
| STX1A | STX1B | STXBP1 | SULT4A1 | SV2A | SV2B |
| SVOP | SYN1 | SYP | SYT1 | SYT13 | SYT16 |
| SYT4 | SYT5 | SYT7 | TAC1 | TACC3 | TAGLN |
| TBC1D3I | TCAF2 | TCF7L2 | TEAD2 | TEK | TENM1 |
| TENT5B | TERF2IP | TFRC | TGFB2 | TGIF1 | TGIF2 |
| TGM2 | TH | THBS1 | THBS4 | THSD1 | TIMELESS |
| TLCD3B | TLE2 | TMC4 | TMEM121B | TMEM130 | TMEM163 |
| TMEM176A | TMEM178B | TMEM189- | TMEM196 | TMEM63C | TMOD2 |
| | | UBE2V1 | | | |
| TMPRSS4 | TNC | TNFRSF19 | TOB1 | TOP2A | TOX2 |
| TPH1 | TPM2 | TPX2 | TRABD2B | TRIM59 | TRIM67 |
| TRIP6 | TROAP | TSPAN12 | TSPAN18 | TSPAN7 | TTBK1 |
| TTC9B | TTK | TTYH2 | TUBA1C | TUBB6 | TWSG1 |
| TXLNB | UACA | UBE2C | UBE2QL1 | UCHL1 | UNC13A |
| UNC79 | USP2 | UTRN | VCL | VEGFD | VGF |
| VIM | VRK1 | VSTM2A | VTN | VXN | WDFY2 |
| WDR47 | WEE2 | WNT4 | WNT5A | WWC2 | WWTR1 |
| XKR4 | XKR7 | YAP1 | Z83844.1 | ZCCHC12 | ZFP36L2 |
| ZHX3 | ZNF107 | ZNF217 | ZNF385D | ZNF540 | ZNF804A |
| ZWINT | | | | | |

### F. Model Training

A machine learning method as described in Example 1 for identifying cell populations having an intermediate differentiation state is developed using expression levels of the genes identified as significantly differentially expressed (e.g., genes listed in Table E1 and Table E2). The machine learning method is trained and developed using gene expression levels from reference cell populations at earlier, intermediate, and later differentiation states.

Novelty score training and cutoff development is performed as described in Example 1. Training of a first machine learning model (Model 1) is performed as described in Example 1 using gene expression levels from earlier-state and intermediate-state reference cell populations. Expression levels of the genes listed in Table E1 are used for Model-1 training. Training of a second machine learning model (Model 2) is performed as described in Example 1 using gene expression levels from later-state and intermediate-state reference cell populations. Expression levels of the genes listed in Table E2 are used for Model-2 training. Model output cutoffs are determined and validated using test cell populations as described in Example 1.

### Example 3: Machine Learning Method for Identifying Hematopoietic Progenitors Cells

The machine learning method described in Example 1 was used for training multiple machine learning models to discriminate between microglial cell populations of different differentiation states. Training data included RNAseq data collected from iPSCs (earlier state; n=4), hematopoietic progenitor cells (iHPCs; intermediate state; n=3), and iPSC-derived microglial cells (iMGLs; later state; n=6) during their culture in an exemplary differentiation protocol involving the in vitro culture of iPSCs under conditions to differentiate the cells to microglial cells (see Abud et al. (2017) Neuroresource 94(2):P278-293.E9). The first PCA machine learning model was trained to discriminate between test cell populations having expression levels similar to iPSC populations (earlier state) or to iHPC populations (intermediate state). The second PCA machine learning model was trained to discriminate between test cell populations having expression levels similar to iHPC populations (intermediate state) or iMGL populations (later state).

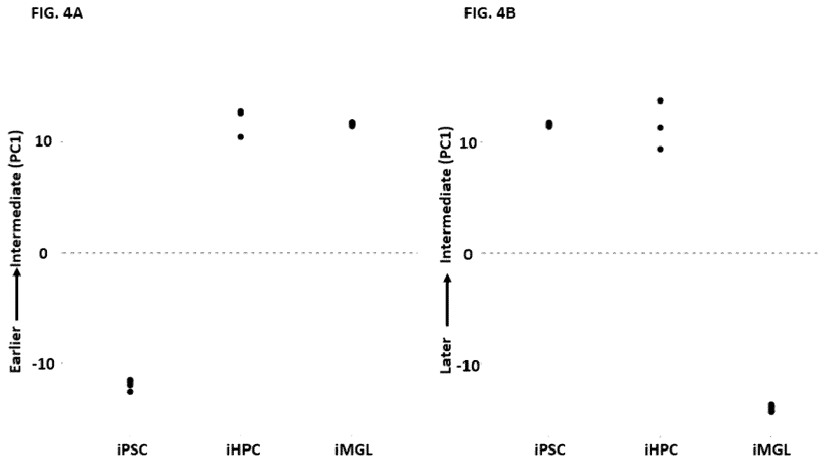

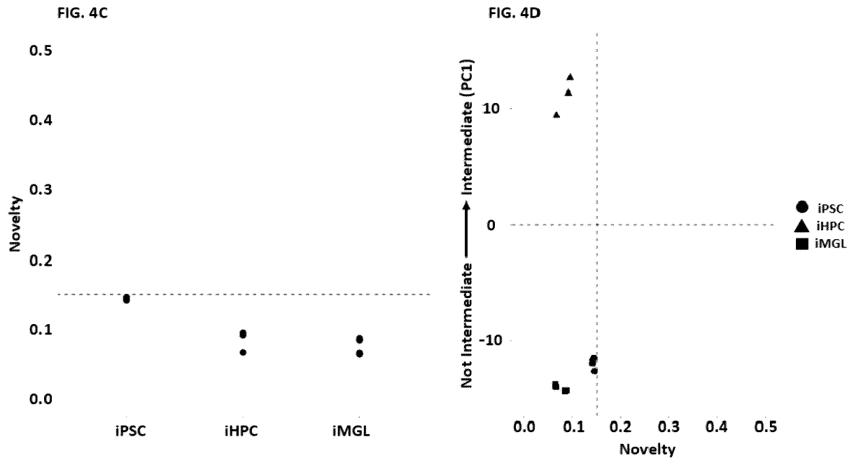

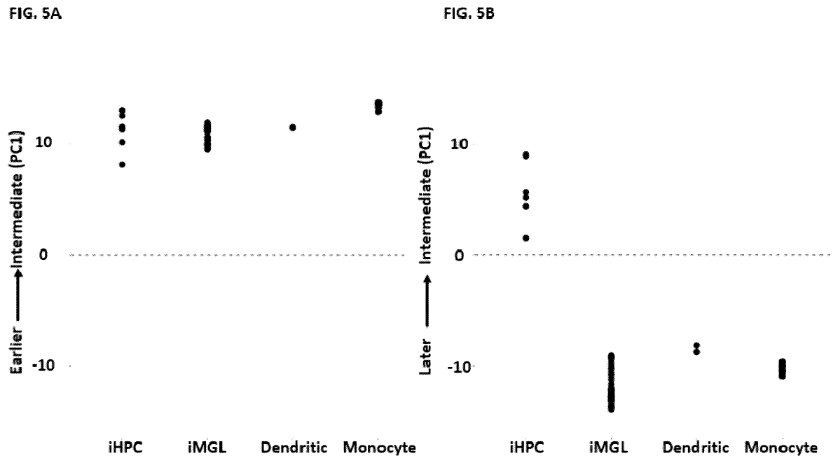

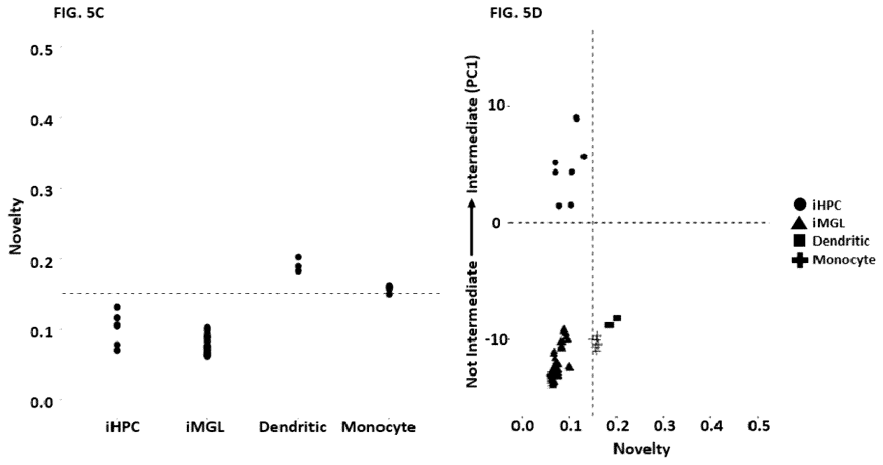

Results for reference cell populations used for model training are shown in FIG. 4A-4D. Model validation results with test cell populations not used for model training are shown in FIG. 5A-5D.

FIG. 4A shows the results of the first PCA model trained to discriminate between iPSC populations (earlier state) and iHPC populations (intermediate state). As shown in FIG. 4A, iHPC populations (intermediate state) and iMGL populations (later state) had PC1 values distinguishable from iPSC populations (earlier state). FIG. 4B shows the results of the second PCA model trained to discriminate between iHPC populations (intermediate state) and iMGL populations (later state). As shown in FIG. 4B, iPSC populations (earlier state) and iHPC populations (intermediate state) had PC1 values distinguishable from iMGL populations (later state). FIG. 4C shows the novelty scores calculated for the reference cell populations, all of which were below the novelty score cutoff of 0.15. FIG. 4D shows the results of both PCA models together, with y-axis values reflecting the minimum PC1 value between models. As shown in FIG. 4D, iHPC populations (intermediate state; triangles) had minimum PC1 values distinguishable from iPSC populations (earlier state; circles) and iMGL populations (later state; squares).

RNAseq data collected from a separate set of test cell populations not used for model training was used for model validation. The test cell populations were generated using an alternative differentiation protocol that was not used to produce the reference cell populations used for model training (see McQuade et al. (2018) Molecular Neurodegeneration 13:67). The test cell populations included iHPC populations (n=9) and iMGL populations (n=51). To test for the identification of cell populations having an alternative differentiation fate from that of microglial cells, the test cell populations also included dendritic cell populations (n=3), and monocyte populations (n=9).

FIG. 5A shows the validation results for the first PCA model trained to discriminate between iPSC populations (earlier state) and iHPC populations (intermediate state). As shown in FIG. 5A, all test cell populations had comparable PC1 values. FIG. 5B shows the validation results for the second PCA model trained to discriminate between iHPC populations (intermediate state) and iMGL populations (later state). As shown in FIG. 5B, iHPC populations (intermediate state) had PC1 values distinguishable from all other test cell populations. FIG. 5C shows the novelty scores calculated for the test cell populations. iHPC populations (intermediate state) and iMGL populations (later state) all had novelty scores below the novelty score cutoff of 0.15, whereas all dendritic cell populations and most monocyte populations had novelty scores greater than 0.15. FIG. 5D shows the results of both PCA models together, with y-axis values reflecting the minimum PC1 value between models. As shown in FIG. 5D, iHPC populations (intermediate state; circles) had minimum PC1 values distinguishable from iMGL populations (later state; triangles), dendritic cell populations (squares), and monocyte populations (plus marks).

Overall, these results show that the developed machine learning model was able to accurately identify cell populations at an intermediate microglial differentiation state (iHPCs), including for test cell populations produced using alternative differentiation protocols. The machine learning method was also able to identify cell populations having an alternative differentiation fate from that of microglial cells.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

1. A computing device for classifying the differentiation state of an in vitro population of cells, the device comprising a memory that comprises:
a first reference dataset that comprises a representation of gene expression levels for one or more genes that are differentially expressed between cells at a first differentiation state and cells at a second differentiation state; and
a second reference dataset that comprises a representation of gene expression levels for one or more genes that are differentially expressed between cells at the second differentiation state and cells at a third differentiation state.

2. The computing device of claim 1, further comprising a processor that implements instructions stored in the memory to perform a method comprising:
(a) receiving as input a test dataset that comprises expression levels for genes that are expressed in one or more test cells comprised in an in vitro population of cells, wherein the expression levels in the test dataset comprise expression levels for (i) one or more of the genes for which a representation of expression levels are included in the first reference dataset, and (ii) one or more of the genes for which a representation of expression levels are included in the second reference dataset;
(b) calculating, using the test dataset and the first reference dataset, a first similarity score indicating whether the differentiation state of the test cells is more similar to the first differentiation state or to the second differentiation state;
(c) calculating, using the test dataset and the second reference dataset, a second similarity score indicating whether the differentiation state of the test cells is more similar to the second differentiation state or to the third differentiation state; and
(d) classifying the differentiation state of the one or more test cells based on one or both of the first similarity score and the second similarity score.

3. The computing device of claim 1, wherein the memory further comprises a control dataset that comprises a representation of gene expression levels for one or more genes that are expressed in cells at one or more control differentiation states, which control differentiation state may be the same as or different than one of the first, second, or third differentiation states.

4. The computing device of claim 2, wherein:
the memory further comprises a control dataset that comprises a representation of gene expression levels for one or more genes that are expressed in cells at one or more control differentiation states, which control differentiation state may be the same as or different than one of the first, second, or third differentiation states;
the test dataset comprises gene expression levels for one or more of the genes for which a representation of expression levels are included in the control dataset;
the instructions comprise calculating a degree of correlation between the representation of gene expression levels for one or more genes in the control dataset and gene expression levels for the one or more genes in the test dataset to calculate a correlation score; and
the classifying the differentiation state of the one or more test cells is based on the correlation score and the one or both of the first similarity score and the second similarity score.

5-9. (canceled)

10. The computing device of claim 2, wherein the population of cells is from a culture of cells differentiated from pluripotent cells that are subjected to suitable differentiation conditions.

11. The computing device of claim 1, wherein the first differentiation state is earlier in a stem cell differentiation pathway than the second differentiation state and/or the second differentiation state is earlier in a stem cell differentiation pathway than the third differentiation state.

12-13. (canceled)

14. The computing device of claim 2, wherein the population of cells are selected from the group consisting of stem-cell derived cardiac muscle cells, stem-cell derived skeletal muscle cells, stem-cell derived kidney tubule cells, stem-cell derived red blood cell cells, stem-cell derived smooth muscle cells, stem-cell derived lung cells, stem-cell derived thyroid cells, stem-cell derived pancreatic cells, stem-cell derived epidermal cells, stem-cell derived pigment cells, and stem-cell derived neuronal cells.

15. (canceled)

16. The computing device of claim 1, wherein the second differentiation state is the differentiation state of a determined dopaminergic neuronal cell.

17. The computing device of claim 1, wherein the second differentiation state is the differentiation state of a hematopoietic progenitor cell.

18. The computing device of claim 1, wherein the first reference dataset comprises a representation of gene expression levels for one or more genes selected from Table E1 and/or the second reference dataset comprises a representation of gene expression levels for one or more genes selected from Table E2.

19. (canceled)

20. The computing device of claim 1, wherein the first reference dataset comprises a representation of gene expression levels for at least 20 genes selected from Table E1 and/or the second reference dataset comprises a representation of gene expression levels for at least 20 genes selected from Table E2.

21-29. (canceled)

30. The computing device of claim 1, wherein the representations of gene expression levels in the first reference dataset is a machine learning model trained on gene expression levels for the one or more genes differentially expressed between cells at the first differentiation state and cells at the second differentiation state and/or the representation of gene expression levels in the second reference dataset is a machine learning model trained on gene expression levels for the one or more genes differentially expressed between cells at the second differentiation state and cells at the third differentiation state.

31-33. (canceled)

34. A method for selecting a population of cells having a desired differentiation state, the method comprising:
(a) calculating a first similarity score using a test dataset and a first reference dataset, wherein:
the first reference dataset comprises a representation of gene expression levels for one or more genes that are differentially expressed between cells at a first differentiation state and cells at a second differentiation state,
the test dataset comprises expression levels for genes that are expressed in one or more test cells comprised in an in vitro population of cells, wherein the expression levels in the test dataset comprise expression levels for one or more of the genes for which a representation of expression levels are included in the first reference dataset, and
the first similarity score indicates whether the differentiation state of the test cells is more similar to the first differentiation state or to the second differentiation state;
(b) calculating a second similarity score using the test dataset and a second reference dataset, wherein:
the second reference dataset comprises a representation of gene expression levels for one or more genes that are differentially expressed between cells at the second differentiation state and cells at a third differentiation state,
the expression levels in the test dataset comprise expression levels for one or more of the genes for which a representation of expression levels are included in the second reference dataset, and
the second similarity score indicates whether the differentiation state of the test cells is more similar to the second differentiation state or to the third differentiation state; and
(c) classifying the differentiation state of the one or more test cells based on one or both of the first similarity score and the second similarity score.

35. The method of claim 34, wherein:
the test dataset comprises gene expression levels for one or more genes for which a representation of expression levels are included in a control dataset that comprises a representation of gene expression levels for one or more genes that are expressed in cells at a control differentiation state, which control differentiation state may be the same as or different than one of the first, second, or third differentiation states;
the method further comprises calculating a degree of correlation between the representation of gene expression levels for one or more genes in the control dataset and gene expression levels for the one or more genes in the test dataset to calculate a correlation score; and
the classifying the differentiation state of the one or more test cells is based on the correlation score and the one or both of the first similarity score and the second similarity score.

36-40. (canceled)

41. The method of claim 34, wherein the first differentiation state is earlier in a stem cell differentiation pathway than the second differentiation state and/or the second differentiation state is earlier in a stem cell differentiation pathway than the third differentiation state.

42-43. (canceled)

44. The method of claim 34, wherein the population of cells are selected from the group consisting of stem-cell derived cardiac muscle cells, stem-cell derived skeletal muscle cells, stem-cell derived kidney tubule cells, stem-cell derived red blood cell cells, stem-cell derived smooth muscle cells, stem-cell derived lung cells, stem-cell derived thyroid cells, stem-cell derived pancreatic cells, stem-cell derived epidermal cells, stem-cell derived pigment cells, and stem-cell derived neuronal cells.

45. (canceled)

46. The method of claim 34, wherein the second differentiation state is the differentiation state of a determined dopaminergic neuronal cell.

47. The method of claim 34, wherein the second differentiation state is the differentiation state of a hematopoietic progenitor cell.

48. The method of claim 34, wherein the first reference dataset comprises a representation of gene expression levels for one or more genes selected from Table E1 and/or the second reference dataset comprises a representation of gene expression levels for one or more genes selected from Table E2.

49. (canceled)

50. The method of claim 34, wherein the first reference dataset comprises a representation of gene expression levels for at least 20 genes selected from Table E1 and/or the second reference dataset comprises a representation of gene expression levels for at least 20 genes selected from Table E2.

51-59. (canceled)

60. The method of claim 34, wherein the representations of gene expression levels in the first reference dataset is a machine learning model trained on gene expression levels for the one or more genes differentially expressed between cells at the first differentiation state and cells at the second differentiation state and/or the representation of gene expression levels in the second reference dataset is a machine learning model trained on gene expression levels for the one or more genes differentially expressed between cells at the second differentiation state and cells at the third differentiation state.

61-63. (canceled)

64. The method of claim 34, wherein the one or more test cells are classified as having the second differentiation state, and the method further comprises selecting the in vitro population of cells comprising the one or more test cells as having the desired differentiation state.

65. A method for implanting a population of cells having a desired differentiation state into a subject, the method comprising:
(a) selecting a population of cells having a desired differentiation state using the method of claim 34; and
(b) implanting the population of cells into a subject.

66-67. (canceled)

68. A pharmaceutical composition comprising a pharmaceutical carrier and a population of cells having a desired differentiation state, wherein the cells are selected using the method of claim 34.

69-71. (canceled)

72. A method for training a machine learning model to classify the differentiation state of an in vitro population of cells, the method comprising:
(a) obtaining, for a plurality of reference populations of cells, gene expression levels for one or more genes that are differentially expressed between cells at a first differentiation state and cells at a second differentiation state and applying the gene expression levels as input to train a first machine learning model to predict if an in vitro population of cells comprises one or more test cells having a differentiation state that is more similar to the first differentiation state or to the second differentiation state; and
(b) obtaining, for a plurality of reference populations of cells, gene expression levels for one or more genes that are differentially expressed between cells at the second differentiation state and cells at a third differentiation state and applying the gene expression levels as input to train a second machine learning model to predict if an in vitro population of cells comprises one or more test cells having a differentiation state that is more similar to the second differentiation state or to the third differentiation state.

73. A method for training a machine learning model to classify the differentiation state of an in vitro population of cells, the method comprising:
(a) selecting one or more genes that are differentially expressed between cells at a first differentiation state and cells at a second differentiation state and applying expression levels of the selected genes for a plurality of reference populations of cells as input to train a first machine learning model to predict if an in vitro population of cells comprises one or more test cells having a differentiation state that is more similar to the first differentiation state or to the second differentiation state; and
(c) selecting one or more genes that are differentially expressed between cells at the second differentiation state and cells at a third differentiation state and applying expression levels of the selected genes for a plurality of reference populations of cells as input to train a second machine learning model to predict if an in vitro population of cells comprises one or more test cells having a differentiation state that is more similar to the second differentiation state or to the third differentiation state.

74. (canceled)

75. A method for selecting a population of cells having a desired differentiation state, comprising:
(a) obtaining a test dataset comprising gene expression levels of one or more genes selected from AC010247.2, ANKRD33B, APC2, AQP4, ASCL1, AURKB, BARHL2, CACNA1G, CAPN6, CBLN1, CCNB2, CDH1, CDH20, CHGA, COL1A1, COL1A2, COL22A1, COL4A1, CRABP1, DBX1, DCN, DCX, DDC, DOCK10, E2F4, EDNRB, ESRP1, EZH2, FABP7, FBLN1, FLRT3, FOXA2, FOXM1, GAP43, GFAP, GFRA1, GJA1, GLRA2, HES1, HES2, HES5, ITGA5, JPH4, LDHA, LIN28A, LIX1, LMX1A, LUM, NCAM1, NES, NEUROG2, NGFR, NKX2-2, NMNAT2, NPTX1, NR4A2, NR4A2 (NURR1), NSG2, NFYA, OLFM3, OLIG1, OLIG2, OTX2, P4HA1, PBX1, PDGFRA, PIEZO2, PITX3, PLP1, PMEL, PMP2, POSTN, POU2F2, PPP2R2B, PRTG, PTTG1, REST, RET, RFX4, RFX4, SALL4, SIN3A, SLC16A3, SLC18A2, SLC1A, SLC1A2, SLC1A3, SLC4A4, SMAD4, SNAP25, SOX10, SOX2, SOX9, STMN2, SUZ12, SV2B, SYN1, SYT1, SYT13, TH, TOP2A, TPH1, TPM2, and TXNIP for one or more test cells comprised in an in vitro population of cells; and
(b) applying the gene expression levels as input to a process configured to predict if the population of cells has a desired differentiation state.

76. The method of claim 75, wherein the in vitro population of cells comprises stem-cell derived neuronal cells.

77. The method of claim 75, wherein the desired differentiation state is the differentiation state of a determined dopaminergic neuronal cell.

78. A method for selecting a population of cells predicted to exhibit neurite outgrowth following implantation in a brain region, comprising:
(a) obtaining a test dataset comprising gene expression levels of one or more genes selected from AC010247.2, ANKRD33B, APC2, AQP4, ASCL1, AURKB, BARHL2, CACNA1G, CAPN6, CBLN1, CCNB2, CDH1, CDH20, CHGA, COL1A1, COL1A2, COL22A1, COL4A1, CRABP1, DBX1, DCN, DCX, DDC, DOCK10, E2F4, EDNRB, ESRP1, EZH2, FABP7, FBLN1, FLRT3, FOXA2, FOXM1, GAP43, GFAP, GFRA1, GJA1, GLRA2, HES1, HES2, HES5, ITGA5, JPH4, LDHA, LIN28A, LIX1, LMX1A, LUM, NCAM1, NES, NEUROG2, NGFR, NKX2-2, NMNAT2, NPTX1, NR4A2, NR4A2 (NURR1), NSG2, NFYA, OLFM3, OLIG1, OLIG2, OTX2, P4HA1, PBX1, PDGFRA, PIEZO2, PITX3, PLP1, PMEL, PMP2, POSTN, POU2F2, PPP2R2B, PRTG, PTTG1, REST, RET, RFX4, RFX4, SALL4, SIN3A, SLC16A3, SLC18A2, SLC1A, SLC1A2, SLC1A3, SLC4A4, SMAD4, SNAP25, SOX10, SOX2, SOX9, STMN2, SUZ12, SV2B, SYN1, SYT1, SYT13, TH, TOP2A, TPH1, TPM2, and TXNIP for one or more test cells comprised in an in vitro population of cells; and
(b) applying the gene expression levels as input to a process configured to predict if the population of cells will exhibit neurite outgrowth following implantation in a brain region.

79. (canceled)

80. The method of claim 75, wherein the process comprises a machine learning model trained using gene expression levels of the one or more genes, and the method further comprises classifying the differentiation state of the one or more test cells based on one or more outputs of the machine learning model.

81. (canceled)

82. The method of claim 78, wherein the process comprises a machine learning model trained using gene expression levels of the one or more genes, and the method further comprises predicting if the test cells will exhibit neurite outgrowth following implantation in a brain region based on one or more outputs of the machine learning model.

83. A pharmaceutical composition comprising a pharmaceutical carrier and a population of neuronal cells, wherein the cells are selected using the method of claim 75.

84. An in vitro stem cell-derived neuronal cell population comprising cells that express one or more genes selected from the group consisting of CCNB2, AURKB, PTTG1, TOP2A, NEUROG2, HES1, REST, E2F4, FOXM1, SIN3A, NFYA, LIN28A, FLRT3, ITGA5, NES, SOX2, SOX9, and RFX4.

85. The in vitro stem-cell derived neuronal cell population of claim 84, wherein:
(1) at least one gene from the one or more genes is selected from the group consisting of CCNB2, AURKB, PTTG1, TOP2A, NEUROG2, HES1, REST, E2F4, FOXM1, SIN3A, NFYA, LIN28A, FLRT3, and ITGA5; and
(2) at least one gene from the one or more genes is selected from the group consisting of NES, SOX2, SOX9, and RFX4.

86-114. (canceled)

115. A pharmaceutical composition comprising a pharmaceutical carrier and the in vitro stem-cell derived neuronal cell population of claim 84.

116-121. (canceled)

122. A method of treatment, comprising implanting in a brain region of a subject in need thereof a therapeutically effective amount of the pharmaceutical composition of claim 115.

123-132. (canceled)

133. A pharmaceutical composition comprising a pharmaceutical carrier and a population of neuronal cells, wherein the cells are selected using the method of claim 78.

134. A method of treatment, comprising implanting in a brain region of a subject in need thereof a therapeutically effective amount of the pharmaceutical composition of claim 133.
